(19) United States
(12) Patent Application Publication
(10) Pub. No.: US 2004/0009477 A1
(43) Pub. Date: Jan. 15, 2004

(54) Methods for producing libraries of expressible gene sequences

(75) Inventors: Joseph M. Fernandez, Carlsbad, CA (US); John A. Heyman, Rixensart (BE); James P. Hoeffler, Anchorage, AK (US); Heather L. Marks-Hull, Oceanside, CA (US); Michelle L. Sindici, San Diego, CA (US)

Correspondence Address: LISA A. HAILE, Ph.D., GRAY CARY WARE & FREIDENRICH LLP, Suite 1100, 4365 Executive Drive, San Diego, CA, 92121-2133, US

(73) Assignee: INVITROGEN CORPORATION

(21) Appl. No.: 09/990,091

(22) Filed: Nov. 21, 2001

Related U.S. Application Data
(63) Continuation of application No. 09/843,281, filed on Apr. 25, 2001, now abandoned.
Application No. 09/843,281 is a continuation of application No. 09/647,651, now abandoned.
Application No. 09/647,651 is a 371 of international application No. PCT/US99/07270, filed on Apr. 2, 1999.
Application No. PCT/US99/07270 is a continuation-in-part of application No. 09/054,936, filed on Apr. 3, 1998, now abandoned.

(30) Foreign Application Priority Data
Apr. 2, 1999 (US) US99/07270

(51) Int. Cl. C12Q 1/68; C12P 19/34

(52) U.S. Cl. 435/6; 435/91.2

(57) ABSTRACT

The present invention comprises a method for producing libraries of expressible gene sequences. The method of the invention allows for the simultaneous manipulation of multiple gene sequences and thus allows libraries to be created in an efficient and high throughput manner. The expression vectors containing verified gene sequences can be used to transfect cells for the production of recombinant proteins. The invention further comprises libraries of expressible gene sequences produced using the method of the invention and expression vectors used in the construction of said libraries.

### FIELD OF THE INVENTION

The invention disclosed herein relates to the fields of genomics and molecular biology. More specifically the invention relates to new high through-put methods of making libraries of expressed gene sequences and the libraries made using said methods.

### BACKGROUND OF THE INVENTION

Recent breakthroughs in nucleic acid sequencing technology have made possible the sequencing of entire genomes from a variety of organisms, including humans. The potential benefits of a complete genome sequence are many, ranging from applications in medicine to a greater understanding of evolutionary processes. These benefits cannot be fully realized, however, without an understanding of how and where these newly sequenced genes function.

Traditionally, functional understanding started with recognizing an activity, isolating a protein associated with that activity, then identifying and isolating the gene, or genes, encoding that protein. Each gene of interest was identified, isolated and expressed separately, a relatively time consuming process.

Recently, breakthroughs in high through-put DNA sequencing technology have allowed massive amounts of gene sequence information to become available to the public. Yet methods of expressing these sequences to produce the proteins encoded by them for study have still required that each sequence be manipulated one at a time. Accordingly, a need exists for the development of methods for the rapid, simultaneous expression of large numbers of gene sequences. The invention described herein addresses this and related needs as will become apparent upon inspection of the specification and the appended claims.

### BRIEF DESCRIPTION OF THE INVENTION

The present invention comprises a method for producing libraries of expressible gene sequences. The method of the invention allows for the simultaneous manipulation of multiple gene sequences and thus allows libraries to be created in an efficient and high through-put manner. The expression vectors containing verified gene sequences can be used to transfect cells for the production of recombinant proteins. The invention method utilizes known techniques in such a way as to create an efficient high through-put means of producing libraries of expressible gene sequences.

The invention further comprises libraries of expressible gene sequences produced using the method of the invention and expression vectors used in the construction of such libraries.

### BRIEF DESCRIPTION OF THE FIGURE

FIG. 1 shows a schematic representation of the vaccinia topoisomerase type I cloning method used in the practice of the invention.

### BRIEF DESCRIPTION OF THE INVENTION

The present invention comprises a method for producing libraries of expressible gene sequences. The invention method comprises the following steps: amplifying a plurality of gene sequences, purifying the amplified gene sequences, inserting each of the purified gene sequences into an expression vector, and verifying the size and orientation of the inserted gene sequence.

In the first step, the gene sequences that are to be expressed are amplified. By “amplification” it is meant that the copy number of the gene sequence(s) is increased. One commonly used method of amplification is the polymerase chain reaction (PCR). In brief, starter DNA is heat-denatured into single strands. Two synthetic oligonucleotides, one complementary to sequence at the 3′ end of the sense strand of DNA segment of interest and the other complementary to the sequence at the 3′ end of the anti-sense strand of a DNA segment of interest, are added in great excess to the DNA sequence to be amplified and the temperature is lowered to 50-60° C. The specific oligonucleotides hybridize with the complementary sequences in the DNA and then serve as primers of DNA chain synthesis, which requires the addition of a supply of deoxynucleotides and a temperature-resistant DNA polymerase, such as Taq polymerase, which can extend the primers at temperatures up to 72° C. When synthesis is complete, the whole mixture is heated further (up to 95° C.) to melt the newly formed DNA duplexes. When the temperature is lowered again, another round of synthesis takes place, since an excess of primer is still present. Repeated cycles of synthesis and melting quickly amplify the sequence of interest. A more detailed description of PCR can be found in Erlich, Ed, PCR Technology: Principles and Applications for DNA Amplification, W. H. Freeman and Co., 1992 and Erlich, et al, Eds., Polymerase Chain Reaction, Cold Spring Harbor Laboratory, 1989, both of which are incorporated by reference herein.

Starter DNA can come from a variety of sources. It can be total genomic DNA from an organism, for example, or can be cDNA that has been synthesized from cellular mRNA using reverse transcriptase. Sources of suitable RNA include normal and diseased tissues, cellular extracts, and the like.

In practicing the method of the invention, the desired gene sequences can come from any source. The examples presented below show the amplification of all open reading frames (ORFs) from a single organism, Saccharomyces cerevisiae, for example. By “open reading frame” it is meant a segment of DNA that exists between a start codon and a stop codon and is likely to represent a gene. The examples presented below further show the amplification of a group of human genes thought to be important in the development of cancer.

Public databases exist that contain the entire or partial genome of a particular organism, for example yeast (Saccharomyces cerevisiae), prokaryotes (Bacillus subtilis, E. coli, Borrelia burgdorferi, Helicobacter pylori, Mycoplasma genitalium, and the like), fish (Fugu rubripes), mammals (human, mouse), plants (rice, cotton) and the like. Well known databases include GenBank, Unigene, EMBL, IMAGE and TIGR, for example. Public databases such as these can be used a source of gene sequences for use in the method of the invention.

The primers employed in the amplification step are specific for each desired gene sequence and include a variety of unique features. For example, the 5′ “sense” primer starts with the sequence 5′-CACCATG . . . (the start codon is underlined). The CACC sequence is added as a Kozak consensus that aids in translational efficiency. When the gene sequence being amplified represents a full-length gene, the 3′ “antisense” codon is preferably designed to make the amplification product end at the 3rd position of the last codon of the gene being amplified, plus a single adenine residue. This facilitates the fusion of the coding region in-frame with a heterologous peptide sequence such as an epitope tag, an affinity purification tag, and the like (see below). The gene sequence need not encode a full-length sequence, however, as the invention methods are equally suitable for any gene sequence, including Expressed Sequence Tags (ESTs). The primers can be synthesized and dried in multiwell formats, such as 96-well microtiter plates to facilitate identification and further processing.

The amplified gene products are next isolated from the other components of the amplification reaction mixture. This purification can be accomplished using a variety of methodologies such as column chromatography, gel electrophoresis, and the like. A preferred method of purification utilizes low-melt agarose gel electrophoresis. The reaction mixture is separated and visualized by suitable means, e.g. by ethidiun bromide staining. DNA bands that represent correctly sized amplification products are cut away from the rest of the gel and placed into appropriate corresponding wells of a 96-well microtiter plate. These plugs are subsequently melted and the DNA contained therein utilized as cloning inserts. The use of gel electrophoresis has the advantage that the practitioner can purify the desired amplified gene sequence while additionally verifying that the sequence is of the correct size, i.e., represents the entire desired gene sequence.

The purified, amplified gene sequences are next inserted into an expression vector. A variety of expression vectors are suitable for use in the method of the invention, both for prokaryotic expression and eukaryotic expression. In general, the expression vector will have one or more of the following features: a promoter-enhancer sequence, a selection marker sequence, an origin of replication, an affinity purification tag sequence, an inducible element sequence, an epitope-tag sequence, and the like.

Promoter-enhancer sequences are DNA sequences to which RNA polymerase binds and initiates transcription. The promoter determines the polarity of the transcript by specifying which strand will be transcribed. Bacterial promoters consist of consensus sequences, −35 and −10 nucleotides relative to the transcriptional start, which are bound by a specific sigma factor and RNA polymerase. Eukaryotic promoters are more complex. Most promoters utilized in expression vectors are transcribed by RNA polymerase II. General transcription factors (GTFs) first bind specific sequences near the start and then recruit the binding of RNA polymerase II. In addition to these minimal promoter elements, small sequence elements are recognized specifically by modular DNA-binding/trans-activating proteins (e.g. AP-1, SP-1) which regulate the activity of a given promoter. Viral promoters serve the same function as bacterial or eukaryotic promoters and either provide a specific RNA polymerase in trans (bacteriophage T7) or recruit cellular factors and RNA polymerase (SV40, RSV, CMV). Viral promoters are preferred as they are generally particularly strong promoters.

Promoters may be, furthermore, either constitutive or, more preferably, regulatable (i.e., inducible or derepressible). Inducible elements are DNA sequence elements which act in conjunction with promoters and bind either repressors (e.g. lacO/LAC Iq repressor system in E. coli) or inducers (e.g. gal1/GAL4 inducer system in yeast). In either case, transcription is virtually “shut off” until the promoter is derepressed or induced, at which point transcription is “turned-on”.

Examples of constitutive promoters include the int promoter of bacteriophage λ, the bla promoter of the β-lactamase gene sequence of pBR322, the CAT promoter of the chloramphenicol acetyl transferase gene sequence of pPR325, and the like. Examples of inducible prokaryotic promoters include the major right and left promoters of bacteriophage (P_{L} and P_{R}), the trp, reca, lacZ, LacI, AraC and gal promoters of E. coli, the α-amylase (Ulmanen et al., J. Bacteriol. 162:176-182, 1985) and the sigma-28-specific promoters of B. subtilis (Gilman et al., Gene sequence 32:11-20(1984)), the promoters of the bacteriophages of Bacillus (Gryczan, In: The Molecular Biology of the Bacilli, Academic Press, Inc., NY (1982)), Streptomyces promoters (Ward et al., Mol. Gen. Genet. 203:468-478, 1986), and the like. Exemplary prokaryotic promoters are reviewed by Glick (J. Ind. Microbiol. 1:277-282,1987); Cenatiempo (Biochimie 68:505-516,1986); and Gottesman (Ann. Rev. Genet 18:415-442, 1984).

Preferred eukaryotic promoters include, for example, the promoter of the mouse metallothionein I gene sequence (Hamer et al., J. Mol. Appl. Gen. 1:273-288, 1982); the TK promoter of Herpes virus (McKnight, Cell 31:355-365, 1982); the SV40 early promoter (Benoist et al., Nature (London) 290:304-310, 1981); the yeast gal1 gene sequence promoter (Johnston et al., Proc. Natl. Acad. Sci. (USA) 79:6971-6975, 1982); Silver et al., Proc. Natl. Acad. Sci. (USA) 81:5951-5955, 1984), the CMV promoter, the EF-1 promoter, Ecdysone-responsive promoter(s), and the like.

Selection marker sequences are valuable elements in expression vectors as they provide a means to select for growth only those cells which contain a vector. Such markers are of two types: drug resistance and auxotrophic. A drug resistance marker enables cells to detoxify an exogenously added drug that would otherwise kill the cell. Auxotrophic markers allow cells to synthesize an essential component (usually an amino acid) while grown in media which lacks that essential component.

Common selectable marker gene sequences include those for resistance to antibiotics such as ampicillin, tetracycline, kanamycin, streptomycin, bleomycin, hygromycin, neomycin, Zeocin™, and the like. Selectable auxotrophic gene sequences include, for example, hisD, which allows growth in histidine free media in the presence of histidinol.

A preferred selectable marker sequence for use in yeast expression systems is URA3. Laboratory yeast strains carrying mutations in the gene which encodes orotidine-5′-phosphate decarboxylase, an enzyme essential for uracil biosynthesis, are unable to grow in the absence of exogenous uracil. A copy of the wild-type gene (ura4+in S. pombe and URA3 in S. cerevisiae) will complement this defect in trans.

A further element useful in an expression vector is an origin of replication sequence. Replication origins are unique DNA segments that contain multiple short repeated sequences that are recognized by multimeric origin-binding proteins and which play a key role in assembling DNA replication enzymes at the origin site. Suitable origins of replication for use in expression vectors employed herein include E. coli oriC, 2μ and ARS (both useful in yeast systems), sf1, SV40 (useful in mammalian systems), and the like.

Additional elements that can be included in expression vectors employed in the invention method are sequences encoding affinity purification tags or epitope tags. Affinity purification tags are generally peptide sequences that can interact with a binding partner immobilized on a solid support. Synthetic DNA sequences encoding multiple consecutive single amino acids, such as histidine, when fused to the expressed protein, may be used for one-step purification of the recombinant protein by high affinity binding to a resin column, such as nickel sepharose. An endopeptidase recognition sequence is often engineered between the polyamino acid tag and the protein of interest to allow subsequent removal of the leader peptide by digestion with a specific protease. Sequences encoding peptides such as the chitin binding domain (which binds to chitin), glutathione-S-transferase (which binds to glutathione), biotin (which binds to avidin or strepavidin), and the like can also be used for facilitating purification of the protein of interest. The affinity purification tag can be separated from the protein of interest by methods well known in the art, including the use of inteins (protein self-splicing elements, Chong, et al, Gene 192:271-281, 1997).

Epitope tags are short peptide sequences that are recognized by epitope specific antibodies. A fusion protein comprising a recombinant protein and an epitope tag can be simply and easily purified using an antibody bound to a chromatography resin. The presence of the epitope tag furthermore allows the recombinant protein to be detected in subsequent assays, such as Western blots, without having to produce an antibody specific for the recombinant protein itself. Examples of commonly used epitope tags include V5, glutathione-S-transferase (GST), hemaglutinin (HA), the peptide Phe-His-His-Thr-Thr, chitin binding domain, and the like.

A further useful element in an expression vector is a multiple cloning site or polylinker. Synthetic DNA encoding a series of restriction endonuclease recognition sites is inserted into a plasmid vector downstream of the promoter element. These sites are engineered for convenient cloning of DNA into the vector at a specific position.

The foregoing elements can be combined to produce expression vectors useful in the practice of the present invention. Suitable prokaryotic vectors include plasmids such as those capable of replication in E. coli (for example, pBR322, Co1E1, pSC101, PACYC 184, itVX, pRSET, pBAD (Invitrogen, Carlsbad, Calif.) and the like). Such plasmids are disclosed by Sambrook (cf. “Molecular Cloning: A Laboratory Manual”, second edition, edited by Sambrook, Fritsch, & Maniatis, Cold Spring Harbor Laboratory, (1989)). Bacillus plasmids include pC194, pC221, pT127, and the like, and are disclosed by Gryczan (In: The Molecular Biology of the Bacilli, Academic Press, NY (1982), pp. 307-329). Suitable Streptomyces plasmids include plJlOl(Kendall et al, J. Bacteriol. 169:4177-4183,1987), and streptomyces bacteriophages such as φC31 (Chater et al., In. Sixth International Symposium on Actinomycetales Biology, Akademiai Kaido, Budapest, Hungary (1986), pp. 45-54). Pseudomonas plasmids are reviewed by John et al. (Rev. Infect. Dis. 8:693-704, 1986), and Izaki (Jpn. J. Bacteriol. 3:729-742, 1978).

Suitable eukaryotic plasmids include, for example, BPV, vaccinia, SV40, 2-microns circle, pcDNA3.1, pCDNA3. 1/GS, pYES2/GS, pMT, p IND, pIND(Sp1), pVgRXR (Invitrogen), and the like, or their derivatives. Such plasmids are well known in the art (Botstein et al., Miami Wntr. Symp. 19:265-274, 1982); Broach, In: “The Molecular Biology of the Yeast Saccharomyces: Life Cycle and Inheritance”, Cold Spring Harbor Laboratory, Cold Spring Harbor, NY, p. 445-470, 1981; Broach, Cell 28:203-204, 1982; Dilon et al., J. Clin. Hematol. Oncol. 10:39-48, 1980; Maniatis, In: Cell Biology: A Comprehensive Treatise, Vol. 3, Gene Sequence Expression, Academic Press, NY, pp. 563-608, 1980).

Construction of chimaeric DNA molecules in vitro relies traditionally on two enzymatic steps catalyzed by separate protein components. PCR amplification or site-specific restriction endonucleases are used to generate linear DNAs with defined termnini that can then be joined covalently at their ends via the action of DNA ligase. DNA ligase has limitations, however, in that it is relatively slow acting and temperature sensitive.

Thus, when inserting the purified, amplified gene sequence into the expression vector the use of an enzyme that can both cleave and religate DNA in a site specific manner is preferred. Any site-specific enzyme of this type is suitable, for example, a type I topoisomerase or a site-specific recombinase. Examples of suitable site-specific recombinases include lambda integrase, FLP recombinase, P1-Cre protein, Kw recombinase, and the like (Pan, et al, J. Biol. Chem. 268:3683-3689, 1993; Nunes-Duby, et al, EMBO J. 13:4421-4430, 1994; Hallet and Sherratt, FEMS Microbio. Revs 21:157-178, 1997; Ringrose, et al, Eur J. Biochem 248:903-912, 1997).

A particularly suitable enzyme for use in the invention method is a type I topoisomerase, particularly vaccinia DNA topoisomerase. Vaccinia DNA topoisomerase binds to duplex DNA and cleaves the phosphodiester backbone of one strand. The enzyme exhibits a high level of sequence specificity, akin to that of a restriction endonuclease. Cleavage occurs at a consensus pentapyrimidine element 5′-(C/T)CCTT in the scissile strand. In the cleavage reaction, bond energy is conserved via the formation of a covalent adduct between the 3′ phosphate of the incised strand and a tyrosyl residue of the protein. Vaccinia topoisomerase can religate the covalently held strand across the same bond originally cleaved (as occurs during DNA relaxation) or it can religate to a heterologous acceptor DNA and thereby create a recombinant molecule.

When the substrate is configured such that the scissile bond is situated near (within 10 basepairs of) the 3′ end of a DNA duplex, cleavage is accompanied by the spontaneous dissociation of the downstream portion of the cleaved strand. The resulting topoisomerase-DNA complex, containing a 5′ single-stranded tail, can religate to an acceptor DNA if the acceptor molecule has a 5′ OH tail complementary to that of the activated donor complex.

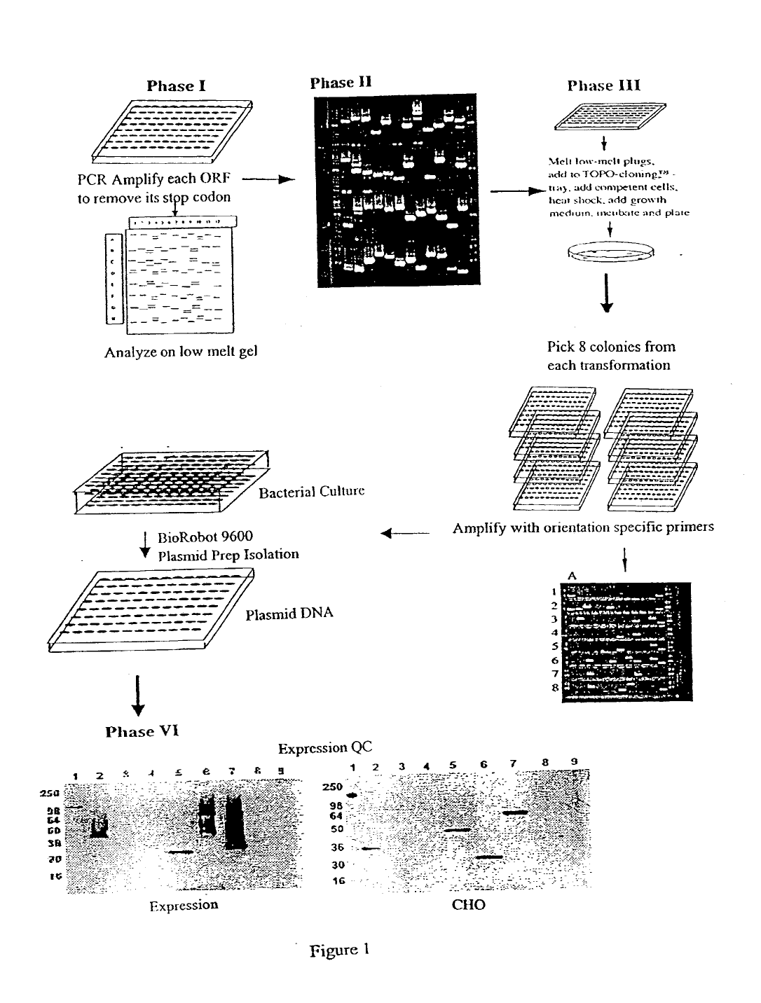

In accordance with the present invention, this reaction has been optimized for joining PCR-amplified DNA fragments into plasmid vectors (See FIG. 1). PCR fragments are naturally good surrogate substrates for the topoisomerase I religation step because they generally have 5′ hydroxyl residues from the primers used for the amplification reaction. The 5′ hydroxyl is the substrate for the religation reactions. The use of vaccinia topoisomerase type I for cloning is described in detail in copending U.S. patent application Ser. No. 08/358,344, filed Dec. 19, 1994, incorporated by reference herein in its entirety.

The gene sequence being inserted into the expression vector can insert in either the sense or antisense direction. Therefore, the invention method provides for verification of both the size and orientation of the insert to insure that the gene sequence will express the desired protein. Preferably, the insert plus vector is utilized in a standard bacterial transformation reaction and the contents of the transformation plated onto selective growth media. Bacterial transformation and growth selection procedures are well known in the art and described in detail in, for example, Ausubel, et al, Short Protocols in Molecular Biology, 3rd ed. 1995.

Individual bacterial colonies are picked and grown in individual wells of a multiwell microtiter plate containing selective growth media. An aliquot of these cells is used directly in a diagnostic PCR reaction. Primers for this reaction are designed such that only plasmids with correctly oriented inserts give amplification product. The amplified DNA is separated and visualized by SDS-PAGE gel electrophoresis using standard protocols (see Ausubel, et al, Short Protocols in Molecular Biology, 3rd ed. 1995).

Performing the PCR reaction directly from the cultured cell lysates, rather than first preparing DNA from the bacteria, is a particular advantage of the invention method as it significantly reduces both the time needed to generate the required data and the cost of doing so.

Once plasmids containing the gene sequence insert in the correct orientation have been identified, plasmid DNA is prepared for use in the transformation of host cells for expression. Methods of preparing plasmid DNA and transformation of cells are well known to those skilled in the art. Such methods are described, for example, in Ausubel, et al, supra.

Prokaryotic hosts are, generally very efficient and convenient for the production of recombinant proteins and are, therefore, one type of preferred expression system. Prokaryotes most frequently are represented by various strains of E. coli. However, other organisms may also be used, including other bacterial strains.

Recognized prokaryotic hosts include bacteria such as E. coli and those from genera such as Bacillus, Streptomyces, Pseudomonas, Salmonella, Serratia, and the like. However, under such conditions, the polypeptide will not be glycosylated. The prokaryotic host selected for use herein must be compatible with the replicon and control sequences in the expression plasmid.

Suitable hosts may often include eukaryotic cells. Preferred eukaryotic hosts include, for example, yeast, fungi, insect cells, and mammalian cells either in vivo, or in tissue culture. Mammalian cells which may be useful as hosts include HeLa cells, cells of fibroblast origin such as VERO, 3T3 or CHOK1, HEK 293 cells or cells of lymphoid origin (such as 32D cells) and their derivatives. Preferred mammalian host cells include nonadherent cells such as CHO, 32D, and the like. Preferred yeast host cells include S. pombe, Pichia pastoris, S. cerevisiae (such as INVSc1), and the like.

In addition, plant cells are also available as hosts, and control sequences compatible with plant cells are available, such as the cauliflower mosaic virus 35S and 19S, nopaline synthase promoter and polyadenylation signal sequences, and the like. Another preferred host is an insect cell, for example the Drosophila larvae. Using insect cells as hosts, the Drosophila alcohol dehydrogenase or MT promoter can be used. Rubin, Science 240:1453-1459, 1988). Alternatively, baculovirus vectors can be engineered to express large amounts of peptide encoded by a desire gene sequence in insects cells (Jasny, Science 238:1653, 1987); Miller et al., In: Genetic Engineering (1986), Setlow, J. K., et al., eds., Plenum, Vol 8, pp. 277-297).

In a farther embodiment of the invention, there are provided libraries of expressible gene sequences produced by the methods of the invention. As shown in more detail in the Examples presented below, such libraries comprise gene sequences from a variety of sources such as yeast, mammals (including humans), and the like. The present invention also features the purified, isolated or enriched versions of the expressed gene products produced by the methods described above.

Kits comprising one or more containers or vials containing components for using the libraries of the present invention are also within the scope of the invention. Kits can comprise any one or more of the following elements: one or more expressible gene sequences, cells which are or can be transfected with said gene sequences, and antibodies recognizing the expressed gene product or an epitope tag associated therewith. Cells suitable for inclusion in such a kit include bacteria cells, yeast cells (such as INVSc1), insect cells or mammalian cells (such as CHO).

In one embodiment, such a kit can comprises a detergent solution, preferably the Trax® lysing reagent (6% NP-40 and 9% Triton X-100 in 1X PBS). Also included in the kit can be one or more binding partners, e.g., an antibody or antibodies, preferably a pair of antibodies to the same expressed gene product, which preferably do not compete for the same binding site on the expressed gene product.

In another embodiment, a kit can comprise more than one pair of such antibodies or other binding partners, each pair directed against a different target molecule, thus allowing the detection or measurement of a plurality of such target molecules in a sample. In a specific embodiment, one binding partner of the kit may be pre-adsorbed to a solid phase matrix, or alternatively, the binding partner and matrix are supplied separately and the attachment is performed as part of the assay procedure. The kit preferably contains the other necessary washing reagents well-known in the art. For EIA, the kit contains the chromogenic substrate as well as a reagent for stopping the enzymatic reaction when color development has occurred. The substrate included in the kit is one appropriate for the enzyme conjugated to one of the antibody preparations. These are well-known in the art, and some are exemplified below. The kit can optionally also comprise a target molecule standard; i.e., an amount of purified target molecule that is the target molecule being detected or measured.

In a specific embodiment, a kit of the invention comprises in one or more containers: (1) a solid phase carrier, such as a microtiter plate coated with a first binding partner; (2) a detectably labeled second binding partner which binds to the same expressed gene product as the first binding partner; (3) a standard sample of the expressed gene product recognized by the first and second binding partners; (4) concentrated detergent solution; and (5) optionally, diluent.

The invention will now be described in greater detail by reference to the following non-limiting examples.

### EXAMPLE 1

### High-throughput Expression of Yeast ORFs

The following example illustrates the creation of a library of expressible yeast gene sequences.

Amplification -

6,032 yeast ORFs and a corresponding gene-specific primer of the 3′ end of each were obtained from Research Genetics (Huntsville, Ala.) in a 96-well microtiter plate format at a concentration of 0.3 ng/μl Each gene specific primer was designed to exclude the gene's stop codon. Since the templates each contain a common sequence immediately 5′ of the start ATG (5′-GCAGTCCTGGAATTCCAGCTGACCACC) (SEQ ID NO:1), it was possible to amplify each template with a common 5′ primer.

5 μl of ORF template was added to a fresh 96-well microtiter plate (polycarbonate Thermowell Thinwall, Model M. Cat # 6511) using a 12 channel pipetter. 6 μl of specific 3′ primer solution (2 μM) was added and the total volume per well brought to 30 μl with PCR cocktail, immediately after which the plate was placed on ice. (PCR cocktail for 120 reactions- 720 μl 5X Buffer J, 48 μl dNTPs (50 mM stock), 12 μl common 5′ primer (1 μg/μl stock), 48 μl Taq DNA polymerase (Boeringer-Mannheim or Promega, 5 units/μl), 1.92 μl Pfu DNA polymerase (Stratgene, cat. # 600153-81, 2.5 units/μl) and 1464 μl distilled water. 5X Buffer J: 300 mM Tris (pH 9.5), 75 mM ammonium sulfate, 10 mM MgCl₂). The rubber Hybaid Micromat lid was washed by soaking in 0.1 M HCl, the rinsed for 2 minutes with distilled water and dried completely before applying to the 96-well plate.

The PCR reaction was performed using a Hybaid, Ltd. (Middlesex, UK) thermo-cycler according to the manufacturer's instructions. The conditions used were as follows: pre-melt step: 94° C.×4 min; melt step: 94° C.×30 sec, anneal step: 58° C.×45 sec, extend step: 72° C.×3 min—repeated for 25 cycles; final extension: 72° C.×4 min; final block temperature set to room temp (approx. 22° C.). The plates were stored at 40° C.

Purification -

The plates were spun briefly at 1000 rpm, then 10 μl of 6X gel loading dye was added to each well (6X gel loading dye: 6 mM Tris (pH 8), 6 mM EDTA, 0.03% Bromphenol Blue, 30% glycerol). The entire contents of each well were loaded onto a 1% low melt agarose (Invitrogen # 46-0150) gel (plus ethidium bromide at 20 μl of a 10 mg/ml solution added to 400 mls of agarose) in 1X TAE (50X TAE=242 g Tris base, 57.1 ml glacial acetic acid, 100 ml 0.5 M EDTA, pH 8.0 per liter (water)) and run at 110-120 volts for 1.25 to 1.5 hours. A UV light box was used to visualize the amplification products and ensure that only correct-sized PCR products are used in the insertion step.

Insertion into expression vector(s) -

The portion of each lane containing the amplified gene sequence was cut from the gel and transferred to a well in a 96-well microtiter plate, melted on a heat block (75° C.), and a portion of the melt multi-channel pipetted into a 96-well microtiter plate (7 μl/well) containing one of two expression vectors: TOPO-adapted pcDNA3.1/GS or pYES2/GS (Invitrogen, Carlsbad, Calif.) previously digested with HindIII. The plate was covered with parafilm and incubated at 37° C. for 7 minutes. Top 10 Chemically Competent Cells (Invitrogen) were added to each well (45 μl/well, O.D.=4.7), whereupon the plate was re-covered and incubated on ice for 5 minutes. The cells were then heat shocked on a 42° C. block for 1 minute and returned to ice for 1 minute. An aliquot of SOC medium was added to each well (150 μl, 20 g tryptone, 5 g yeast extract, 0.5 g NaCl, 250 mM KCl, 20 ml 1M glucose/liter), and the plate was incubated at 37° C. for 90 to 120 minutes.

The contents of each well were plated onto a LB(10 g tryptone, 5 g yeast extract, 10 g NaCl per liter)1.5% agar petrie plate containing the appropriate selection marker (ampicillin (50 μg/ml) for pYES2/GS and Zeocin™ (25 μg/ml) for pcDNA3.1/GS). The petrie plates were grown overnight at 37° C.

Verification of size and orientation -

Contamination is a potentially serious problem in this step. Care should be taken to guard against contaminating the process through airborne contamination, unsterile reagents or equipment, or well-to-well contamination.

Eight colonies were picked from each petrie plate and placed in eight individual wells of a 96-well microtiter plate. Each well contained 100 μl of 2X LB plus 100 μg/ml ampicillin or 50 μg/ml Zeocin™ as appropriate for the expression vector used. The plates were incubated overnight at 37° C.

The plates were spun briefly at 1000 rpm. The cells were stirred by pipetting up and down in a pipetter, then 2 μl from each well was transferred to a corresponding well in a PCR reaction plate containing 28 μl/well PCR cocktail (PCR cocktail for 840 reactions—5040 μl 5X Buffer J, 336 μl dNTPs (50 mM stock), 84 μl common 5′ primer (1 μg/μl stock, Dalton Chemical Lab. Inc, Ont. CAN), 84 μl 3′ H6stopprevu primer (1 μg/μl, Dalton Chemical Lab. Inc, Ont. CAN), 336 μl Taq DNA polymerase (Boeringer-Mannheim or Promega, 5 units/μl), and 17.64 mls distilled water. H6stopprevu primer has the sequence 5′ AAA CTC AAT GGT GAT GGT GAT GAT GACC-3′) (SEQ ID NO:2).

The PCR reaction was run essentially as described above with the following cycle: pre-melt step: 94° C.×10 min; melt step: 94° C.×1 min, anneal step: 67° C.×1 min, extend step: 72° C.×3 min—35 cycles; final extension: 72° C.×4 min; final block temp set to room temp (approximately 22° C.). The plates were spun briefly at 100 rpm and 6 μl of 6X gel loading dye added to each well. Samples were run on a 1% agarose gel which was subsequently stained with ethidium bromide. Only plasmids with correctly oriented inserts give an amplification product in this step.

The location of the positive clones was entered into a database and a spreadsheet of positive clones generated. The spreadsheet was downloaded onto a Qiagen BioRobot 9600™ to direct the re-racking of the positive cultures into deep-well culture blocks. Essentially, a single positive culture for each clone was grown and used to prepare plasmid DNA according to the Quia-Prep Turbo protocol.

CHO cells were transfected with the prepared plasmid DNA using the Pfx-6 PerFect Lipid system (Invitrogen, Cat #T930-16). Yeast cells (INVSc1) were transfected using the S.C. EasyComp Transformation kit (Invitrogen, Cat #K5050-01). Expression was verified by Western blot using anti-V5 antibody to detect the epitope tag. A total of 558 clones expressing a correct protein were obtained after a single pass.

### EXAMPLE 2

### High-throughput Expression of Human Gene Sequences

The following example illustrates the construction of a library of expressible human gene sequences using the method of the invention. Primers were constructed based on sequences of human genes available from GenBank.

Fetal human heart tissue was obtained from the International Institute for the Advancement of Medicine (IIAM). Poly A+nRNA was isolated using the FastTrack™ 2.0 Kit (Invitrogen, Carlsbad, Calif.) according to the manufacturer's instructions. The mRNA was converted to first-strand cDNA using a cDNA Cycle® Kit (Invitrogen) using the oligo dT primer provided and the protocols suggested. A single cDNA synthesis reaction was split into 12 separate wells of a 96-well PCR amplification plate, and PCR amplifications were performed using specific primer sets, essentially as described above, with the exception that the ratio of Taq to Pfu was 50:1 in the initial amplification (final conc. 2 U Taq:0.04 U Pfu/well).

Primers were synthesized using a Primerstation 960 (Intelligent Automation Systems, Inc.) used according to the manufacturer's instructions and were designed from sequences downloaded from Unigene and sent directly to the synthesizer. Approximately 15 nMoles of each primer, having an average length of 25 basepairs, was synthesized in a 96-well format. After synthesis, the primers were cleaved from the supports, deprotected and dried in the same 96-well format (see manufacturer's instructions).

The amplified gene sequences were purified and inserted into the pcDNA3.1/GS expression vector essentially as described above. The expression vectors containing sequences verified to be in the correct orientation were transfected into CHO cells in 96-well deep-well blocks using the Pfx-6 PerFect Lipid system (Invitrogen, Cat #T930-16) Cell lysates were made 48 hours after transfection, and the lysates were separated by SDS-PAGE and analyzed by Western blot according to standard protocols using an anti-V5 epitope tag Mab/horseradish peroxidase conjugate Table 1 lists the human proteins successfully expressed using this methodology. A total of 66 clones expressing a correct protein, out of 118, were obtained after a single pass.

TABLE 1
Human ORFs

| | | | Predicted | Actual |
|---|---|---|---|---|
| Plate Number | Accession Number | Description | Size | Size |
| M235 C7 | H-A06977 | albumin | 67.1 | 67.0 kDa |
| E1 | H-AB002391 | Human mRNA for KIAA0393 | 68.09 | 68 |
| | | gene, complete cds | | |
| H3 | H-AB006969 | Homo sapiens hGAA1 mRNA, | 68.42 | 70 |
| | | complete cds | | |
| E2 | H-AB007875 | Homo sapiens KIAA0415 mRNA, | 51.48 | 51 |
| | | complete cds | | |
| D1 | H-AB007887 | Homo sapiens KIAA0427 mRNA, | 66.55 | 70 |
| | | complete cds | | |
| M421 D6 | H-AB010710 | Homo sapiens mRNA for lectin- | 30.14 | 45.0 kDa |
| | | like oxidized LDL receptor, | | |
| | | complete cds | | |
| G3 | H-AD001528 | Homo sapiens spermidine | 40.37 | 40 |
| | | aminopropyltransferase mRNA, | | |
| | | complete cds | | |
| B5 | H-AE000659 | Homo sapiens T-cell receptor | 12.39 | 16 |
| | | alpha delta locus from bases | | |
| | | 250472 to 501670 (section 2 of 5) | | |
| | | of the C | | |
| E2 | H-AF004022 | Homo sapiens protein kinase | 38.28 | 44 |
| | | mRNA, complete cds | | |
| M428 C1 | H-AF004231 | Homo sapiens | 65.78 | 70.0 KDa |
| | | monocyte/macrophage Ig-related | | |
| | | receptor MIR-10 (MIR cl-10) | | |
| | | mRNA, complete cds | | |
| A5 | H-AF004327 | Homo sapiens angiopoietin-2 | 54.67 | 60 |
| | | mRNA, complete cds | | |
| C1 | H-AF006501 | Homo sapiens chromosome 22 | 14.08 | 24 |
| | | cosmid clone c1155, RNA | | |
| | | polymcrase II subunit 14.4 kDa | | |
| | | (POLRF) gene, complete cds | | |
| H4 | H-AF008936 | Homo sapiens syntaxin-16B | 35.75 | 47 |
| | | mRNA, complete cds | | |
| H5 | H-AF009243 | Homo sapiens proline-rich Gla | 22.33 | 36 |
| | | protein 2 (PRGP2) mRNA, | | |
| | | complete cds | | |
| M462 D6 | H-AF013249 | Homo sapiens leukocyte- | 31.68 | 40.0 kDa |
| | | associated Ig-like receptor-1 | | |
| | | (LAIR-1) mRNA, complete cds | | |
| A1 | H-AF013512 | untitled | 53.02 | 53 |
| A3 | H-AF013970 | Homo sapiens MTG8-like protein | 66.55 | 70 |
| | | (MTGRI) mRNA, complete cds | | |
| M467 A7 | H-AF014807 | Homo sapiens | 23.54 | 29.0 kDa |
| | | phosphatidylinositol synthase | | |
| | | (PIS) mRNA, complete cds | | |
| D2 | H-AF015257 | Homo sapiens flow-induced | 41.36 | 40 |
| | | endothelial G protein-coupled | | |
| | | receptor (FEG-1) mRNA, | | |
| | | complete cds | | |
| M422 B5 | H-AF017307 | Homo sapiens Ets-related | 40.92 | 49.0 kDa |
| | | transcription factor (ERT) mRNA, | | |
| | | complete cds | | |
| A6 | H-AF017656 | Homo sapiens G protein beta 5 | 38.94 | 48 |
| | | subunit mRNA, complete cds | | |
| E1 | H-AF017995 | Homo sapiens 3-phosphoinositide | 61.27 | 52 |
| | | dependent protein kinase-I | | |
| | | (PDKI) mRNA, complete cds | | |
| G1 | H-AF019612 | Homo sapiens S2P mRNA, | 57.2 | 57 |
| | | complete cds | | |
| D3 | H-AF020591 | Homo sapiens zinc finger protein | 78.76 | 74 |
| | | mRNA, complete cds | | |
| A7 | H-AF022385 | Homo sapiens apoptosis-related | 23.43 | 33 |
| | | protein TFAR15 (TFAR15) | | |
| | | mRNA, complete cds | | |
| H6 | H-AF024714 | Homo sapiens interferon- | 37.84 | 48 |
| | | inducible protein (AIM2) mRNA, | | |
| | | complete cds | | |
| B1 | H-AF025527 | Homo sapiens leucocyte | 48.4 | 47 |
| | | immunoglobulin-like receptor-4 | | |
| | | (LIR-4) mRNA, complete cds | | |
| M424 B4 | H-AF025532 | Homo sapiens leucocyte | 49.39 | 59.0 kDa |
| | | immunoglobulin-like receptor-5 | | |
| | | (LIR-5) mRNA, complete cds | | |
| H5 | H-AF026071 | Homo sapiens soluble death | 30.58 | 50 |
| | | receptor 3 beta (DR3) mRNA, | | |
| | | complete cds | | |
| M428 A1 | H-AF026273 | Homo sapiens interleukin-1 | 65.01 | 68.0 kDa |
| | | receptor-associated kinase-2 | | |
| | | mRNA, complete cds | | |
| B6 | H-AF026293 | Homo sapiens chaperonin | 58.96 | 58 |
| | | containing t-complex polypeptide | | |
| | | 1, beta subunit (Cctb) mRNA, | | |
| | | complete cds | | |
| B5 | H-AF026548 | Homo sapiens branched chain | 45.43 | 50 |
| | | alpha-ketoacid dehydrogenase | | |
| | | kinase precursor, mRNA, nuclear | | |
| | | gene encoding mitochondrial | | |
| | | protein, complete cds | | |
| B2 | H-AF027204 | Homo sapiens putative tetraspan | 21.78 | 27 |
| | | transmembrane protein L6H | | |
| | | (TM4SF5) mRNA, complete cds | | |
| M426 D3 | H-AF028008 | Homo sapiens SP1-like zinc | 56.43 | 64.0 kDa |
| | | finger transcription factor SLP | | |
| | | mRNA, complete cds | | |
| B1 | H-AF029232 | Homo sapiens calpamodulin | 70.62 | 70 |
| | | (CalpM) mRNA, complete cds | | |
| M422 A7 | H-AF029761 | Homo sapiens decoy receptor 2 | 42.57 | 50.0 kDa |
| | | mRNA, complete cds | | |
| M477 F3 | H-AF029893 | Homo sapiens i-beta-1,3-N- | 45.76 | 50.0 kDa |
| | | acetylglucosaminyltransferase | | |
| | | mRNA, complete cds | | |
| C5 | H-AF032437 | Homo sapiens mitogen activated | 51.92 | 50 |
| | | protein kinase activated protein | | |
| | | kinase gene, complete cds | | |
| M416 F3 | H-AF035824 | Homo sapiens vesicle soluble | 25.63 | 36.0 kDa |
| | | NSF attachment protein receptor | | |
| | | (VT11) mRNA, complete cds | | |
| F3 | H-AF037335 | Homo sapiens carbonic anhydrase | 39.05 | 39 |
| | | precursor (CA 12) mRNA, | | |
| | | complete cds | | |
| G1 | H-AF039019 | Homo sapiens zinc finger DNA | 87.45 | 87 |
| | | binding protein 89 kDa (ZBP-89) | | |
| | | mRNA, complete cds | | |
| G1 | H-AF039136 | Homo sapiens Fas binding protein | 81.51 | 98 |
| | | (hDaxx) mRNA, complete cds | | |
| A7 | H-AF040705 | Homo sapiens putative tumor | 31.57 | 41 |
| | | suppressor protein unspliced form | | |
| | | (Fus-2) mRNA, complete cds | | |
| M469 F1 | H-AF040958 | Homo sapiens lysosomal | 45.76 | 46.0 kDa |
| | | neuraminidase precursor, mRNA, | | |
| | | complete cds | | |
| G2 | H-AF043472 | Homo sapiens Shab-related | 54.12 | 64 |
| | | delayed-rectifier K+ channel | | |
| | | alpha subunit (Kv9.3) mRNA, | | |
| | | complete cds | | |
| E2 | H-AJ001340 | Homo sapiens mRNA for U3 | 52.36 | 60 |
| | | snoRNP associated 55 kDa | | |
| | | protein | | |
| G1 | H-D00096 | Transtyretin (prealbumin) | 16.28 | 20 |
| C4 | H-D00408 | Cytochrome P450 IIIA7 (P450- | 55.44 | 64 |
| | | HFLa) | | |
| M302 E7 | H-D00682 | cofilin | 18.37 | 30 |
| M383 G2 | H-D00726 | ferrochelatase | 46.64 | 50.0 kDa |
| M383 C3 | H-D00760 | proteasome, subunit HC3 | 25.85 | 34.0 kDa |
| M305 B4 | H-D00761 | proteasome, subunit HC5 | 26.62 | 33 |
| M266 F7 | H-D00763 | proteasome, subunit HC9 | 28.82 | 33 |
| E2 | H-D00860 | Phosphoribosyl pyrophosphate | 35.09 | 47 |
| | | synthetase subunit I | | |
| 215-13 | H-D10522 | human mRNA for 80K-L protein | 35 | 36.59 |
| M423 F5 | H-D11086 | Interleukin 2 receptor gamma | 40.7 | 45.0 kDa |
| | | chain | | |
| M248 D2 | H-D11094 | positive modulator of HIV tat- | 47.74 | 40.0 kDa |
| | | mediated transactivation | | |
| G3 | H-D11428 | Peripheral myelin protein 22 | 17.71 | 17 |
| M424 D3 | H-D13168 | Human gene for endothelin-B | 48.73 | 48.0 kDa |
| | | receptor (hET-BR) | | |
| M271 B8 | H-D13315 | glyoxalase 1, | 20.35 | 34.0 kDa |
| | | LACTOYLGLUTATHIONE | | |
| | | LYASE. CATALYZES THE | | |
| | | CONVERSION OF | | |
| | | HEMIMERCAPTAL, FORMED | | |
| | | FROM METHYLGLYOXAL | | |
| | | AND GLUTATHIONE, TO S- | | |
| | | LACTOYLGLUTATHIONE. | | |
| M306 F1 | H-D13627 | hypothetical protein | 60.39 | 90 |
| | | (GB: D13627) | | |
| M248 D1 | H-D13630 | hypothetical protein | 46.2 | 49 |
| | | (GB: D13630), Human mRNA for | | |
| | | KIAA0005 gene, complete cds | | |
| M270 D5 | H-D13634 | hypothetical protein | 34.65 | 42.0 kDa |
| | | (GB: D13634) | | |
| M250 D2 | H-D13642 | hypothetical protein | 44 | 48.0 kDa |
| | | (GB: D13642), Human mRNA for | | |
| | | KIAA0017 gene, complete cds | | |
| M250 E6 | H-D13748 | translation initiation factor 4A | 44.77 | 49.0 kDa |
| M305 C3 | H-D13892 | carboxyl methyltransferase, | 25.19 | 34 |
| | | aspartate | | |
| D1 | H-D13900 | enoyl-Coenzyme A hydratase, | 32.01 | 58 |
| | | short chain, mitochondrial | | |
| E1 | H-D14446 | Human HFREP-1 mRNA for | 34.43 | 40 |
| | | unknown protein, complete cds | | |
| 167-14 | H-D14497 | H. sapiens (Ewing's sarcoma cell | 51.44 | 64 |
| | | line) mRNA encoding open | | |
| | | reading frame | | |
| M266 D2 | H-D14520 | basic transcription element- | 24.2 | 33.0 kDa |
| | | binding protein 2 | | |
| M318 D2 | H-D14658 | hypothetical protein | 13.64 | 17 |
| | | (GB: D14658) | | |
| D2 | H-D14661 | Human mRNA for KIAA0105 | 16.72 | 28 |
| | | gene, complete cds | | |
| M236 E2 | H-D14662 | HYPOTHETICAL 29.5 KD | 24.75 | 36.0 kDa |
| | | PROTEIN IN UBPI3-KIPI | | |
| | | INTERGENIC REGION | | |
| | | [Saccharomyces cerevisiae] | | |
| M271 G6 | H-D14695 | hypothetical protein | 43.12 | 50.0 kDa |
| | | (GB: D14695), Human mRNA for | | |
| | | KIAA0025 gene, complete cds. | | |
| M311 A3 | H-D14696 | hypothetical protein | 25.74 | 30.0 kDa |
| | | (GB: D14696) | | |
| H3 | H-D14697 | Farnesyl diphosphate synthase | 46.2 | 55 |
| | | (farnesyl pyrophosphate | | |
| | | synthetase, | | |
| | | dimethylallyltranstransferase, | | |
| | | geranyltranstransferase) | | |
| M271 E7 | H-D14705 | catenin, alpha 2(E). Catenin | 99.77 | 110 |
| | | (cadherin-associated protein), | | |
| | | alpha 1 (102 kD). ASSOCIATES | | |
| | | WITH THE CYTOPLASMIC | | |
| | | DOMAIN OF A VARIETY OF | | |
| | | CADHERINS. | | |
| M236 A6 | H-D14811 | hypothetical protein | 30.25 | 42 |
| | | (GB: D14811) | | |
| M250 A3 | H-D14812 | hypothetical protein | | |
| | | (GB: D14812), Human mRNA for | | |
| | | KIAA0026 gene, complete cds | | |
| A5 | H-D14874 | Human mRNA for | 20.46 | 33 |
| | | adrenomedullin, complete cds | | |
| F3 | H-D14887 | Human mRNA for TFIIA-42, | 41.47 | 50 |
| | | complete cds | | |
| M250 H6 | H-D16234 | phospholipase C, alpha, | 55.66 | 56.0 kDa |
| | | PROBABLE PROTEIN | | |
| | | DISULFIDE ISOMERASE ER- | | |
| | | 60 PRECURSOR [Homo sapiens] | | |
| M305 B1 | H-D16480 | enoyl-CoA hydratase/3- | 84.04 | 84 |
| | | hydroxyacyl-CoA dehydrogenase | | |
| | | trifunctional protein, alpha- | | |
| | | subunit, mitochobdrial | | |
| M271 G2 | H-D16481 | 3-ketoacyl-CoA thiolase, beta | | |
| | | subunit, mitochodrial, | | |
| | | Hydroxyacyl-Coenzyme A | | |
| | | dehydrogenase/3-ketoacyl- | | |
| | | Coenzyme Athiolase/enoyl- | | |
| | | Coenzyme A hydratase | | |
| | | (trifunctional protein), beta | | |
| | | subunit | | |
| H1 | H-D16626 | Histidine ammonia-lyase | 72.38 | 64 |
| A2 | H-D17532 | Human mRNA for RCK, | 52.03 | 53 |
| | | complete cds | | |
| M266 F4 | H-D17554 | DNA-binding protein TAX | 31.79 | 38 |
| M248 A3 | H-D21235 | xeroderma pigmentosum group C | 40.04 | 55 |
| | | repair complementing protein | | |
| | | HHR23A | | |
| M235 E1 | H-D21261 | SM22-ALPHA HOMOLOG, | 22 | 31 |
| | | hypothetical protein | | |
| | | (GB: D21261) | | |
| M311 E1 | H-D21262 | hypothetical protein | 77.950 | 63 |
| | | (GB: D21262) | | |
| M466 B4 | H-D21853 | Human mRNA for KIAA0111 | 45.32 | 49.0 kDa |
| | | gene, complete cds | | |
| M311 H3 | H-D23660 | ribosomal protein L4 | 47.08 | 47 |
| M419 E1 | H-D26309 | human mRNA for LIMK (LIM | 71.240 | 75.0 kDa |
| | | kinase) | | |
| M271 B9 | H-D26362 | hypothetical protein | 79.97 | 70 |
| | | (GB: D26362), Human mRNA for | | |
| | | KIAA0043 gene, complete cds | | |
| M361 H2 | H-D26598 | proteasome, subunit HsC10-II | 22.66 | 33.0 kDa |
| M302 G4 | H-D26599 | proteasome, subunit HsC7-I | 22.22 | 34 |
| G1 | H-D26600 | Human mRNA for proteasome | 29.15 | 36 |
| | | subunit HsN3, complete cds | | |
| G9 | H-D28540 | hypothetical protein, CDC10 | 44.77 | 60 |
| | | homolog | | |
| M266 A5 | H-D29011 | proteasome, subunit X | 22.99 | 23 |
| M236 F3 | H-D29012 | Proteasome (prosome, macropain) | 26.4 | 32.0 kDa |
| | | delta subunit, beta type, 6 | | |
| C1 | H-D30037 | Human mRNA for | 29.92 | 38 |
| | | phosphatidylinositol transfer | | |
| | | protein (PI-TPbeta), complete cds | | |
| M250 H4 | H-D30655 | translation initiation factor 4AII, | 44.88 | 45.0 kDa |
| | | and ribosomal binding protein | | |
| 167-26 | H-D30742 | human mRNA for calmodulin- | 52.10 | 55 |
| | | dependent protein kinase IV | | |
| M236 A4 | H-D31767 | hypothetical protein | 18.59 | 30 |
| | | (GB: D31767), Human mRNA for | | |
| | | KIAA0058 gene, complete cds | | |
| E1 | H-D31883 | Human mRNA for KIAA0059 | 50.93 | 64 |
| | | gene, complete cds | | |
| G2 | H-D32129 | MHC class 1 protein HLA-A | 40.26 | 50 |
| M422 A6 | H-D37965 | Human mRNA for PDGF receptor | 41.36 | 45.0 kDa |
| | | beta-like tumor suppressor | | |
| | | (PRLTS), complete cds | | |
| M305 H4 | H-D38047 | 26S proteasome regulatory | 28.340 | 34.0 kDa |
| | | subunit P31 | | |
| M423 B2 | H-D38081 | Thromboxane A2 receptor | 37.84 | 45.0 kDa |
| M317 D3 | H-D38305 | ErbB-2 transducer | 38.06 | 49 |
| M270 A8 | H-D38583 | calgizzarin, Human mRNA for | 11.66 | 12 |
| | | calgizzarin, complete cds | | |
| M270 A6 | H-D42038 | hypothetical protein | 15.29 | 27 |
| | | (GB: D42038), Human mRNA for | | |
| | | KIAA0087 gene, complete cds | | |
| M318 F3 | H-D42085 | hypothetical protein | 90.2 | 100 |
| | | (GB: D42085) | | |
| M311 C2 | H-D43642 | YL-1 protein homolog | 40.15 | 36 |
| E1 | H-D45213 | Human mRNA for zinc finger | 12.87 | 20 |
| | | protein, complete cds | | |
| M236 B2 | H-D45248 | proteasome activator hPA28, | 26.4 | 38 |
| | | subunit beta, may be cell | | |
| | | adhesion protein | | |
| H3 | H-D45887 | Human mRNA for calmodulin, | 16.5 | 20 |
| | | complete cds | | |
| 166-3 | H-D45906 | human mRNA for LIMK-2 | 70 | 70.25 |
| A7 | H-D49357 | Human mRNA for S- | 43.56 | 51 |
| | | adenosylmethionine synthetase, | | |
| | | complete cds | | |
| C5 | H-D49489 | Human mRNA for protein | 48.51 | 54 |
| | | disulfide isomerase-related | | |
| | | protein P5, complete cds | | |
| M482 E2 | H-D49958 | Human fetus brain mRNA for | 30.69 | 32.0 kDa |
| | | membrane glycoprotein M6, | | |
| | | complete cds | | |
| M305 G5 | H-D50063 | proteasome, subunit p40 | 35.75 | 39 |
| M250 B6 | H-D50310 | cyclin 1, Human mRNA for cyclin | 41.58 | 47 |
| | | 1, complete cds | | |
| E3 | H-D50419 | Homo sapiens mRNA for OTK18, | 78.32 | 64 |
| | | complete cds | | |
| M298 B1 | H-D50495 | transcription elongation factor h- | 33 | 33.0 kDa |
| | | SII-T1 (GB: D50495) | | |
| M302 A3 | H-D50840 | ceramide glucosyltransferase | 43.45 | 44 |
| 167-40 | H-D50863 | human mRNA for TESK1 | 68.93 | 70 |
| 166-28 | H-D50927 | human myeloblast mRNA for | 60.46 | 64 |
| | | KIAA0137 gene | | |
| D1 | H-D63521 | Homo sapiens mRNA for LECT2 | 16.72 | 16 |
| | | precursor, complete cds | | |
| M302 A5 | H-D78134 | glycine-rich binding protein CIRP | 19.03 | 30.0 kDa |
| M313 E5 | H-D78275 | proteasome subunit p42 | 42.9 | 48.0 kDa |
| B3 | H-D79205 | Human mRNA for ribosomal | 5.72 | 10 |
| | | protein L39, complete cds | | |
| A4 | H-D79206 | Human gene for ryudocan core | 21.89 | 33 |
| | | protein, exon1-5, complete cds | | |
| A1 | H-D80008 | Human mRNA for KIAA0186 | 21.67 | 32 |
| | | gene, complete cds | | |
| M298 H4 | H-D83004 | ubiquitin-conjugating enzyme E2 | 16.83 | 32.0 kDa |
| | | similar to Drosophila bendless | | |
| | | gene product | | |
| C3 | H-D83702 | Human brain mRNA for | 64.57 | 64 |
| | | photolyase homolog, complete | | |
| | | cds | | |
| M306 A1 | H-D83735 | neutral calponin | 34.1 | 34.0 kDa |
| H2 | H-D86322 | Homo sapiens mRNA for | 67.21 | 64 |
| | | calmegin, complete cds | | |
| B1 | H-D86979 | Human mRNA for KIAA0226 | 82.22 | 82 |
| | | gene, complete cds | | |
| 169-16 | H-D87116 | dual specificity mitogen-activated | 38.24 | 42 |
| | | protein kinase kinase 3 | | |
| 166-27 | H-D87119 | human cancellous bone osteoblast | 37.80 | 40 |
| | | mRNA for GS3955 | | |
| E2 | H-D88308 | Homo sapiens mRNA for very- | 68.31 | 64 |
| | | long-chain acyl-CoA synthetase, | | |
| | | complete cds | | |
| 166-26 | H-D89077 | human mRNA for Src-like | 30.43 | 38 |
| | | adapter protein | | |
| M440 H2 | H-D89479 | Homo sapiens mRNA for STIB2, | 32.67 | 38.0 kDa |
| | | complete cds | | |
| H1 | H-D90086 | Human pyruvate dehydrogenase | 39.6 | 35 |
| | | (EC 1.2.4.1) beta subunit gene, | | |
| | | exons 1-10 | | |
| M362 F1 | H-D90209 | DNA-binding protein | 38.72 | 48.0 kDa |
| | | TAXREB67 | | |
| M316 B2 | H-J00068 | actin, alpha 1, skeletal muscle | 41.58 | 50 |
| M250 B2 | H-J00194 | major histocompatibility complex, | 28.05 | 36.0 kDa |
| | | MHC class II, DR alpha | | |
| G2 | H-J00212 | Interferon, alpha 21 | 20.9 | 30 |
| G1 | H-J00287 | Human pepsinogen gene | 42.79 | 48 |
| M298 C2 | H-J02611 | apolipoprotein D | 20.9 | 31.0 kDa |
| M266 C4 | H-J02683 | ADP/ATP carrier protein | 32.89 | 36 |
| M383 H2 | H-J02685 | plasminogen activator inhibitor, | 45.76 | 50.0 kDa |
| | | placenta | | |
| 167-3 | H-J02853 | “casein kinase II, alpha chain” | 43.08 | 50 |
| E3 | H-J02854 | Human 20-kDa myosin light | 19.03 | 31 |
| | | chain (MLC-2) mRNA, complete | | |
| | | cds | | |
| M248 F3 | H-J02874 | fatty-acid-binding protein 4, | 14.63 | 17 |
| | | adipocyte, LIPID TRANSPORT | | |
| | | PROTEIN IN ADIPOCYTES | | |
| M235 D5 | H-J02939 | antigen 4F2, heavy chain | 58.3 | 58 |
| C3 | H-J02943 | Corticosteroid binding globulin | 44.66 | 50 |
| M248 F2 | H-J02966 | adenine nucleotide translocator 1 | 32.78 | 33 |
| | | (skeletal muscle) [ANT1], | | |
| | | CATALYZES THE EXCHANGE | | |
| | | OF ADP AND ATP ACROSS | | |
| | | THE MITOCHONDRIAL | | |
| | | INNER MEMBRANE. | | |
| E1 | H-J02982 | Glycophorin B | 10.12 | 20 |
| 167-91 | H-J03075 | “protein kinase c substrate, 80 kD | 58.04 | 98 |
| | | protein heavy chain” | | |
| M266 A3 | H-J03191 | profilin 1 | 15.51 | 17.0 kDa |
| M248 H4 | H-J03231 | glucose-6-phosphate | 56.76 | 51 |
| | | dehydrogenase [G6PD] | | |
| M266 F2 | H-J03459 | LEUKOTRIENE A-4 | 67.32 | 64 |
| | | HYDROLASE [Homo sapiens] | | |
| A2 | H-J03460 | Prolactin-induced protein | 16.17 | 26 |
| M271 E5 | H-J03799 | laminin receptor 1, Laminin | 32.56 | |
| | | receptor (2H5 epitope). 40S | | |
| | | RIBOSOMAL PROTEIN SA | | |
| | | [Homo sapiens]. | | |
| M440 A4 | H-J03890 | Human pulmonary surfactant | 21.78 | 30.0 kDa |
| | | protein C (SP-C) and pulmonary | | |
| | | surfactant protein C1 (SP-C1) | | |
| | | genes, complete cds | | |
| M271 D8 | H-J03934 | NAD(P)H menadione | 30.25 | 38 |
| | | oxidoreductase 1, dioxin- | | |
| | | inducible. INVOLVED IN | | |
| | | DETOXICATION PATHWAYS. | | |
| M271 A8 | H-J04031 | trifunctional enzyme | 102.96 | 117.0 kDa |
| | | (GB: J04031). C-1- | | |
| | | TETRAHYDROFOLATE | | |
| | | SYNTHASE, CYTOPLASMIC | | |
| | | [Homo sapiens] | | |
| M305 F6 | H-J04046 | calmodulin 3 [CALM3] | 16.5 | 20 |
| M305 G7 | H-J04071 | cytotoxic T-lymphocyte- | 27.28 | 38 |
| | | associated serine esterase 1 | | |
| | | (cathepsin G-like 1, granzyme B) | | |
| | | [CTLA1] | | |
| M311 D2 | H-J04183 | lysosomal-associated membrane | 44.99 | 47 |
| | | protein 2 | | |
| M300 F4 | H-J04205 | Sjogren syndrome antigen B | 44.99 | 51.0 kDa |
| M416 G8 | H-J04430 | Acid phosphatase 5, tartrate | 35.64 | 45.0 kDa |
| | | resistant | | |
| B1 | H-J04501 | Glycogen synthase 1 (muscle) | 81.18 | 81 |
| M313 B5 | H-J04543 | synexin | 51.37 | 51 |
| B1 | H-J04605 | Peptidase D | 54.34 | 55 |
| M250 C6 | H-J04615 | small nuclear ribonucleoprotein | 26.51 | 34.0 kDa |
| | | SM-D, ROLE IN THE PRE- | | |
| | | mRNA SPLICING OR IN | | |
| | | SNRNPSTRUCTURE. | | |
| M248 E2 | H-J04964 | steroid sulfatase (microsomal) | 64.24 | 60.0 kDa |
| | | [STS] | | |
| M250 A7 | H-J05249 | replication protein A, 32 kDa | 29.81 | 36.0 kDa |
| | | subunit, REQUIRED FOR SV | | |
| | | 40 DNA REPLICATION IN | | |
| | | VITRO, RP-A IS SINGLE- | | |
| | | STRANDED DNA-BINDING | | |
| | | PROTEIN. | | |
| F1 | H-J05272 | IMP (inosine monophosphate) | 56.65 | 51 |
| | | dehydrogenase 1 | | |
| 169-15 | H-J05401 | “creatine kinase, sarcomeric | 50 | 46.16 |
| | | mitochondrial precursor” | | |
| M266 E4 | H-J05448 | RNA polymerase II, subunit B33 | 30.36 | 35.0 kDa |
| M305 C2 | H-K00558 | tubulin, alpha k1 [TUBA*] | 49.72 | 52.0 kDa |
| M416 H7 | H-K01571 | Human T-cell receptor active | 34.43 | 36.0 kDa |
| | | beta-chain, mRNA from cell line | | |
| | | MOLT-3, complete cds | | |
| M311 E4 | H-K01763 | haptoglobin | 38.28 | 47.0 kDa |
| G5 | H-K02100 | Human omithine | 39.05 | 47 |
| | | transcarbamylase (OTC) mRNA, | | |
| | | complete coding sequence | | |
| M302 D5 | H-K02574 | purine nucleoside phosphorylase | 31.9 | 36.0 kDa |
| 169-39 | H-K02581 | “thymidine kinase, cytosolic” | 34 | 25.81 |
| M248 E4 | H-K03020 | phenylalanine hydroxylase [PAH] | 49.83 | 50 |
| M556 B3 | H-K03191 | Cytochrome P450, subfamily 1 | 56.43 | 53.0 kDa |
| | | (aromatic compound-inducible), | | |
| | | polypeptide 1 | | |
| H2 | H-L00190 | Antithrombin III | 51.15 | 55 |
| 169-62 | H-L01087 | “protein kinase c, theta type” | 80 | 77.7 |
| M318 C2 | H-L01124 | ribosomal protein S13 | 16.72 | 28 |
| M313 F1 | H-L02321 | glutathione S-transferase M5 | 24.09 | 28 |
| M305 E5 | H-L02426 | protease 26S, regulatory subunit 4 | 48.51 | 53 |
| M302 D4 | H-L02547 | cleavage stimulation factor, 50 kDa | 47.52 | 50.0 kDa |
| | | subunit | | |
| M266 H7 | H-L02648 | transcobalamin II | 47.08 | 48.0 kDa |
| E2 | H-L02932 | Human peroxisome proliferator | 51.59 | 59 |
| | | activated receptor mRNA, | | |
| | | complete cds | | |
| M270 A1 | H-L03380 | gonadotropin-releasing hormone | 36.19 | 36 |
| | | receptor [GRHR]. THIS | | |
| | | RECEPTOR MEDIATES ITS | | |
| | | ACTION BY ASSOCIATION | | |
| | | WITH G PROTEINS | | |
| M270 H1 | H-L03411 | RD protein [RDBP], Radin blood | 41.91 | 59.0 kDa |
| | | group | | |
| D3 | H-L03426 | Human XE7 mRNA, complete | 42.46 | 45 |
| | | alternate coding regions | | |
| B1 | H-L03785 | Myosin, light polypeptide 5, | 19.14 | 32 |
| | | regulatory | | |
| A7 | H-L04483 | ribosomal protein S21 | 9.24 | 34 |
| M416 B2 | H-L05147 | Human dual specificity | 20.46 | 30.0 kDa |
| | | phosphatase tyrosine/serine | | |
| | | mRNA, complete cds | | |
| 215-38 | H-L05624 | dual specificity mitogen-activated | 50 | 43.30 |
| | | protein kinase kinase 1 | | |
| M271 D4 | H-L06132 | anion channel, voltage-gated, | 31.24 | 37 |
| | | isoform 1. FORMS A CHANNEL | | |
| | | THROUGH THE CELL | | |
| | | MEMBRANE, THAT ALLOWS | | |
| | | DIFFUSION FROM SMALL | | |
| | | HYDROPHYLIC MOLECULES. | | |
| 169-27 | H-L06139 | tyrosine-protein kinase receptor | 125 | 123.7 |
| | | TIE-2 precursor | | |
| H1 | H-L06147 | Human (clone SY11) golgin-95 | 68.31 | 68 |
| | | mRNA, complete cds | | |
| M250 A1 | H-L06419 | procollagen-lysine, 2-oxoglutarate | 80.08 | 80.0 kDa |
| | | 5-dioxygenase (lysine | | |
| | | hydroxylase) [PLOD] | | |
| M236 F6 | H-L06498 | ribosomal protein S20 | 13.2 | 23.0 kDa |
| M318 D1 | H-L06499 | ribosomal protein L37a | 10.23 | 27 |
| M270 D1 | H-L07414 | CD40 antigen ligand [CD40LG], | 28.82 | 36 |
| | | NVOLVED IN | | |
| | | IMMUNOGLOBULIN CLASS | | |
| | | SWITCHING. | | |
| M298 A6 | H-L07548 | aminoacylase 1 | 44.99 | 52.0 kDa |
| M424 C3 | H-L07592 | Human peroxisome proliferator | 48.62 | 48.0 kDa |
| | | activated receptor mRNA, | | |
| | | complete cds | | |
| M298 G6 | H-L07633 | proteasome (prosome, macropain) | 27.5 | 33.0 kDa |
| | | activator subunit 1 (PA28 alpha) | | |
| | | [PSME1] | | |
| M318 B1 | H-L08096 | CD70 antigen (CD27 ligand) | 21.34 | 28 |
| | | [CD70] | | |
| D2 | H-L08187 | cytokine receptor EBI3 | 25.3 | 42 |
| M313 F4 | H-L08850 | amyloid, non-A beta component, | 15.51 | 31.0 kDa |
| | | Alzheimer's disease | | |
| M426 E1 | H-L08895 | MADS box transcription enhancer | 52.14 | 60.0 kDa |
| | | factor 2, polypeptide C (myocyte | | |
| | | enhancer factor 2C) | | |
| M266 A8 | H-L09235 | ATPase, vacuolar | 67.98 | 64.0 kDa |
| M266 D1 | H-L09604 | differentiation-dependent | 16.83 | 17.0 kDa |
| | | intestinal membrane A4 protein | | |
| | | (Homo sapiens) | | |
| M317 C1 | H-L10338 | sodium channel, voltage-gated, | 24.09 | 24 |
| | | type I, beta polypeptide [SCN1B] | | |
| M317 E1 | H-L10717 | tyrosine-protein kinase ITK/TSK | 68.270 | 68.0 kDa |
| M300 B5 | H-L10820 | formyl peptide receptor 1 [FPR1] | 38.61 | 37 |
| M312 A4 | H-L10838 | pre-mRNA splicing factor SRp20 | 18.15 | 31.0 kDa |
| M300 A5 | H-L10918 | chemokine (C-C) receptor 1 | 39.16 | 30 |
| | | [CMKBR1] | | |
| M311 F2 | H-L11245 | complement component 4-binding | 27.83 | 30 |
| | | protein, beta | | |
| M266 B7 | H-L11353 | neurofibromatosis 2 (bilateral | 65.56 | 63.0 kDa |
| | | acoustic neuroma) [NF2] | | |
| M311 B3 | H-L11667 | cyclophilin 40 | 40.81 | 50.0 kDa |
| 215-49 | H-L11695 | serine/threonine-protein kinase | 64 | 55.40 |
| | | receptor R4 precursor | | |
| M466 C2 | H-L11931 | Human cytosolic serine | 53.24 | 56.0 kDa |
| | | hydroxymethyltransferase | | |
| | | (SHMT) mRNA, complete cds | | |
| M271 B7 | H-L12168 | ADENYLYL CYCLASE- | 52.36 | 60.0 kDa |
| | | ASSOCIATED PROTEIN 1 | | |
| | | [Homo sapiens] | | |
| M416 D4 | H-L12964 | Interleukin-activated receptor, | 28.16 | 38.0 kDa |
| | | homolog of mouse Ly63 | | |
| B3 | H-L13203 | Human HNF-3/fork-head | 38.72 | 49 |
| | | homolog-3 HFH-3 mRNA, | | |
| | | complete cds | | |
| D2 | H-L13744 | Human AF-9 mRNA, complete | 62.59 | 63 |
| | | cds | | |
| 167-8 | H-L13943 | glycerol kinase | 60 | 57.71 |
| M311 G3 | H-L13974 | leucine zipper protein | 41.14 | 51 |
| | | (GB: L13974) | | |
| M271 H5 | H-L13977 | LYSOSOMAL PRO-X | 54.67 | 57 |
| | | CARBOXYPEPTIDASE | | |
| | | PRECURSOR [Homo sapiens]. | | |
| M270 G2 | H-L14283 | protein kinase C, zeta [PRKCZ], | 65.23 | 98 |
| | | SERINE-AND THREONINE- | | |
| | | SPECIFIC ENZYME. | | |
| M235 A3 | H-L14286 | antioxidant protein, thiol-specific | 21.89 | 32.0 kDa |
| M426 H3 | H-L14778 | Protein phosphatase 3 (formerly | 57.42 | 60.0 kDa |
| | | 2B), catalytic subunit, alpha | | |
| | | isoform (calcineurin A | | |
| | | alpha) {alternative products} | | |
| B4 | H-L15702 | complement factor B | 84.15 | 100 |
| M426 A4 | H-L16794 | Human-transcription factor | 57.42 | 60.0 kDa |
| | | (MEF2) mRNA, complete cds | | |
| 215-25 | H-L16862 | g protein-coupled receptor kinase | 70 | 63.4 |
| | | GRK6 | | |
| 167-74 | H-L16991 | thymidylate kinase | 36 | 23.39 |
| 169-3 | H-L18964 | “protein kinase c, iota type” | 80 | 64.64 |
| M305 E2 | H-L18972 | hypothetical protein (GB: L18972) | 75.24 | 78 |
| M426 D4 | H-L19067 | Human NF-kappa-B transcription | 59.18 | 63.0 kDa |
| | | factor p65 subunit mRNA, | | |
| | | complete cds | | |
| 215-26 | H-L19268 | Homo sapiens myotonic | 70 | 68.71 |
| | | dystrophy associated protein | | |
| | | kinase mRNA | | |
| M271 E1 | H-L19297 | carbonic anhydrase V [CA5], | 33.66 | 42 |
| | | Mitochondrial carbonic | | |
| | | anhydrase. REVERSIBLE | | |
| | | HYDRATATION OF CARBON | | |
| | | DIOXIDE. | | |
| M298 G4 | H-L19437 | transaldolase | 37.18 | 39.0 kDa |
| M423 C4 | H-L19593 | Interleukin 8 receptor, beta | 39.71 | 41.0 kDa |
| G1 | H-L19686 | Homo sapiens macrophage | 12.76 | 13 |
| | | migration inhibitory factor (MIF) | | |
| | | gene, complete cds | | |
| G2 | H-L19739 | metallopanstimulin 1 | 9.35 | 32 |
| M302 E3 | H-L19871 | activating transcription factor 3 | 20.02 | 36.0 kDa |
| 167-86 | H-L20422 | 14-3-3 protein eta | 34 | 27.13 |
| M440 B2 | H-L20492 | Human gamma-glutamyl | 24.86 | 35.0 kDa |
| | | transpeptidase mRNA, complete | | |
| | | cds | | |
| M315 B1 | H-L20688 | GDP-dissociation inhibitor | 22.22 | 32 |
| | | protein rhoA | | |
| M271 H3 | H-L20941 | ferritin, heavy polypeptide. | 20.24 | 32 |
| | | FERRITIN IS AN | | |
| | | INTRACELLULAR | | |
| | | MOLECULE THAT STORES | | |
| | | IRON IN A SOLUBLE, | | |
| | | NONTOXIC, READILY | | |
| | | AVAILABLE FORM. | | |
| M235 B7 | H-L21893 | Na+/taurocholate cotransporter, | | |
| | | STRICTLY DEPENDENT ON | | |
| | | THE | | |
| F1 | H-L21934 | Sterol O-acyltransferase (acyl- | 60.61 | 60 |
| | | Coenzyme A: cholesterol | | |
| | | acyltransferase) | | |
| C2 | H-L22075 | Human guanine nucleotide | 41.58 | 50 |
| | | regulatory protein (G13) mRNA, | | |
| | | complete cds | | |
| 169-18 | H-L22206 | vasopressin v2 receptor | 60 | 58.00 |
| M421 A10 | H-L22214 | Human adenosine A1 receptor | 35.97 | 38.0 kDa |
| | | (ADORAI) mRNA exons 1-6, | | |
| | | complete cds | | |
| M424 F1 | H-L23959 | Homo sapiens E2F-related | 45.21 | 53.0 kDa |
| | | transcription factor (DP-1) | | |
| | | mRNA, complete cds | | |
| C2 | H-L24498 | Human gadd45 gene, complete | 18.26 | 28 |
| | | cds | | |
| M302 E2 | H-L25080 | proto-oncogene rhoA, multidrug | 21.34 | 31 |
| | | resistance protein | | |
| M270 B8 | H-L25081 | guanine nucleotide-binding and | 21.34 | 30 |
| | | transforming protein rhoC, | | |
| | | Aplysia ras-related homolog 9 | | |
| M236 E3 | H-L25085 | Sec61 complex, beta subunit, | 10.67 | 19 |
| | | PROTEIN TRANSLOCATION | | |
| | | IN THE ENDOPLASMIC | | |
| | | RETICULUM | | |
| 167-85 | H-L25610 | cyclin-dependent kinase inhibitor 1 | 32 | 18.11 |
| B2 | H-L25610 | cyclin-dependent kinase inhibitor 1 | 18.110 | 40 |
| M297 H2 | H-L26232 | cathepsin A/phospholipid transfer | 54.34 | 64.0 kDa |
| | | protein | | |
| 167-4 | H-L26318 | stress-activated protein kinase | 52 | 42.31 |
| | | JNK1 | | |
| M428 F1 | H-L27586 | Human TR4 orphan receptor | 67.76 | 67.0 kDa |
| | | mRNA, complete cds | | |
| M302 E5 | H-L27711 | protein phosphatase KAP1 | 23.43 | 28 |
| M250 A6 | H-L28010 | Homo sapiens HnRNP F protein | | |
| | | mRNA, complete cds, | | |
| F1 | H-L28821 | Alpha mannosidase II isozyme | 87.67 | 87 |
| 167-89 | H-L28824 | tyrosine-protein kinase SYK | 70 | 69.92 |
| M298 E6 | H-L28997 | ADP-ribosylation factor-like | 20.02 | 33.0 kDa |
| | | gene 1 | | |
| D4 | H-L29219 | Homo sapiens clk 1 mRNA, | 53.35 | 60 |
| | | complete cds | | |
| 169-63 | H-L29222 | Homo sapiens clk 1 mRNA | 25 | 15.03 |
| M429 B3 | H-L29277 | Signal transducer and activator of | 84.81 | 88.0 kDa |
| | | transcription 3 (acute-phase | | |
| | | response factor) | | |
| C1 | H-L29433 | Human factor X (blood | 53.79 | 64 |
| | | coagulation factor) gene | | |
| G3 | H-L31860 | Glycophorin A | 16.61 | 26 |
| D1 | H-L31881 | Nuclear factor 1/X (CCAAT- | 48.62 | 48 |
| | | binding transcription factor) | | |
| 169-13 | H-L31951 | human protein kinase (JNK2) | 55 | 46.71 |
| | | mRNA | | |
| A1 | H-L32179 | Arylacetamide deacetylase | 44 | 50 |
| | | (esterase) | | |
| B2 | H-L33404 | Human stratum corneum | 27.94 | 36 |
| | | chymotryptic enzyme mRNA, | | |
| | | complete cds | | |
| M312 D3 | H-L33799 | procollagen C-proteinase | 49.5 | 51.0 kDa |
| | | enhancer | | |
| 169-77 | H-L33801 | human protein kinase mRNA | 55 | 46.27 |
| | | GSK-3 | | |
| M305 D6 | H-L34041 | L-glycerol-3-phosphate: NAD+ | 38.5 | 42.0 kDa |
| | | oxidoreductase | | |
| B4 | H-L34355 | Homo sapiens (clone p4) 50 kD | 42.68 | 47 |
| | | dystrophin-associated | | |
| | | glycoprotein mRNA, complete | | |
| | | cds | | |
| M297 B3 | H-L35013 | spliceosomal protein SAP 49 | 46.75 | 52.0 kDa |
| 167-32 | H-L35253 | human CSaids binding protein | 52 | 39.67 |
| | | (CSBP1) mRNA | | |
| M266 D6 | H-L35545 | C/activated protein C receptor, | 26.29 | 38.0 kDa |
| | | endothelial | | |
| M300 F1 | H-L35594 | autotaxin | 100.76 | 91.0 kDa |
| M318 E2 | H-L36720 | bystin | 33.77 | 29 |
| M305 H2 | H-L37127 | RNA polymerase II | 12.98 | 16 |
| M300 D1 | H-L38490 | ADP-ribosylation factor | 22.22 | 32 |
| | | (GB: L38490) | | |
| M318 E1 | H-L38941 | ribosomal protein L34 | 12.98 | 18 |
| C2 | H-L38969 | Homo sapiens thrombospondin 3 | 105.27 | 110 |
| | | (THBS3) gene, complete cds | | |
| M476 F4 | H-L39060 | Homo sapiens transcription factor | 49.61 | 53.0 kDa |
| | | SL1 mRNA, complete cds | | |
| M300 E4 | H-L40399 | hypothetical protein (GB: L40399) | 29.26 | 36 |
| E3 | H-L40802 | Homo sapiens 17-beta- | 42.68 | 60 |
| | | hydroxysteroid dehydrogenase | | |
| | | (17-HSD) gene | | |
| M478 F1 | H-L40904 | H. sapiens peroxisome | 52.69 | 60.0 kDa |
| | | proliferator activated receptor | | |
| | | gamma, complete cds | | |
| M306 C2 | H-L41268 | natural killer associated transcript | 37.62 | 40 |
| | | 2 [NKAT2*] | | |
| M306 E2 | H-L41270 | natural killer associated transcript | 50.16 | 65.0 kDa |
| | | 4 [NKAT4*] | | |
| M306 F2 | H-L41347 | natural killer associated transcript | 33.55 | 40 |
| | | 5 [NKAT5*] | | |
| M468 C3 | H-L41351 | Homo sapiens prostasin mRNA, | 37.84 | 45.0 kDa |
| | | complete cds | | |
| 169-53 | H-L41816 | Homo sapiens cam kinase 1 | 48 | 40.77 |
| | | mRNA | | |
| 167-25 | H-L41939 | tyrosine-protein kinase receptor | 108 | 108.6 |
| | | EPH-3 precursor | | |
| C3 | H-L42374 | Homo sapiens protein | 54.78 | 64 |
| | | phosphatase 2A B56-beta (PP2A) | | |
| | | mRNA, complete cds | | |
| M306 B1 | H-L42531 | glutathione synthetase | 52.25 | 54.0 kDa |
| M302 F6 | H-L42856 | RNA polymerase II transcription | 13.09 | 20.0 kDa |
| | | factor SIII, p18 subunit | | |
| M313 C7 | H-L76200 | guanylate kinase (GUK1) | 21.78 | 32.0 kDa |
| M428 E1 | H-L76702 | Homo sapiens protein | 66.33 | 68.0 kDa |
| | | phosphatase 2A B56-delta (PP2A) | | |
| | | mRNA, complete cds | | |
| M478 A1 | H-L76703 | Homo sapiens protein | 51.48 | 60.0 kDa |
| | | phosphatase 2A B56-epsilon | | |
| | | (PP2A) mRNA, complete cds | | |
| 166-52 | H-L77213 | H. sapiens phosphomevalonate | 34 | 21.19 |
| | | kinase mRNA | | |
| 169-64 | H-L77964 | H. sapiens ERK3 mRNA | 100 | 79.38 |
| M360 C3 | H-M10050 | fatty-acid-binding protein 2, | 14.08 | 20.0 kDa |
| | | intestinal | | |
| D5 | H-M10050 | fatty-acid-binding protein 2, | 14.08 | 36 |
| | | intestinal | | |
| M421 E7 | H-M10058 | Asialoglycoprotein receptor 1 | 32.12 | 48.0 kDa |
| M429 D3 | H-M10901 | Glucocorticoid receptor | 85.58 | 85.0 kDa |
| M312 G1 | H-M11025 | asialoglycoprotein receptor 2 | 34.32 | 34.0 kDa |
| 167-44 | H-M11026 | interferon alpha-4 precursor | 33 | 20.86 |
| F2 | H-M11321 | Human group-specific component | 52.25 | 56 |
| | | vitamin D-binding protein | | |
| | | mRNA, complete cds | | |
| M236 B5 | H-M11354 | histone H3.2, CENTRAL ROLE | 15.07 | 24 |
| | | IN NUCLEOSOME | | |
| | | FORMATION | | |
| M236 G2 | H-M11433 | retinol-binding protein 1, cellular | 14.96 | 28 |
| | | transport protein | | |
| M270 G7 | H-M11560 | aldolase A, FRUCTOSE- | 40.15 | 40 |
| | | BISPHOSPHATE ALDOLASE A | | |
| | | [Homo sapiens] | | |
| H3 | H-M11717 | Human heat shock protein (hsp | 70.51 | 60 |
| | | 70) gene, complete cds | | |
| E1 | H-M12523 | Human serum albumin (ALB) | 67.1 | 70 |
| | | gene, complete cds | | |
| B5 | H-M12963 | Alcohol dehydrogenase 1 (class | 41.36 | 48 |
| | | 1), alpha polypeptide | | |
| D6 | H-M13228 | | 51.15 | 50 |
| D4 | H-M13981 | Inhibin, alpha | 40.37 | 50 |
| M236 G4 | H-M13982 | interleukin 4 [IL4] precursor, B- | 16.94 | 30 |
| | | cell activator | | |
| M271 B6 | H-M14043 | lipocortin II, Annexin II | 37.4 | 45.0 kDa |
| | | (lipocortin II). CALCIUM- | | |
| | | REGULATED MEMBRANE- | | |
| | | BINDING PROTEIN | | |
| M271 F4 | H-M14218 | argininosuccinate lyase | 51.04 | 56 |
| M297 A3 | H-M14221 | cathepsin B | 37.4 | 32.0 kDa |
| M305 B2 | H-M14328 | enolase, alpha | 47.85 | 50 |
| 167-54 | H-M14333 | human c-syn protooncogene | 60 | 59.14 |
| 167-51 | H-M14505 | H. sapiens mRNA (open reading | 36 | 33.40 |
| | | frame; patient SK29(AV)) | | |
| 215-74 | H-M14676 | human src-like kinase (slk) | 60 | 59.14 |
| | | mRNA | | |
| 167-55 | H-M14780 | “creatine kinase, m chain” | 52 | 41.98 |
| M416 F8 | H-M15059 | Fc fragment of IgE, low affinity | 35.42 | 45.0 kDa |
| | | II, receptor for (CD23A) | | |
| M271 F1 | H-M15182 | glucuronidase, beta [GUSB], | 71.72 | 72 |
| | | PLAYS AN IMPORTANT ROLE | | |
| | | IN THE DEGRADATION OF | | |
| | | DERMATAN AND KERATAN | | |
| | | SULFATES. | | |
| 215-37 | H-M15465 | human pyruvate kinase type L | 64 | 59.80 |
| | | mRNA | | |
| M298 A4 | H-M15796 | cyclin | 28.82 | 43.0 kDa |
| C3 | H-M15800 | Mal, T-cell differentiation protein | 16.94 | 17 |
| M440 E1 | H-M15841 | Human U2 small nuclear RNA- | 24.86 | 34.0 kDa |
| | | associated B antigen mRNA, | | |
| | | complete cds | | |
| M248 C3 | H-M15887 | endozepine | 9.68 | 15.0 kDa |
| M463 A2 | H-M15990 | human c-yes-1 mRNA | 59.800 | 65.0 kDa |
| M418 E2 | H-M16038 | tyrosine-protein kinase LYN | 56.390 | 64.0 kDa |
| M266 D3 | H-M16342 | HETEROGENEOUS NUCLEAR | 32.01 | 49 |
| | | RIBONUCLEOPROTEINS | | |
| | | C1/C2 [Homo sapiens]; small | | |
| | | nuclear ribonucleoprotein, | | |
| | | polypeptide C | | |
| 167-20 | H-M16591 | tyrosine-protein kinase HCK | 60 | 55.62 |
| C7 | H-M16591 | tyrosine-protein kinase HCK | 55.620 | 70 |
| M305 E7 | H-M16660 | heat shock 90 kD protein 1, beta | 79.75 | 80 |
| | | [HSPCB] | | |
| 167-65 | H-M16750 | PIM-1 proto-oncogene | 38 | 34.50 |
| | | serine/threonine-protein kinase | | |
| M311 A1 | H-M16827 | acyl-Coenzyme A dehydrogenase, | 46.42 | 50.0 kDa |
| | | C-4 to C-12 straight-chain. | | |
| D3 | H-M16961 | Alpha-2-HS-glycoprotein alpha | 40.48 | 50 |
| | | and beta chain | | |
| D3 | H-M16974 | Complement component 8, alpha | 64.35 | 55 |
| | | polypeptide | | |
| M248 C2 | H-M17017 | INTERLEUKIN-8 PRECURSOR | 11 | 11 |
| | | [Homo sapiens] | | |
| M305 E4 | H-M17885 | ribosomal phosphoprotein P0, | 34.98 | 37.0 kDa |
| | | acidic | | |
| M339 E2 | H-M17887 | ribosomal phosphoprotein P2 | 12.76 | 19.0 kDa |
| M248 D5 | H-M18731 | galactose-1-phosphate | 41.91 | 42 |
| | | uridylyltransferase [GALT] | | |
| F2 | H-M19309 | Troponin T1, skeletal, slow | 30.69 | 40 |
| M385 E2 | H-M19713 | tropomyosin, alpha, muscle | 31.35 | 41.0 kDa |
| 167-79 | H-M19722 | proto-oncogene tyrosine-protein | 64 | 58.26 |
| | | kinase FGR | | |
| M248 H1 | H-M20560 | Annexin III (lipocortin III), | 35.64 | 37 |
| | | INHIBITOR OF | | |
| | | PHOSPHOLIPASE A2 | | |
| M235 H1 | H-M20681 | GLUCOSE TRANSPORTER | 54.67 | 50 |
| | | TYPE 3, BRAIN | | |
| 167-29 | H-M21616 | beta platelet-derived growth | 121 | 121.7 |
| | | factor receptor precursor | | |
| M305 A3 | H-M21812 | myosin light chain 2 | 18.81 | 30 |
| 167-30 | H-M22146 | “40S ribosomal protein S4, x | 34 | 26.91 |
| | | isoform” | | |
| M302 D6 | H-M22430 | phospholipase A2 RASF-A | 15.95 | 31.0 kDa |
| E2 | H-M22491 | Bone morphogenetic protein 3 | 52.03 | 55 |
| | | (osteogenic) | | |
| M340 A2 | H-M22538 | NADH-ubiquinone reductase, 24 | 27.5 | 33 |
| | | kDa subunit, mitochondrial | | |
| B2 | H-M22632 | Glutamic-oxaloacetic | 47.41 | 47 |
| | | transaminase 2, mitochondrial | | |
| | | (aspartate-aminotransferase 2) | | |
| B4 | H-M22960 | Protective protein for beta- | 52.91 | 60 |
| | | galactosidase (galactosialidosis) | | |
| M250 C4 | H-M22995 | ras-related protein RAP1A, | | |
| | | member of RAS oncogene family | | |
| B3 | H-M23254 | Calpain, large polypeptide L2 | 77.11 | 77 |
| M266 B4 | H-M23613 | Nucleophosmin (nucleolar | 32.45 | 42 |
| | | phosphoprotein B23, numatrin), | | |
| | | BELIEVED TO BIND SINGLE- | | |
| | | STRANDED NUCLEIC ACIDS | | |
| M469 D2 | H-M23668 | Homo sapiens adrenodoxin gene | 20.35 | 25.0 kDa |
| M478 H3 | H-M24439 | Human liver/bone/kidney-type | 57.75 | 64.0 kDa |
| | | alkaline phosphatase (ALPL) | | |
| | | gene | | |
| F5 | H-M24470 | Glucose-6-phosphate | 38.06 | 44 |
| | | dehydrogenase | | |
| M270 E5 | H-M24898 | thyroid hormone triiodothyronine | 67.65 | 85 |
| | | receptor c-erbA, ear-1, Thyroid | | |
| | | hormone receptor, alpha (avian | | |
| | | erythroblastic leukemia viral (v- | | |
| | | erb-a) oncogene homolog) | | |
| D3 | H-M24902 | Acid phosphatase, prostate | 42.57 | 54 |
| D6 | H-M25809 | ATPase, H+ transporting, | 56.32 | 57 |
| | | lysosomal (vacuolar proton | | |
| | | pump), beta polypeptide, | | |
| | | 56/58 kD, isoform 1 | | |
| 167-77 | H-M26252 | “pyruvate kinase, M2 isozyme” | 60 | 58.48 |
| M271 F8 | H-M26326 | keratin 18 | 47.41 | 50.0 kDa |
| B1 | H-M26901 | Human renin gene | 44.44 | 50 |
| M271 G4 | H-M27396 | asparagine synthetase | 61.82 | 62 |
| M338 B3 | H-M27542 | globulin, sex hormone-binding | 39.200 | 40 |
| M512 B6 | H-M27602 | Protease, serine, 2 (trypsin 2) | 27.28 | 36.0 kDa |
| M270 B6 | H-M27691 | DNA-binding protein CREB, | 36.08 | 50 |
| | | cAMP-responsive | | |
| C1 | H-M27878 | Zinc finger protein 84 (HPF2) | 81.29 | 81 |
| M270 F6 | H-M28209 | guanine nucleotide-binding | 22.66 | 30.0 kDa |
| | | protein rab1 | | |
| M512 H5 | H-M28210 | RAB3A, member RAS oncogene | 24.31 | 36.0 kDa |
| | | family | | |
| B3 | H-M28214 | Homo sapiens GTP-binding | 24.2 | 34 |
| | | protein (RAB3B) mRNA, | | |
| | | complete cds | | |
| M300 C5 | H-M28249 | integrin, alpha 2 (CD49B, alpha 2 | 130.02 | 130.0 kDa |
| | | subunit of VLA-2 receptor) | | |
| | | [ITGA2] | | |
| M248 B6 | H-M28372 | zinc finger protein 9 (a cellular | 19.58 | 28.0 kDa |
| | | retroviral nucleic acid binding | | |
| | | protein) [ZNF9] | | |
| M248 C5 | H-M28983 | interleukin 1, alpha [IL1A] | 29.92 | 42 |
| M298 C1 | H-M29536 | translation initiation factor 2, beta | 36.74 | 50.0 kDa |
| | | subunit | | |
| M425 A5 | H-M29696 | Interleukin 7 receptor | 50.6 | 63.0 kDa |
| E1 | H-M29960 | Human steroid receptor (TR2-11) | 66.44 | 65 |
| | | mRNA, complete cds | | |
| M361 D3 | H-M29971 | 6-O-methylguanine-DNA | 22.88 | 33.0 kDa |
| | | methyltransferase [MGMT] | | |
| 167-67 | H-M30448 | “casein kinase II, beta chain” | 34 | 23.72 |
| M250 E2 | H-M31211 | MYOSIN LIGHT CHAIN 1, | 22.99 | 30.0 kDa |
| | | SLOW-TWITCH MUSCLE A | | |
| | | ISOFORM [Homo sapiens] | | |
| M311 C4 | H-M31452 | proline-rich protein | 65.78 | 68 |
| M312 H3 | H-M31469 | ras-like protein TC4 | 23.87 | 32.0 kDa |
| 167-41 | H-M31606 | “phosphorylase B kinase gamma | 50 | 44.7 |
| | | catalytic chain, testis isoform” | | |
| B4 | H-M31642 | Hypoxanthine | 24.09 | 36 |
| | | phosphoribosyltransferase 1 | | |
| | | (Lesch-Nyhan syndrome) | | |
| M416 D8 | H-M31932 | Fc fragment of IgG, low affinity | 34.98 | 45.0 kDa |
| | | IIa, receptor for (CD32) | | |
| M305 A8 | H-M32011 | neutrophil cytosolic factor 2 | 57.97 | 58 |
| | | (65 kD, chronic granulomatous | | |
| | | disease, autosomal 2) [NCF2] | | |
| B2 | H-M32315 | Human tumor necrosis factor | 50.82 | 60 |
| | | receptor mRNA, complete cds | | |
| M266 C2 | H-M33374 | cell adhesion protein SQM1 | 14.96 | 18.0 kDa |
| M431 F1 | H-M33375 | dihydrodiol dehydrogenase 4 | 33.99 | 40.0 kDa |
| G6 | H-M33680 | Human 26-kDa cell surface | 26.07 | 24 |
| | | protein TAPA-1 mRNA, complete | | |
| | | cds | | |
| F1 | H-M33772 | Human fast skeletal muscle | 17.71 | 29 |
| | | troponin C gene | | |
| 167-15 | H-M34065 | m-phase inducer phosphatase 3 | 55 | 52.10 |
| F4 | H-M34079 | Human immunodeficiency virus | 44.55 | 52 |
| | | tat transactivator binding protein- | | |
| | | 1 (tbp-1) mRNA, complete cds | | |
| 169-86 | H-M34181 | “cAMP-dependent protein kinase, | 50 | 38.68 |
| | | beta-catalytic subunit” | | |
| D1 | H-M34379 | Elastatse 2, neutrophil | 29.48 | 35 |
| M314 E1 | H-M34671 | CD59 glycoprotein precursor | 14.150 | 20 |
| M266 C3 | H-M35252 | CO-029 (GB: M35252) | 26.18 | 30 |
| M315 A4 | H-M36035 | benzodiazapine receptor | 18.7 | 19 |
| | | (peripheral) [BZRP] | | |
| M300 C1 | H-M36340 | ADP-ribosylation factor 1 | 20.02 | 30 |
| M312 C3 | H-M36341 | ADP-ribosylation factor 2 | 19.91 | 29 |
| D6 | H-M36634 | Vasoactive intestinal peptide | 18.81 | 28 |
| 169-26 | H-M36881 | proto-oncogene tyrosine-protein | 60 | 56.06 |
| | | kinase LCK | | |
| 167-76 | H-M36981 | nucleoside diphosphate kinase B | 26 | 16.79 |
| M298 D6 | H-M37400 | aspartate aminotransferase, | 45.54 | 50.0 kDa |
| | | cytosolic | | |
| 167-88 | H-M37712 | galactosyltransferase associated | 55 | 48.36 |
| | | protein kinase P58/GTA | | |
| M424 F4 | H-M38258 | Retinoic acid receptor, gamma 1 | 50.05 | 58.0 kDa |
| M266 H3 | H-M38690 | CD9 antigen, INVOLVED IN | 25.19 | 26.0 kDa |
| | | PLATELET ACTIVATION AND | | |
| | | AGGREGATION. | | |
| M270 A5 | H-M55265 | casein kinase II, alpha catalytic | 43.12 | 50 |
| | | subunit | | |
| 169-74 | H-M55284 | human protein kinase C-L | 80 | 75.09 |
| | | (PRKCL) mRNA | | |
| M512 B3 | H-M55514 | Potassium voltage-gated channel, | 71.94 | 100.0 kDa |
| | | shaker-related subfamily, | | |
| | | member 4 | | |
| M271 F5 | H-M57567 | ADP-ribosylation factor 5 [AR5]. | 19.91 | 32.0 kDa |
| | | INVOLVED IN PROTEIN | | |
| | | TRAFFICKING AND ACTS AS | | |
| | | AN ALLOSTERIC ACTIVATOR | | |
| | | OF CHOLERA TOXIN. | | |
| M250 D1 | H-M57627 | interleukin 10 [IL10], | 19.69 | 27 |
| | | SUPPRESSOR FACTOR FOR | | |
| | | THI IMMUNE RESPONSES | | |
| | | (BY SIMILARITY). | | |
| M302 D3 | H-M57730 | EPH-related receptor tyrosine | 22.620 | 36.0 kDa |
| | | kinase ligand 1 precursor | | |
| M248 B5 | H-M58458 | ribosomal protein S4, X-linked | 29.04 | 36.0 kDa |
| | | [RPS4X] | | |
| M248 A5 | H-M58459 | ribosomal protein S4, Y-linked | 29.04 | 36 |
| | | [RPS4Y] | | |
| M248 G5 | H-M58525 | CATECHOL O- | 29.92 | 36 |
| | | METHYLTRANSFERASE, | | |
| | | MEMBRANE-BOUND FORM | | |
| | | [Homo sapiens], COMT | | |
| M482 B2 | H-M59916 | Sphingomyelin phosphodiesterase | 69.3 | 69.0 kDa |
| | | 1, acid lysosomal (acid | | |
| | | sphingomyelinase) | | |
| M390 C1 | H-M60091 | galactose-1-phosphate | 41.8 | 50.0 kDa |
| | | uridylyltransferase | | |
| M316 B1 | H-M60314 | bone morphogenetic protein 5 | 50.05 | 55 |
| | | [BMP5] | | |
| B4 | H-M60459 | Erythropoietin receptor | 55.99 | 60 |
| C7 | H-M60483 | Human protein phosphatase 2A | 34.1 | 56 |
| | | catalytic subunit-alpha gene, | | |
| | | complete cds | | |
| M462 D7 | H-M60484 | Human protein phosphatase 2A | 34.1 | 44.0 kDa |
| | | catalytic subunit-beta gene, | | |
| | | complete cds | | |
| A12 | H-M60527 | deoxycytidine kinase | 28.670 | 50 |
| 167-5 | H-M60724 | human p70 ribosomal S6 kinase | 66 | 57.82 |
| | | alpha-I mRNA | | |
| 167-17 | H-M60725 | human p70 ribosomal S6 kinase | 62 | 55.29 |
| | | alpha-II mRNA | | |
| M271 A4 | H-M61199 | cleavage signal 1, ESTs, Highly | 27.5 | 36.0 kDa |
| | | similar to CLEAVAGE SIGNAL- | | |
| | | 1 PROTEIN [Homo sapiens] | | |
| B1 | H-M61733 | Homo sapiens erythroid | 70.62 | 71 |
| | | membrane protein 4.1 mRNA, | | |
| | | complete cds | | |
| M298 A1 | H-M61764 | tubulin, gamma | 49.72 | 55.0 kDa |
| M422 E2 | H-M62505 | Complement component 5 | 38.61 | 38.0 kDa |
| | | receptor 1 (C5a ligand) | | |
| M313 G5 | H-M62810 | transcription factor 1, | 27.17 | 35.0 kDa |
| | | mitochondrial | | |
| C9 | H-M62839 | apolipoprotein H | 38.06 | 60 |
| G5 | H-M63154 | Gastric intrinsic factor (vitamin B | 45.98 | 52 |
| | | synthesis) | | |
| 167-6 | H-M63167 | RAC-alpha serine/threonine | 64 | 52.87 |
| | | kinase | | |
| B1 | H-M63573 | Peptidylprolyl isomerase B | 23.87 | 33 |
| | | (cyclophilin B) | | |
| M302 H2 | H-M63603 | phospholamban | 5.83 | 6 |
| M306 D1 | H-M63838 | interferon, gamma-inducible | 80.3 | 108 |
| | | protein 16 | | |
| M423 H3 | H-M63959 | Low density lipoprotein-related | 39.38 | 48.0 kDa |
| | | protein-associated protein 1 | | |
| | | (alpha-2-macroglobulin receptor- | | |
| | | associated protein 1 | | |
| G3 | H-M64099 | Human gamma-glutmyl | 64.57 | 52 |
| | | transpeptidase-related protein | | |
| | | (GGT-Rel) mRNA, complete cds | | |
| M475 B8 | H-M64673 | Human heat shock factor 1 | 58.3 | 65.0 kDa |
| | | (TCF5) mRNA, complete cds | | |
| M266 D5 | H-M64716 | ribosomal protein S25 | 13.86 | 17.0 kDa |
| M248 C6 | H-M64752 | glutamate receptor, ionotropic, | 99.88 | 100 |
| | | AMPA 1 [GRIAI] | | |
| M312 G3 | H-M64925 | palmitoylated membrane protein, | 51.37 | 51.0 kDa |
| | | erythrocyte, 55 kDa | | |
| M302 C7 | H-M65292 | complement factor H-related | 36.41 | 50 |
| | | protein (GB: M65292) | | |
| D3 | H-M68516 | Human protein C inhibitor gene, | 44.77 | 54 |
| | | complete cds | | |
| 167-27 | H-M68520 | cell division protein kinase 2 | 38 | 32.85 |
| M236 D5 | H-M68867 | Cellular retinoic acid-binding | 15.29 | 19.0 kDa |
| | | protein 2, MAY REGULATE | | |
| | | THE ACCESS OF RETINOIC | | |
| | | ACID TO THE NUCLEAR | | |
| | | RETINOIC ACID RECEPTORS. | | |
| M441 E1 | H-M69226 | monoamine oxidase A [MAOA] | 58.08 | 64.0 kDa |
| M298 D5 | H-M72393 | calcium-dependent phospholipid- | 82.5 | 117.0 kDa |
| | | binding protein [PLA2*] | | |
| M422 D5 | H-M73238 | Ciliary neurotrophic factor | 41.03 | 51.0 kDa |
| | | receptor | | |
| C1 | H-M73255 | Human vascular cell adhesion | 81.4 | 81 |
| | | molecule-1 (VCAM1) gene, | | |
| | | complete CDS | | |
| M422 G6 | H-M73481 | Human gastrin releasing peptide | 42.35 | 45.0 kDa |
| | | receptor (GRPR) mRNA, | | |
| | | complete cds | | |
| M235 G6 | H-M73499 | carboxylesterase, INVOLVED IN | 62.48 | 90.0 kDa |
| | | THE DETOXIFICATION OF | | |
| | | XENOBIOTICS AND THE | | |
| | | ACTIVATION OF ESTER AND | | |
| | | AMIDE PRODRUGS. | | |
| M302 D1 | H-M73547 | polyposis locus DP1 | 20.46 | 28 |
| M300 H4 | H-M73969 | interleukin 8 receptor, beta | 39.71 | 36 |
| | | [IL8RB] | | |
| G1 | H-M74491 | ADP-ribosylation factor 3 | 20.02 | 31 |
| B4 | H-M74816 | | 49.5 | 50 |
| B2 | H-M75110 | H, K-ATPase, beta subunit | 32.12 | 37 |
| M416 B8 | H-M76766 | General transcription factor IIB | 34.87 | 44.0 kDa |
| 167-18 | H-M77198 | RAC-beta serine/threonine kinase | 64 | 57.27 |
| 167-87 | H-M77348 | PMEL 17 protein precursor | 74 | 73.55 |
| C4 | H-M77698 | YY1 transcription factor | 45.65 | 48 |
| M248 G6 | H-M80261 | apurinic/apyrimidinic (abasic) | 35.09 | 37.0 kDa |
| | | endonuclease [APE], REPAIRS | | |
| | | OXIDATIVE DNA DAMAGES | | |
| | | IN VITRO | | |
| 169-50 | H-M80359 | putative serine/threonine-protein | 80 | 78.50 |
| | | kinase P78 | | |
| M330 H1 | H-M80461 | immunoglobulin-associated beta | 25.370 | 27.0 kDa |
| | | (B29) [IGB] | | |
| 169-1 | H-M80613 | ring3 protein | 100 | 83.01 |
| M298 A2 | H-M80783 | B12 protein | 34.87 | 43.0 kDa |
| 217-1 | H-M81457 | calpactin 1 light chain | 10 | 10.74 |
| M422 C6 | H-M81589 | Homo sapiens serotonin 1D | 41.58 | 41.0 kDa |
| | | receptor (5-HTID) mRNA, | | |
| | | complete cds | | |
| M424 A1 | H-M81590 | Homo sapiens serotonin 1D | 43.01 | 48.0 kDa |
| | | receptor (5-HTID-) mRNA, | | |
| | | complete cds | | |
| M250 H1 | H-M81592 | gamma-glutamyl carboxylase | 83.49 | 85 |
| | | [GGCX], CONVERTS | | |
| | | GLUTAMATE RESIDUES TO | | |
| | | GAMMA- | | |
| | | CARBOXYGLUTAMATE | | |
| M250 F2 | H-M81601 | TRANSCRIPTION | 33.22 | 36.0 kDa |
| | | ELONGATION FACTOR S-II | | |
| | | [Homo sapiens] | | |
| C2 | H-M81650 | Human semenogelin 1 (SEMGI) | 50.93 | 52 |
| | | gene, complete cds | | |
| M266 A4 | H-M81757 | ribosomal protein S19 | 16.06 | 18 |
| 169-61 | H-M81933 | m-phase inducer phosphatase 1 | 57 | 57.60 |
| M302 H1 | H-M82809 | annexin IV | 35.42 | 38.0 kDa |
| M300 C4 | H-M83653 | cytoplasmic phosphotyrosyl | 17.49 | 28.0 kDa |
| | | protein phosphatase, type I | | |
| 169-14 | H-M83941 | tyrosne-protein kinase receptor | 108 | 108.2 |
| | | ETK1 precursor | | |
| F1 | H-M84443 | Galactokinase 2 | 50.49 | 52 |
| M305 H6 | H-M84747 | interleukin 9 receptor [IL9R] | 57.53 | 58 |
| 167-53 | H-M86400 | 14-3-3 protein zeta/delta | 33 | 27.02 |
| M271 C8 | H-M86521 | transketolase | 68.64 | 68.0 kDa |
| 169-51 | H-M86699 | human kinase (TTK) mRNA | 92 | 92.58 |
| M316 F2 | H-M86752 | transformation-sensitive protein | 59.84 | 60.0 kDa |
| M270 C8 | H-M86921 | membrane glycoprotein mb-1, | 24.97 | 34 |
| | | Immunoglobulin-associated | | |
| | | alpha, ASSOCIATED TO | | |
| | | SURFACE IGM-RECEPTOR; | | |
| | | MAY BE INVOLVED IN | | |
| | | SIGNAL TRANSDUCTION | | |
| A5 | H-M87507 | Homo sapien interleukin-1 beta | 44.55 | 50 |
| | | convertase (IL1BCE) mRNA, | | |
| | | complete cds | | |
| M305 B7 | H-M88011 | glucokinase [GCK] | 51.26 | 60 |
| M305 H1 | H-M88279 | immunophilin FKBP52 | 50.6 | 64.0 kDa |
| M420 F1 | H-M88468 | mevalonate kinase | 43.600 | 47.0 kDa |
| M305 A7 | H-M89913 | dUTP pyrophosphatase | 15.62 | 19 |
| | | (dUTPase) [DUT*] | | |
| M316 E2 | H-M90657 | tumor-associated antigen L6 | 22.33 | 28 |
| 167-31 | H-M90813 | human D-type cyclin (CCND2) | 36 | 31.86 |
| | | mRNA | | |
| A1 | H-M91036 | H. sapiens G-gamma globin and | 16.28 | 18 |
| | | A-gamma globin genes, complete | | |
| | | cds's | | |
| G2 | H-M91463 | Human glucose transporter | 55.66 | 52 |
| | | (GLUT4) gene, complete cds | | |
| A1 | H-M91670 | Human ubiquitin carrier protein | 24.86 | 36 |
| | | (E2-EPF) mRNA, complete cds | | |
| E4 | H-M92444 | Homo sapiens | 35.09 | 45 |
| | | apurinic/apyrimidinic | | |
| | | endonuclease (HAP1) gene, | | |
| | | complete cds | | |
| M305 C4 | H-M94556 | single-stranded DNA-binding | 16.39 | 20 |
| | | protein, mitochondrial | | |
| G12 | H-M94856 | fatty-acid-binding protein | 14.96 | 36 |
| | | homolog | | |
| M453 C3 | H-M95623 | Homo sapiens | 39.82 | 50.0 kDa |
| | | hydroxymethylbilane synthase | | |
| | | gene, complete cds | | |
| M302 F2 | H-M95787 | smooth muscle protein SM22 | 22.22 | 33.0 kDa |
| A1 | H-M95809 | Human basic transcription factor | 60.39 | 64 |
| | | 62 kD subunit (BTF2), complete | | |
| | | cds | | |
| M271 E8 | H-M96982 | small nuclear ribonucleoprotein | 26.51 | 39.0 kDa |
| | | U2 auxiliary factor, 35 kDa, | | |
| | | SPLICING FACTOR U2AF 35 | | |
| | | KD SUBUNIT. NECESSARY | | |
| | | FOR THE SPLICING OF PRE- | | |
| | | mRNA. | | |
| M416 B3 | H-M96995 | Growth factor receptor-bound | 23.98 | 32.0 kDa |
| | | protein 2 | | |
| G2 | H-M96995 | Growth factor receptor-bound | 23.98 | 49 |
| | | protein 2 | | |
| H4 | H-M97016 | Bone morphogenetic protein 8 | 44.33 | 61 |
| | | (osteogenic protein 2) | | |
| M271 D1 | H-M97190 | Sp2 transcription factor [SP2], | 54.56 | 60 |
| | | BINDS TO GC BOX | | |
| | | PROMOTERS ELEMENTS AND | | |
| | | SELECTIVELY ACTIVATES | | |
| | | mRNA SYNTHESIS FROM | | |
| | | GENES THAT CONTAIN | | |
| | | FUNCTIONAL RECOGNITION | | |
| | | SITES. | | |
| M271 C1 | H-M97191 | Sp3 transcription factor [SP3], | 71.94 | 72 |
| | | BINDS TO GT AND GC BOXES | | |
| | | PROMOTERS ELEMENTS. | | |
| | | PROBABLE | | |
| | | TRANSCTRIPTIONAL | | |
| | | ACTIVATOR. | | |
| M305 C7 | H-M97388 | transcription repressor (interacting | 19.47 | 30 |
| | | with the TATA-binding protein) | | |
| | | [DR1*] | | |
| 217-13 | H-M97675 | human transmembrane receptor | 100 | 103.1 |
| | | (ror1) mRNA | | |
| B3 | H-M97856 | Nuclear autoantigenic sperm | 86.68 | 87 |
| | | protein (histone-binding) | | |
| M429 G2 | H-M97935 | Homo sapiens transcription factor | 82.61 | 89.0 kDa |
| | | ISGF-3 mRNA, complete cds | | |
| D1 | H-M99487 | Human prostate-specific | 82.61 | 92 |
| | | membrane antigen (PSM) mRNA, | | |
| | | complete cds | | |
| M363 A1 | H-P0002 | riboflavin synthase beta chain | 17.27 | |
| | | (ribE) | | |
| M363 B1 | H-P0004 | carbonic anhydrase (icfA) | 24.42 | |
| M363 C1 | H-P0005 | orotidine 5′-phosphate | 25.08 | |
| | | decarboxylase (pyrF) | | |
| M363 D1 | H-P0006 | pantoate-beta-alanine ligase | 30.47 | |
| | | (panC) | | |
| M379 A1 | H-P0010-2 | chaperone and heat shock protein | 60.17 | |
| | | (groEL) | | |
| M363 E1 | H-P0011 | co-chaperone (groES) | 13.09 | |
| M363 F1 | H-P0012 | DNA primase (dnaG) | 61.6 | |
| M363 G1 | H-P0013 | hypothetical protein | 38.61 | |
| M363 H1 | H-P0014 | hypothetical protein | 30.36 | |
| M363 A2 | H-P0015 | hypothetical protein | 10.34 | |
| M363 B2 | H-P0016 | hypothetical protein | 9.68 | |
| M363 C2 | H-P0017 | virB4 homolog (virB4) | 86.68 | |
| M363 D2 | H-P0018 | hypothetical protein | 51.7 | |
| M363 E2 | H-P0021 | hypothetical protein | 21.01 | |
| M363 F2 | H-P0022 | conserved hypothetical integral | 57.42 | |
| | | membrane protein | | |
| M363 G2 | H-P0026 | citrate synthase (gltA) | 46.97 | |
| M363 H2 | H-P0027 | isocitrate dehydrogenase (icd) | 46.86 | |
| M363 A3 | H-P0028 | conserved hypothetical secreted | 19.58 | |
| | | protein | | |
| M363 B3 | H-P0030 | hypothetical protein | 65.34 | |
| M363 C3 | H-P0031 | hypothetical protein | 15.18 | |
| M363 D3 | H-P0034 | aspartate 1-decarboxylase (panD) | 12.98 | |
| M363 E3 | H-P0035 | conserved hypothetical protein | 10.78 | |
| M363 F3 | H-P0037 | NADH-ubiquinone | 38.72 | |
| | | oxidoreductase subunit | | |
| M363 G3 | H-P0044 | GDP-D-mannose dehydratase | 42.02 | |
| | | (rfbD) | | |
| M363 H3 | H-P0047 | hydrogenase expression/formation | 36.63 | |
| | | protein (hypE) | | |
| M363 A4 | H-P0048 | transcriptional regulator (hypF) | 84.7 | |
| M363 B4 | H-P0052 | hypothetical protein | 36.41 | |
| M363 C4 | H-P0055 | proline permease (putP) | 54.67 | |
| M363 D4 | H-P0056 | delta-1-pyrroline-5-carboxylate | 130.46 | |
| | | dehydrogenase | | |
| M363 E4 | H-P0057 | hypothetical protein | 7.7 | |
| M363 F4 | H-P0063 | hypothetical protein | 54.67 | |
| M363 G4 | H-P0064 | hypothetical protein | 15.4 | |
| M363 H4 | H-P0066 | conserved hypothetical ATP- | 91.52 | |
| | | binding protein | | |
| M363 A5 | H-P0067 | urease accessory protein (ureH) | 29.26 | |
| M363 B5 | H-P0068 | urease accessory protein (ureG) | 22 | |
| M363 C5 | H-P0075 | urease protein (ureC) | 49.06 | |
| M363 D5 | H-P0077 | peptide chain release factor RF-1 | 38.83 | |
| | | (prfA) | | |
| M363 E5 | H-P0082 | methyl-accepting chemotaxis | 74.14 | |
| | | transducer (tlpC) | | |
| M363 F5 | H-P0086 | conserved hypothetical protein | 49.61 | |
| M363 G5 | H-P0087 | hypothetical protein | 50.38 | |
| M363 H5 | H-P0088 | RNA polymerase sigma-70 factor | 73.92 | |
| | | (rpoD) | | |
| M363 A6 | H-P0089 | pfs protein (pfs) | 25.52 | |
| M363 B6 | H-P0090 | malonyl coenzyme A-acyl carrier | 34.1 | |
| | | protein transacylase (fabD) | | |
| M363 C6 | H-P0093 | hypothetical protein | 12.21 | |
| M363 D6 | H-P0096 | phosphoglycerate dehydrogenase | 34.65 | |
| M304 A1 | H-P0099 | methyl-accepting chemotaxis | 74.36 | |
| | | protein (tlpA) | | |
| M304 B1 | H-P0100 | conserved hypothetical protein | 40.59 | |
| M304 C1 | H-P0101 | hypothetical protein | 27.94 | |
| M304 D1 | H-P0104 | 2',3'-cyclic-nucleotide 2'- | 64.02 | |
| | | phosphodiesterase (cpdB) | | |
| M304 E1 | H-P0105 | conserved hypothetical protein | 17.16 | |
| M304 F1 | H-P0106 | cystathionine gamma-synthase | 41.91 | |
| | | (metB) | | |
| M304 G1 | H-P0107 | cysteine synthetase (cysK) | 33.77 | |
| M304 H1 | H-P0108 | hypothetical protein | 20.57 | |
| M304 A2 | H-P0109 | chaperone and heat shock protein | 68.31 | |
| | | 70 (dnaK) | | |
| M304 B2 | H-P0110 | co-chaperone and heat shock | 20.9 | |
| | | protein (grpE) | | |
| M304 C2 | H-P0111 | hypothetical protein | 30.47 | |
| M304 D2 | H-P0113 | hypothetical protein | 10.89 | |
| M304 E2 | H-P0114 | hypothetical protein | 69.19 | |
| M304 F2 | H-P0115 | flagellin B (flaB) | 56.65 | |
| M304 G2 | H-P0116 | DNA topoisomerase I (topA) | 81.07 | |
| M304 H2 | H-P0117 | conserved hypothetical protein | 33.99 | |
| M304 A3 | H-P0118 | hypothetical protein | 43.56 | |
| M304 B3 | H-P0119 | hypothetical protein | 50.82 | |
| M304 C3 | H-P0120 | hypothetical protein | 43.89 | |
| M304 D3 | H-P0121 | phosphoenolpyruvate synthase | 89.43 | |
| | | (ppsA) | | |
| M304 E3 | H-P0122 | hypothetical protein | 4.84 | |
| M304 F3 | H-P0123 | threonyl-tRNA synthetase (thrS) | 67.43 | |
| M304 G3 | H-P0124 | translation initiation factor IF-3 | 22.44 | |
| | | (infC) | | |
| M304 H3 | H-P0125 | ribosomal protein L35 (rpl35) | 7.15 | |
| M304 A4 | H-P0126 | ribosomal protein L20 (rpl20) | 12.87 | |
| M304 B4 | H-P0127 | outer membrane protein (omp4) | 31.57 | |
| M304 C4 | H-P0128 | hypothetical protein | 4.62 | |
| M304 D4 | H-P0129 | hypothetical protein | 15.62 | |
| M304 E4 | H-P0130 | hypothetical protein | 31.57 | |
| M304 F4 | H-P0131 | hypothetical protein | 3.74 | |
| M304 G4 | H-P0132 | L-serine deaminase (sdaA) | 50.16 | |
| M304 H4 | H-P0133 | serine transporter (sdaC) | 45.54 | |
| M304 A5 | H-P0134 | 3-deoxy-D-arabino-heptulosonate | 49.5 | |
| | | 7-phosphate synthase (dhsI) | | |
| M304 B5 | H-P0135 | hypothetical protein | 4.95 | |
| M304 C5 | H-P0136 | bacterioferritin comigratory | 16.83 | |
| | | protein (bcp) | | |
| M304 D5 | H-P0137 | hypothetical protein | 23.32 | |
| M304 E5 | H-P0138 | conserved hypothetical iron-sulfur | 53.02 | |
| | | protein | | |
| M304 F5 | H-P0139 | conserved hypothetical secreted | 26.73 | |
| | | protein | | |
| M304 G5 | H-P0140 | L-lactate permease (lctP) | 60.5 | |
| M304 H5 | H-P0141 | L-lactate permease (lctP) | 60.72 | |
| M304 A6 | H-P0142 | A/G-specific adenine glycosylase | 36.19 | |
| | | (mutY) | | |
| M304 B6 | H-P0144 | cytochrome c oxidase, heme b | 53.79 | |
| | | and copper-binding subunit, | | |
| | | membrane-bound (fixN) | | |
| M304 C6 | H-P0145 | cytochrome c oxidase, monoheme | 25.63 | |
| | | subunit, membrane-bound (fixO) | | |
| M304 D6 | H-P0146 | cbb3-type cytochrome c oxidase | 8.14 | |
| | | subunit Q (CcoQ) | | |
| M304 E6 | H-P0147 | cytochrome c oxidase, diheme | 31.57 | |
| | | subunit, membrane-bound (fixP) | | |
| M304 F6 | H-P0148 | hypothetical protein | 7.59 | |
| M304 G6 | H-P0150 | hypothetical protein | 21.67 | |
| M304 H6 | H-P0152 | hypothetical protein | 31.68 | |
| M304 A7 | H-P0153 | recombinase (recA) | 38.28 | |
| M304 B7 | H-P0154 | enolase (eno) | 46.97 | |
| M304 C7 | H-P0155 | hypothetical protein | 10.12 | |
| M304 D7 | H-P0157 | shikimic acid kinase 1 (aroK) | 17.93 | |
| M304 E7 | H-P0158 | hypothetical protein | 35.09 | |
| M304 F7 | H-P0159 | lipopolysaccharide 1,2- | 41.03 | |
| | | glucosyltransferase (rfaJ) | | |
| M304 G7 | H-P0161 | hypothetical protein | 4.07 | |
| M304 H7 | H-P0162 | conserved hypothetical protein | 26.51 | |
| M304 A8 | H-P0163 | delta-aminolevulinic acid | 35.64 | |
| | | dehydratase (hemB) | | |
| M304 B8 | H-P0164 | signal-transducing protein, | 28.05 | |
| | | histidine kinase | | |
| M304 C8 | H-P0165 | hypothetical protein | 19.14 | |
| M304 D8 | H-P0166 | response regulator (ompR) | 24.86 | |
| M304 E8 | H-P0167 | hypothetical protein | 17.38 | |
| M304 F8 | H-P0168 | hypothetical protein | 9.68 | |
| M304 G8 | H-P0170 | hypothetical protein | 27.94 | |
| M304 H8 | H-P0171 | peptide chain release factor RF-2 | 40.04 | |
| | | (prfB) | | |
| M304 A9 | H-P0172 | molybdopterin biosynthesis | 43.12 | |
| | | protein (moeA) | | |
| M304 B9 | H-P0173 | flagellar biosynthetic protein | 28.16 | |
| | | (fliR) | | |
| M304 C9 | H-P0174 | hypothetical protein | 28.49 | |
| M304 D9 | H-P0175 | cell binding factor 2 | 33 | |
| M304 E9 | H-P0176 | fructose-bisphosphate aldolase | 33.88 | |
| | | (tsr) | | |
| M304 F9 | H-P0177 | translation elongation factor EF-P | 20.68 | |
| | | (efp) | | |
| M304 G9 | H-P0178 | spore coat polysaccharide | 37.51 | |
| | | biosynthesis protein E | | |
| M304 H9 | H-P0179 | ABC transporter, ATP-binding | 23.54 | |
| | | protein | | |
| M304 A10 | H-P0180 | apolipoprotein N-acyltransferase | 46.86 | |
| | | (cute) | | |
| M304 B10 | H-P0182 | lysyl-tRNA synthetase (lysS) | 55.22 | |
| M304 C10 | H-P0183 | serine hydroxymethyltransferase | 45.87 | |
| | | (glyA) | | |
| M304 D10 | H-P0184 | hypothetical protein | 19.91 | |
| M304 E10 | H-P0185 | hypothetical protein | 29.48 | |
| M304 F10 | H-P0186 | hypothetical protein | 44.55 | |
| M304 G10 | H-P0187 | hypothetical protein | 10.56 | |
| M304 H10 | H-P0188 | hypothetical protein | 3.74 | |
| M304 A11 | H-P0189 | conserved hypothetical integral | 19.58 | |
| | | membrane protein | | |
| M304 B11 | H-P0190 | conserved hypothetical secreted | 55.33 | |
| | | protein | | |
| M304 C11 | H-P0191 | fumarate reductase, iron-sulfur | 27.06 | |
| | | subunit (frdB) | | |
| M304 D11 | H-P0192 | fumarate reductase, flavoprotein | 78.65 | |
| | | subunit (frdA) | | |
| M304 E11 | H-P0193 | fumarate reductase, cytochrome b | 28.16 | |
| | | subunit (frdC) | | |
| M304 F11 | H-P0194 | triosephosphate isomerase (tpi) | 25.85 | |
| M304 G11 | H-P0195 | enoyl-(acyl-carrier-protein) | 30.36 | |
| | | reductase (NADH) (fabI) | | |
| M365 A1 | H-P0197 | S-adenosylmethionine synthetase | 42.46 | |
| | | 2 (metX) | | |
| M365 B1 | H-P0203 | hypothetical protein | 10.12 | |
| M365 C1 | H-P0209 | hypothetical protein | 49.61 | |
| M365 D1 | H-P0213 | glucose inhibited division protein | 68.42 | |
| | | (gidA) | | |
| M381 E1 | H-P0218 | hypothetical protein | 20.24 | |
| M365 E1 | H-P0221 | nifU-like protein | 35.97 | |
| M365 F1 | H-P0227 | outer membrane protein (omp5) | 76.12 | |
| M365 G1 | H-P0228 | conserved hypothetical integral | 43.01 | |
| | | membrane protein | | |
| M365 H1 | H-P0230 | CTP: CMP-3-deoxy-D-manno- | 26.84 | |
| | | octulosonate-cytidylyl-transferase | | |
| | | (kdsB) | | |
| M365 A2 | H-P0233 | conserved hypothetical protein | 43.01 | |
| M365 B2 | H-P0235 | conserved hypothetical secreted | 39.16 | |
| | | protein | | |
| M365 C2 | H-P0236 | hypothetical protein | 13.64 | |
| M365 D2 | H-P0238 | prolyl-tRNA synthetase (proS) | 63.58 | |
| M381 E2 | H-P0243 | neutrophil activating protein | 15.95 | |
| | | (napA) (bacterioferritin) | | |
| M365 E2 | H-P0244 | signal-transducing protein, | 42.02 | |
| | | histidine kinase (atoS) | | |
| M365 F2 | H-P0246 | flagellar basal-body P-ring protein | 37.73 | |
| | | (flgI) | | |
| M365 G2 | H-P0247 | ATP-dependent RNA helicase, | 54.23 | |
| | | DEAD-box family (deaD) | | |
| M365 H2 | H-P0248 | conserved hypothetical protein | 39.93 | |
| M379 B1 | H-P0249-2 | hypothetical protein | 19.8 | |
| M379 C1 | H-P0250-2 | oligopeptide ABC transporter, | 56.87 | |
| | | ATP-binding protein (oppD) | | |
| M381 A3 | H-P0251 | oligopeptide ABC transporter, | 37.29 | |
| | | permease protein (oppC) | | |
| M379 E1 | H-P0252-2 | outer membrane protein (omp7) | 53.68 | |
| M365 A3 | H-P0254 | outer membrane protein (omp8) | 47.52 | |
| M365 B3 | H-P0255 | adenylosuccinate synthetase | 45.32 | |
| | | (purA) | | |
| M365 C3 | H-P0257 | conserved hypothetical secreted | 24.2 | |
| | | protein | | |
| M365 D3 | H-P0259 | exonuclease VII, large subunit | 46.31 | |
| | | (xseA) | | |
| M381 D3 | H-P0260 | adenine specific DNA | 42.35 | |
| | | methyltransferase (mod) | | |
| M365 E3 | H-P0263 | adenine specific DNA | 27.83 | |
| | | methyltransferase (hpaim) | | |
| M365 F3 | H-P0264 | ATP-dependent protease binding | 94.27 | |
| | | subunit (clpB) | | |
| M365 G3 | H-P0266 | dihydroorotase (pyrC) | 41.69 | |
| M365 H3 | H-P0267 | chlorohydrolase | 45.1 | |
| M365 A4 | H-P0271 | hypothetical protein | 36.08 | |
| M365 B4 | H-P0275 | ATP-dependent nuclease (addB) | 47.41 | |
| M381 G3 | H-P0276 | hypothetical protein | 20.46 | |
| M365 C4 | H-P0278 | guanosine pentaphosphate | 53.35 | |
| | | phosphohydrolase (gppA) | | |
| M365 D4 | H-P0279 | lipopolysaccharide | 37.51 | |
| | | heptosyltransferase-1 (rfaC) | | |
| M365 E4 | H-P0280 | heat shock protein B (ibpB) | 36.19 | |
| M365 F4 | H-P0282 | hypothetical protein | 52.91 | |
| M365 G4 | H-P0283 | 3-dehydroquinate synthase (aroB) | 37.84 | |
| M365 H4 | H-P0284 | conserved hypothetical integral | 57.64 | |
| | | membrane protein | | |
| M365 A5 | H-P0285 | conserved hypothetical protein | 46.09 | |
| M381 A4 | H-P0287 | hypothetical protein | 19.03 | |
| M381 C4 | H-P0288 | hypothetical protein | 17.38 | |
| M366 A1 | H-P0389 | superoxide dismutase (sodB) | 23.54 | |
| M366 B1 | H-P0390 | adhesin-thiol peroxidase (tagD) | 18.37 | |
| M366 C1 | H-P0391 | purine-binding chemotaxis | 18.26 | |
| | | protein (cheW) | | |
| M366 D1 | H-P0392 | histidine kinase (cheA) | 88.44 | |
| M366 E1 | H-P0393 | chemotaxis protein (cheV) | 34.32 | |
| M366 F1 | H-P0394 | hypothetical protein | 27.83 | |
| M366 G1 | H-P0395 | conserved hypothetical protein | 24.53 | |
| M366 H1 | H-P0396 | conserved hypothetical protein | 67.87 | |
| M366 A2 | H-P0397 | phosphoglycerate dehydrogenase | 57.75 | |
| | | (serA) | | |
| M366 B2 | H-P0398 | hypothetical protein | 20.13 | |
| M366 C2 | H-P0399 | ribosomal protein SI (rpsl) | 61.27 | |
| M366 D2 | H-P0403 | phenylalanyl-tRNA synthetase, | 36.19 | |
| | | alpha subunit (pheS) | | |
| M366 E2 | H-P0404 | protein kinase C inhibitor | 11.55 | |
| | | (SP: P16436) | | |
| M366 F2 | H-P0405 | nifS-like protein | 48.51 | |
| M366 G2 | H-P0406 | hypothetical protein | 21.67 | |
| M366 H2 | H-P0407 | biotin sulfoxide reductase (bisC) | 87.67 | |
| M381 D1 | H-P0409 | GMP synthase (guaA) | 55.99 | |
| M381 F1 | H-P0410 | putative neuraminyllactose- | 27.5 | |
| | | binding hemagglutinin homolog | | |
| | | (hpaA) | | |
| M366 A3 | H-P0411 | hypothetical protein | 11.66 | |
| M366 B3 | H-P0412 | hypothetical protein | 3.63 | |
| M366 C3 | H-P0413 | transposase-like protein, PS31S | 29.59 | |
| M366 D3 | H-P0414 | IS200 insertion sequence from | 15.29 | |
| | | SARA17 | | |
| M366 E3 | H-P0415 | conserved hypothetical integral | 68.64 | |
| | | membrane protein | | |
| M366 F3 | H-P0416 | cyclopropane fatty acid synthase | 42.9 | |
| | | (cfa) | | |
| M366 G3 | H-P0417 | methionyl-tRNA synthetase | 71.61 | |
| | | (metS) | | |
| M366 H3 | H-P0418 | hypothetical protein | 36.96 | |
| M366 A4 | H-P0419 | conserved hypothetical protein | 28.82 | |
| M366 B4 | H-P0420 | hypothetical protein | 15.73 | |
| M366 C4 | H-P0421 | type I capsular polysaccharide | 42.9 | |
| | | biosynthesis protein J (capJ) | | |
| M366 D4 | H-P0422 | arginine decarboxylase (speA) | 67.76 | |
| M366 E4 | H-P0424 | hypothetical protein | 68.2 | |
| M366 F4 | H-P0425 | hypothetical protein | 45.98 | |
| M366 G4 | H-P0427 | hypothetical protein | 12.32 | |
| M366 H4 | H-P0433 | hypothetical protein | 16.28 | |
| M366 A5 | H-P0436 | hypothetical protein | 13.42 | |
| M366 B5 | H-P0437 | IS605 transposase (tnpA) | 15.73 | |
| M366 C5 | H-P0438 | IS605 transposase (tnpB) | 47.08 | |
| M366 D5 | H-P0442 | hypothetical protein | 9.79 | |
| M366 E5 | H-P0445 | hypothetical protein | 6.82 | |
| M366 F5 | H-P0452 | hypothetical protein | 57.09 | |
| M366 G5 | H-P0455 | hypothetical protein | 11.44 | |
| M366 H5 | H-P0457 | hypothetical protein | 9.68 | |
| M366 A6 | H-P0463 | type I restriction enzyme M | 53.68 | |
| | | protein (hsdM) | | |
| M366 B6 | H-P0464 | type I restriction enzyme R | 116.16 | |
| | | protein (hsdR) | | |
| M366 C6 | H-P0465 | conserved hypothetical protein | 69.52 | |
| M366 D6 | H-P0466 | conserved hypothetical protein | 28.16 | |
| M366 E6 | H-P0467 | conserved hypothetical integral | 12.76 | |
| | | membrane protein | | |
| M366 F6 | H-P0468 | conserved hypothetical protein | 54.56 | |
| M366 G6 | H-P0469 | conserved hypothetical protein | 17.93 | |
| M366 H6 | H-P0471 | glutathione-regulated potassium- | 45.87 | |
| | | efflux system protein (kefB) | | |
| M366 A7 | H-P0472 | outer membrane protein (omp 11) | 20.57 | |
| M366 B7 | H-P0473 | molybdenum ABC transporter, | 27.17 | |
| | | periplasmic molybdate-binding | | |
| | | protein (modA) | | |
| M366 C7 | H-P0474 | molybdenum ABC transporter, | 24.75 | |
| | | permease protein (modB) | | |
| M366 D7 | H-P0475 | molybdenum ABC transporter, | 29.26 | |
| | | ATP-binding protein (modD) | | |
| M366 E7 | H-P0476 | glutamyl-tRNA synthetase (gltX) | 51.04 | |
| M366 F7 | H-P0477 | outer membrane protein (omp12) | 40.48 | |
| M366 G7 | H-P0478 | adenine specific DNA | 60.06 | |
| | | methyltransferase (VSPIM) | | |
| M366 H7 | H-P0479 | hypothetical protein | 31.13 | |
| M366 A8 | H-P0481 | adenine specific DNA | 23.32 | |
| | | methyltransferase (MFOKI) | | |
| M366 B8 | H-P0482 | hypothetical protein | 18.81 | |
| M366 C8 | H-P0483 | cytosine specific DNA | 36.3 | |
| | | methyltransferase (H-PHIMC) | | |
| M367 A1 | H-P0486 | hypothetical protein | 58.19 | |
| M367 B1 | H-P0487 | hypothetical protein | 52.91 | |
| M367 C1 | H-P0489 | hypothetical protein | 32.56 | |
| M367 D1 | H-P0490 | putative potassium channel | 41.69 | |
| | | protein, putative | | |
| M367 E1 | H-P0491 | ribosomal protein L28 (rpL28) | 6.93 | |
| M367 F1 | H-P0492 | hypothetical protein | 30.69 | |
| M367 G1 | H-P0494 | UDP-N-acetylmuramoylalanine- | 46.53 | |
| | | D-glutamate ligase (murD) | | |
| M367 H1 | H-P0495 | hypothetical protein | 9.57 | |
| M367 A2 | H-P0496 | conserved hypothetical protein | 14.74 | |
| M367 B2 | H-P0498 | sodium- and chloride-dependent | 48.73 | |
| | | transporter | | |
| M367 C2 | H-P0499 | phospholipase A1 precursor (DR- | 39.16 | |
| | | phospholipase A) | | |
| M367 D2 | H-P0500 | DNA polymerase III beta-subunit | 41.25 | |
| | | (dnaN) | | |
| M367 E2 | H-P0501 | DNA gyrase, sub B (gyrB) | 85.14 | |
| M367 F2 | H-P0503 | hypothetical protein | 27.17 | |
| M367 G2 | H-P0504 | hypothetical protein | 5.5 | |
| M367 H2 | H-P0505 | hypothetical protein | 17.05 | |
| M367 A3 | H-P0507 | conserved hypothetical protein | 23.43 | |
| M367 B3 | H-P0509 | glycolate oxidase subunit (glcD) | 50.6 | |
| M367 C3 | H-P0510 | dihydrodipicolinate reductase | 28.05 | |
| | | (dapB) | | |
| M367 D3 | H-P0512 | glutamine synthetase (glnA) | 53.02 | |
| M367 E3 | H-P0514 | ribosomal protein L9 (rpl9) | 16.61 | |
| M367 F3 | H-P0515 | heat shock protein (hsIV) | 19.91 | |
| M367 G3 | H-P0516 | heat shock protein (hsIU) ORFI | 48.84 | |
| M367 H3 | H-P0517 | GTP-binding protein (era) | 33.33 | |
| M367 A4 | H-P0519 | conserved hypothetical protein | 30.47 | |
| M367 B4 | H-P0520 | cag pathogenicity island protein | 12.76 | |
| | | (cag1) | | |
| M367 C4 | H-P0522 | cag pathogenicity island protein | 53.02 | |
| | | (cag3) | | |
| M367 D4 | H-P0523 | cag pathogenicity island protein | 18.7 | |
| | | (cag4) | | |
| M367 E4 | H-P0525 | virB11 homolog | 36.41 | |
| M367 F4 | H-P0526 | cag pathogenicity island protein | 22 | |
| | | (cag6) | | |
| M367 G4 | H-P0528 | cag pathogenicity island protein | 57.53 | |
| | | (cag8) | | |
| M379 H1 | H-P0531-2 | cag pathogenicity island protein | 24.09 | |
| | | (cag11) | | |
| M367 H4 | H-P0532 | cag pathogenicity island protein | 30.91 | |
| | | (cag12) | | |
| M367 A5 | H-P0534 | cag pathogenicity island protein | 21.67 | |
| | | (cag13) | | |
| M367 B5 | H-P0541 | cag pathogenicity island protein | 40.81 | |
| | | (cag20) | | |
| M367 C5 | H-P0542 | cag pathogenicity island protein | 15.73 | |
| | | (cag21) | | |
| M367 D5 | H-P0545 | cag pathogenicity island protein | 22.88 | |
| | | (cag24) | | |
| M367 E5 | H-P0549 | glutamate racemase (glr) | 28.16 | |
| M367 F5 | H-P0550 | transcription termination factor | 48.29 | |
| | | Rho (rho) | | |
| M367 G5 | H-P0551 | ribosomal protein L31 (rpl31) | 7.48 | |
| M367 H5 | H-P0552 | conserved hypothetical protein | 31.68 | |
| M367 A6 | H-P0553 | conserved hypothetical protein | 25.08 | |
| M367 B6 | H-P0554 | hypothetical protein | 35.42 | |
| M367 C6 | H-P0555 | hypothetical protein | 30.14 | |
| M367 D6 | H-P0556 | hypothetical protein | 16.06 | |
| M367 E6 | H-P0557 | acetyl-coenzyme A carboxylase | 34.43 | |
| | | (accA) | | |
| M367 F6 | H-P0558 | beta ketoacyl-acyl carrier protein | 45.43 | |
| | | synthase II (fabF) | | |
| M367 G6 | H-P0561 | 3-ketoacyl-acyl carrier protein | 27.28 | |
| | | reductase (fabG) | | |
| M367 H6 | H-P0562 | ribosomal protein S21 (rps21) | 7.81 | |
| M367 A7 | H-P0563 | hypothetical protein | 45.87 | |
| M367 B7 | H-P0566 | diaminopimelate epimerase | 30.14 | |
| | | (dapF) | | |
| M367 C7 | H-P0568 | hypothetical protein | 28.16 | |
| M367 D7 | H-P0570 | aminopeptidase a/i (pepA) | 54.67 | |
| M367 E7 | H-P0571 | conserved hypothetical integral | 21.23 | |
| | | membrane protein | | |
| M379 A2 | H-P0572-2 | adenine | 19.8 | |
| | | phosphoribosyltransferase (apt) | | |
| M379 B2 | H-P0573-2 | hypothetical protein | 12.21 | |
| M379 C2 | H-P0574-2 | galactosidase acetyltransferase | 16.72 | |
| | | (lacA) | | |
| M379 D2 | H-P0575-2 | conserved hypothetical membrane | 25.63 | |
| | | protein | | |
| M379 E2 | H-P0576-2 | signal peptidase I (lepB) | 32.01 | |
| M367 F7 | H-P0577 | methylene-tetrahydrofolate | 32.23 | |
| | | dehydrogenase (folD) | | |
| M367 G7 | H-P0579 | hypothetical protein | 20.35 | |
| M367 H7 | H-P0580 | hypothetical protein | 41.03 | |
| M367 A8 | H-P0581 | dihydroorotase (pyrC) | 37.4 | |
| M367 B8 | H-P0582 | hypothetical protein | 35.75 | |
| M367 C8 | H-P0583 | hypothetical protein | 32.34 | |
| M368 A1 | H-P0584 | flagellar switch protein (fliN) | 13.64 | |
| M368 B1 | H-P0585 | endonuclease III (nth) | 24.09 | |
| M368 C1 | H-P0587 | aminodeoxychorismate lyase | 36.3 | |
| | | (pabC) | | |
| M368 D1 | H-P0591 | ferredoxin oxidoreductase, | 20.57 | |
| | | gamma subunit | | |
| M368 E1 | H-P0593 | adenine specific DNA | 65.89 | |
| | | methyltransferase (mod) | | |
| M368 F1 | H-P0594 | hypothetical protein | 6.05 | |
| M368 G1 | H-P0596 | hypothetical protein | 21.23 | |
| M368 H1 | H-P0597 | penicillin-binding protein 1A | 72.6 | |
| | | (PBP-1A) | | |
| M368 A2 | H-P0599 | hemolysin secretion protein | 47.74 | |
| | | precursor (hylB) | | |
| M368 B2 | H-P0601 | flagellin A (flaA) | 56.21 | |
| M368 C2 | H-P0602 | endonuclease III | 24.09 | |
| M368 D2 | H-P0603 | hypothetical protein | 20.9 | |
| M379 F2 | H-P0608-2 | hypothetical protein | 17.71 | |
| M368 E2 | H-P0614 | hypothetical protein | 12.32 | |
| M368 F2 | H-P0616 | chemotaxis protein (cheV) | 34.54 | |
| M368 G2 | H-P06l7 | aspartyl-tRNA synthetase (aspS) | 63.58 | |
| M368 H2 | H-P0621 | DNA mismatch repair protein | 83.93 | |
| | | (MutS) | | |
| M368 A3 | H-P0622 | hypothetical protein | 13.31 | |
| M368 B3 | H-P0623 | UDP-N-acetylmuramate-alanine | 49.5 | |
| | | ligase (murC) | | |
| M368 C3 | H-P0625 | protein E (gcpE) | 39.6 | |
| M368 D3 | H-P0626 | tetrahydrodipicolinate N- | 44.22 | |
| | | succinyltransferase (dapD) | | |
| M368 E3 | H-P0627 | hypothetical protein | 12.21 | |
| M368 F3 | H-P0629 | hypothetical protein | 75.02 | |
| M368 G3 | H-P0630 | modulator of drug activity | 21.45 | |
| | | (mda66) | | |
| M368 H3 | H-P0631 | quinone-reactive Ni/Fe | 42.35 | |
| | | hydrogenase, small subunit | | |
| | | (hydA) | | |
| M368 A4 | H-P0632 | quinone-reactive Ni/Fe | 63.69 | |
| | | hydrogenase, large subunit | | |
| | | (hydB) | | |
| M368 B4 | H-P0633 | quinone-reactive Ni/Fe | 24.75 | |
| | | hydrogenase, cytochrome b | | |
| | | subunit (hydC) | | |
| M368 C4 | H-P0634 | quinone-reactive Ni/Fe | 19.69 | |
| | | hydrogenase (hydD) | | |
| M368 D4 | H-P0635 | hypothetical protein | 56.43 | |
| M368 E4 | H-P0636 | hypothetical protein | 10.23 | |
| M368 F4 | H-P0637 | hypothetical protein | 16.61 | |
| M368 G4 | H-P0638 | outer membrane protein (omp13) | 33.66 | |
| M368 H4 | H-P0643 | glutamyl-tRNA synthetase (gltX) | 48.4 | |
| M368 A5 | H-P0644 | conserved hypothetical integral | 10.78 | |
| | | membrane protein | | |
| M368 B5 | H-P0645 | soluble lytic murein | 61.71 | |
| | | transglycosylase (slt) | | |
| M368 C5 | H-P0646 | UDP-glucose pyrophosphorylase | 30.14 | |
| | | (galU) | | |
| M368 D5 | H-P0647 | hypothetical protein | 14.96 | |
| M368 E5 | H-P0648 | UDP-N-acetylglucosamine | 46.53 | |
| | | enolpyruvyl transferase (murZ) | | |
| M368 F5 | H-P0649 | aspartate ammonia-lyase (aspA) | 51.59 | |
| M368 G5 | H-P0650 | hypothetical protein | 21.67 | |
| M379 A3 | H-P0651-2 | fucosyltransferase | 52.47 | |
| M381 E3 | H-P0652 | phosphoserine phosphatase (serB) | 22.88 | |
| M368 H5 | H-P0653 | nonheme iron-containing ferritin | 18.48 | |
| | | (pfr) | | |
| M379 G2 | H-P0654-2 | conserved hypothetical protein | 39.71 | |
| M379 H2 | H-P0655-2 | protective surface antigen D15 | 100.87 | |
| M368 A6 | H-P0656 | conserved hypothetical protein | 42.24 | |
| M368 B6 | H-P0657 | processing protease (ymxG) | 47.63 | |
| M368 C6 | H-P0658 | PET112-like protein | 52.36 | |
| M368 D6 | H-P0659 | hypothetical protein | 45.65 | |
| M368 E6 | H-P0660 | hypothetical protein | 37.29 | |
| M368 F6 | H-P0661 | ribonuclease H (rnhA) | 15.84 | |
| M368 G6 | H-P0662 | ribonuclease III (rnc) | 26.51 | |
| M368 H6 | H-P0663 | chorismate synthase (aroC) | 40.26 | |
| M368 A7 | H-P0665 | oxygen-independent | 50.38 | |
| | | coproporphyrinogen III oxidase | | |
| | | (hemN) | | |
| M368 B7 | H-P0667 | hypothetical protein | 9.46 | |
| M368 C7 | H-P0668 | hypothetical protein | 66.88 | |
| M368 D7 | H-P0671 | outer membrane protein (omp14) | 29.81 | |
| M368 E7 | H-P0672 | solute-binding signature and | 43.01 | |
| | | mitochondrial signature protein | | |
| | | (aspB) | | |
| M379 B3 | H-P0673-2 | hypothetical protein | 46.97 | |
| M381 H3 | H-P0674 | hypothetical protein | 25.19 | |
| M368 F7 | H-P0676 | methylated-DNA-protein- | 18.59 | |
| | | cysteine methyltransferase (dat1) | | |
| M368 G7 | H-P0677 | conserved hypothetical integral | 28.16 | |
| | | membrane protein | | |
| M368 H7 | H-P0679 | lipopolysaccharide biosynthesis | 31.9 | |
| | | protein (wbpB) | | |
| M369 A1 | H-P0681 | hypothetical protein | 18.59 | |
| M369 B1 | H-P0682 | hypothetical protein | 13.97 | |
| M369 C1 | H-P0683 | UDP-N-acetylglucosamine | 47.74 | |
| | | pyrophosphorylase (glmU) | | |
| M369 D1 | H-P0685 | flagellar biosynthetic protein | 19.03 | |
| | | (fliP) | | |
| M369 E1 | H-P0687 | iron(II) transport protein (feoB) | 70.73 | |
| M369 F1 | H-P0688 | hypothetical protein | 18.37 | |
| M369 G1 | H-P0690 | acetyl coenzyme A | 43.12 | |
| | | acetyltransferase (thiolase) (fadA) | | |
| M381 A1 | H-P0691 | 3-oxoadipate coA-transferase | 25.63 | |
| | | subunit A (yxjD) | | |
| M381 B1 | H-P0692 | 3-oxoadipate coA-transferase | 22.88 | |
| | | subunit B (yxjE) | | |
| M369 H1 | H-P0694 | hypothetical protein | 28.38 | |
| M369 A2 | H-P0695 | hydantoin utilization protein A | 78.54 | |
| | | (hyuA) | | |
| M369 B2 | H-P0697 | hypothetical protein | 18.59 | |
| M369 C2 | H-P0699 | hypothetical protein | 37.73 | |
| M369 D2 | H-P0700 | diacylglycerol kinase (dgkA) | 14.19 | |
| M369 E2 | H-P0701 | DNA gyrase, sub A (gyrA) | 91.08 | |
| M369 F2 | H-P0703 | response regulator | 42.02 | |
| M369 G2 | H-P0707 | conserved hypothetical protein | 33.99 | |
| M369 H2 | H-P0711 | hypothetical protein | 44.77 | |
| M369 A3 | H-P0715 | ABC transporter, ATP-binding | 26.51 | |
| | | protein | | |
| M369 B3 | H-P0716 | conserved hypothetical protein | 14.74 | |
| M369 C3 | H-P0718 | conserved hypothetical integral | 23.21 | |
| | | membrane protein | | |
| M369 D3 | H-P0719 | hypothetical protein | 12.1 | |
| M369 E3 | H-P0723 | L-asparaginase II (ansB) | 36.41 | |
| M369 F3 | H-P0724 | anaerobic C4-dicarboxylate | 48.84 | |
| | | transport protein (dcuA) | | |
| M369 G3 | H-P0727 | transcriptional regulator, putative | 36.19 | |
| M369 H3 | H-P0728 | conserved hypothetical protein | 37.07 | |
| M369 A4 | H-P0730 | hypothetical protein | 11.22 | |
| M369 B4 | H-P0732 | hypothetical protein | 13.09 | |
| M369 C4 | H-P0734 | conserved hypothetical protein | 48.4 | |
| M369 D4 | H-P0735 | xanthine guanine phosphoribosyl | 16.94 | |
| | | transferase (gpt) | | |
| M369 E4 | H-P0737 | conserved hypothetical integral | 17.49 | |
| | | membrane protein | | |
| M381 H2 | H-P0738 | D-alanine: D-alanine ligase A | 38.28 | |
| | | (ddlA) | | |
| M369 F4 | H-P0739 | 2-hydroxy-6-oxohepta-2,4- | 26.62 | |
| | | dienoate hydrolase | | |
| M369 G4 | H-P0741 | conserved hypothetical protein | 17.82 | |
| M369 H4 | H-P0745 | conserved hypothetical protein | 36.08 | |
| M369 A5 | H-P0747 | conserved hypothetical protein | 43.34 | |
| M369 B5 | H-P0748 | cell division protein (ftsE) | 24.64 | |
| M369 C5 | H-P0749 | cell division membrane protein | 29.59 | |
| | | (ftsX) | | |
| M369 D5 | H-P0750 | hypothetical protein | 44.11 | |
| M369 E5 | H-P0752 | flagellar hook-associated protein | 74.25 | |
| | | 2 (fliD) | | |
| M381 F3 | H-P0755 | molybdopterin biosynthesis | 23.21 | |
| | | protein (moeB) | | |
| M379 C3 | H-P0757-2 | beta-alanine synthetase homolog | 32.23 | |
| M369 F5 | H-P0758 | conserved hypothetical integral | 48.18 | |
| | | membrane protein | | |
| M369 G5 | H-P0759 | conserved hypothetical integral | 45.98 | |
| | | membrane protein | | |
| M369 H5 | H-P0761 | hypothetical protein | 22.11 | |
| M369 A6 | H-P0762 | hypothetical protein | 20.46 | |
| M369 B6 | H-P0767 | hypothetical protein | 2.75 | |
| M369 C6 | H-P0768 | molybdenum cofactor | 35.42 | |
| | | biosynthesis protein A (moaA) | | |
| M369 D6 | H-P0769 | molybdopterin-guanine | 22.22 | |
| | | dinucleotide biosynthesis protein | | |
| | | A (mobA) | | |
| M369 E6 | H-P0771 | hypothetical protein | 27.06 | |
| M369 F6 | H-P0772 | N-acetylmuramoyl-L-alanine | 48.51 | |
| | | amidase (amiA) | | |
| M369 G6 | H-P0773 | hypothetical protein | 40.04 | |
| M369 H6 | H-P0777 | uridine 5′-monophosphate (UMP) | 26.51 | |
| | | kinase (pyrH) | | |
| M370 A1 | H-P0782 | hypothetical protein | 50.16 | |
| M370 B1 | H-P0783 | hypothetical protein | 18.26 | |
| M370 C1 | H-P0792 | sigma-54 interacting protein | 55.77 | |
| M370 D1 | H-P0793 | polypeptide deformylase (def) | 19.25 | |
| M370 E1 | H-P0794 | ATP-dependent clp protease | 21.67 | |
| | | proteolytic component (clpP) | | |
| M370 F1 | H-P0796 | outer membrane protein (omp18) | 30.69 | |
| M379 G3 | H-P0797-2 | flagellar sheath adhesin hpaA | 28.71 | |
| M379 H3 | H-P0798-2 | molybdenum cofactor | 17.49 | |
| | | biosynthesis protein C (moaC) | | |
| M370 G1 | H-P0799 | molybdopterin biosynthesis | 19.47 | |
| | | protein (mog) | | |
| M370 H1 | H-P0800 | molybdopterin converting factor, | 16.06 | |
| | | subunit 2 (moaE | | |
| M379 A4 | H-P0801-2 | molybdopterin converting factor, | 8.25 | |
| | | subunit 1 (moaD) | | |
| M379 B4 | H-P0802-2 | GTP cyclohydrolase II (ribA) | 21.23 | |
| M379 D3 | H-P0803-2 | hypothetical protein | 30.8 | |
| M379 E3 | H-P0804-2 | GTP cyclohydrolase II/3,4- | 37.95 | |
| | | dihydroxy-2-butanone 4- | | |
| | | phosphate synthase (ribA, ribB) | | |
| M379 F3 | H-P0805-2 | lipooligosaccharide 5G8 epitope | 31.35 | |
| | | biosynthesis-associated protein | | |
| | | (lex2B) | | |
| M370 A2 | H-P0806 | hypothetical protein | 22.77 | |
| M379 C4 | H-P0807-2 | iron(III) dicitrate transport protein | 86.68 | |
| | | (fecA) | | |
| M370 B2 | H-P0808 | holo-acp synthase (acpS) | 13.2 | |
| M370 C2 | H-P0809 | hypothetical protein | 20.24 | |
| M370 D2 | H-P0810 | conserved hypothetical protein | 22.11 | |
| M370 E2 | H-P0811 | hypothetical protein | 11.99 | |
| M370 F2 | H-P0812 | hypothetical protein | 37.07 | |
| M370 G2 | H-P0813 | conserved hypothetical protein | 22.66 | |
| M370 H2 | H-P0814 | thiamin biosynthesis protein | 28.16 | |
| | | (thiF) | | |
| M370 A3 | H-P0815 | flagellar motor rotation protein | 28.38 | |
| | | (motA) | | |
| M370 B3 | H-P0831 | conserved hypothetical ATP | 21.67 | |
| | | binding protein | | |
| M379 D4 | H-P0832-2 | spermidine synthase (speE) | 28.93 | |
| M379 E4 | H-P0833-2 | hypothetical protein | 32.23 | |
| M370 C3 | H-P0834 | GTP-binding protein homologue | 50.49 | |
| | | (yphC) | | |
| M370 D3 | H-P0835 | histone-like DNA-binding protein | 10.45 | |
| | | HU (hup) | | |
| M370 E3 | H-P0836 | hypothetical protein | 13.2 | |
| M370 F3 | H-P0837 | hypothetical protein | 11.33 | |
| M370 G3 | H-P0838 | hypothetical protein | 22.66 | |
| M370 H3 | H-P0839 | outer membrane protein P1 | 64.68 | |
| | | (ompP1) | | |
| M370 A4 | H-P0840 | flaA1 protein | 36.74 | |
| M370 B4 | H-P0841 | pantothenate metabolism | 46.86 | |
| | | flavoprotein (dfp) | | |
| M370 C4 | H-P0843 | thiamin phosphate | 24.2 | |
| | | pyrophosphorylase/ | | |
| | | hyroxyethylthiazole | | |
| | | kinase (thiB) | | |
| M370 D4 | H-P0845 | thiamin phosphate | 30.14 | |
| | | pyrophosphorylase/ | | |
| | | hyroxyethylthiazole | | |
| | | kinase (thiM) | | |
| M370 E4 | H-P0850 | type I restriction enzyme M | 58.08 | |
| | | protein (hsdM) | | |
| M370 F4 | H-P0851 | conserved hypothetical integral | 25.08 | |
| | | membrane protein | | |
| M370 G4 | H-P0854 | GMP reductase (guaC) | 36.08 | |
| M370 H4 | H-P0858 | ADP-heptose synthase (rfaE) | 50.82 | |
| M370 A5 | H-P0859 | ADP-L-glycero-D-mannoheptose- | 36.41 | |
| | | 6-epimerase (rfaD) | | |
| M370 B5 | H-P0861 | hypothetical protein | 27.17 | |
| M370 C5 | H-P0862 | hypothetical protein | 24.64 | |
| M379 F4 | H-P0863-2 | hypothetical protein | 59.73 | |
| M370 D5 | H-P0865 | deoxyuridine 5′-triphosphate | 16.06 | |
| | | nucleotidohydrolase (dut) | | |
| M370 E5 | H-P0866 | transcription elongation factor | 18.15 | |
| | | GreA (greA) | | |
| M379 G4 | H-P0867-2 | lipid A disaccharide synthetase | 39.71 | |
| | | (lpxB) | | |
| M379 H4 | H-P0870-2 | flagellar hook (flgE) | 79.09 | |
| M370 F5 | H-P0871 | CDP-diglyceride hydrolase (cdh) | 26.95 | |
| M370 G5 | H-P0872 | alkylphosphonate uptake protein | 12.1 | |
| | | (phnA) | | |
| M370 H5 | H-P0873 | hypothetical protein | 7.92 | |
| M371 A1 | H-P0879 | hypothetical protein | 22.33 | |
| M371 B1 | H-P0883 | Holliday junction DNA helicase | 20.24 | |
| | | (ruvA) | | |
| M371 C1 | H-P0885 | virulence factor mviN protein | 50.82 | |
| | | (mviN) | | |
| M371 D1 | H-P0886 | cysteinyl-tRNA synthetase (cysS) | 51.26 | |
| M371 E1 | H-P0889 | iron(III) dicitrate ABC | 35.97 | |
| | | transporter, permease protein | | |
| | | (fecD) | | |
| M371 F1 | H-P0890 | conserved hypothetical protein | 28.27 | |
| M371 G1 | H-P0891 | conserved hypothetical protein | 19.25 | |
| M371 H1 | H-P0892 | conserved hypothetical protein | 10.01 | |
| M371 A2 | H-P0894 | conserved hypothetical protein | 9.79 | |
| M371 B2 | H-P0895 | hypothetical protein | 13.86 | |
| M371 C2 | H-P0896 | outer membrane protein (omp19) | 77.99 | |
| M371 D2 | H-P0897 | hypothetical protein | 22.99 | |
| M371 E2 | H-P0898 | hydrogenase expression/formation | 40.81 | |
| | | protein (hypD) | | |
| M371 F2 | H-P0899 | hydrogenase expression/formation | 8.58 | |
| | | protein (hypC) | | |
| M371 G2 | H-P0900 | hydrogenase expression/formation | 26.73 | |
| | | protein (hypB) | | |
| M371 H2 | H-P0905 | phosphotransacetylase (pta) | 24.64 | |
| M371 A3 | H-P0906 | hypothetical protein | 58.08 | |
| M371 B3 | H-P0907 | hook assembly protein, flagella | 33.22 | |
| | | (flgD) | | |
| M371 C3 | H-P0909 | hypothetical protein | 22.22 | |
| M371 D3 | H-P0912 | outer membrane protein (omp20) | 56.76 | |
| M371 E3 | H-P0913 | outer membrane protein (omp21) | 58.3 | |
| M371 F3 | H-P0914 | hypothetical protein | 56.65 | |
| M371 G3 | H-P0915 | iron-regulated outer membrane | 61.93 | |
| | | protein (frpB) | | |
| M371 H3 | H-P0916 | iron-regulated outer membrane | 27.5 | |
| | | protein (frpB) | | |
| M380 A1 | H-P0917-2 | hypothetical protein | 2.64 | |
| M371 A4 | H-P0918 | hypothetical protein | 15.84 | |
| M371 B4 | H-P0920 | conserved hypothetical integral | 25.41 | |
| | | membrane protein | | |
| M371 C4 | H-P0921 | glyceraldehyde-3-phosphate | 36.63 | |
| | | dehydrogenase (gap) | | |
| M371 D4 | H-P0923 | outer membrane protein (omp22) | 40.7 | |
| M371 E4 | H-P0925 | recombinational DNA repair | 21.34 | |
| | | protein (recR) | | |
| M371 F4 | H-P0927 | heat shock protein (htpX) | 35.97 | |
| M371 G4 | H-P0928 | GTP cyclohydrolase 1 (folE) | 19.91 | |
| M371 H4 | H-P0929 | geranyltranstransferase (ispA) | 33.44 | |
| M371 A5 | H-P0930 | stationary-phase survival protein | 29.48 | |
| | | (surE) | | |
| M371 B5 | H-P0931 | hypothetical protein | 16.17 | |
| M371 C5 | H-P0932 | hypothetical protein | 11.11 | |
| M371 D5 | H-P0933 | hypothetical protein | 22.11 | |
| M371 E5 | H-P0934 | conserved hypothetical protein | 27.72 | |
| M371 F5 | H-P0935 | hypothetical protein | 17.82 | |
| M371 G5 | H-P0936 | proline/betaine transporter (proP) | 42.9 | |
| M371 H5 | H-P0938 | hypothetical protein | 12.76 | |
| M371 A6 | H-P0939 | amino acid ABC transporter, | 26.18 | |
| | | permease protein (yckJ) | | |
| M371 B6 | H-P0940 | amino acid ABC transporter, | 28.27 | |
| | | periplasmic binding protein | | |
| | | (yckK) | | |
| M371 C6 | H-P0941 | alanine racemase, biosynthetic | 41.58 | |
| | | (alr) | | |
| M371 D6 | H-P0942 | D-alanine glycine permease | 49.61 | |
| | | (dagA) | | |
| M371 E6 | H-P0943 | D-amino acid dehydrogenase | 45.21 | |
| | | (dadA) | | |
| M371 F6 | H-P0944 | translation initiation inhibitor, | 13.86 | |
| | | putative | | |
| M371 G6 | H-P0946 | conserved hypothetical integral | 54.67 | |
| | | membrane protein | | |
| M371 H6 | H-P0947 | hypothetical protein | 13.31 | |
| M371 A7 | H-P0949 | conserved hypothetical secreted | 16.61 | |
| | | protein | | |
| M371 B7 | H-P0950 | acetyl-CoA carboxylase beta | 31.9 | |
| | | subunit (accD) | | |
| M371 C7 | H-P0951 | hypothetical protein | 22.66 | |
| M371 D7 | H-P0952 | conserved hypothetical integral | 24.09 | |
| | | membrane protein | | |
| M371 E7 | H-P0953 | hypothetical protein | 20.79 | |
| M371 F7 | H-P0955 | prolipoprotein diacylglyceryl | 31.35 | |
| | | transferase (lgt) | | |
| M371 G7 | H-P0956 | conserved hypothetical protein | 26.73 | |
| M371 H7 | H-P0957 | 3-deoxy-d-manno-octulosonic- | 43.34 | |
| | | acid transferase (kdtA) | | |
| M371 A8 | H-P0958 | hypothetical protein | 28.05 | |
| M371 B8 | H-P0960 | glycyl-tRNA synthetase, alpha | 33.44 | |
| | | subunit (glyQ) | | |
| M371 C8 | H-P0961 | glycerol-3-phosphate | 34.43 | |
| | | dehydrogenase (NAD(P)+) | | |
| M380 B1 | H-P0965-2 | hypothetical protein | 48.84 | |
| M371 D8 | H-P0966 | conserved hypothetical protein | 60.5 | |
| M380 F1 | H-P0968-2 | hypothetical protein | 2.42 | |
| M371 E8 | H-P0969 | cation efflux system protein | 112.31 | |
| | | (czcA) | | |
| M371 F8 | H-P0970 | nickel-cobalt-cadmium resistance | 39.6 | |
| | | protein (nccB) | | |
| M371 G8 | H-P0971 | hypothetical protein | 45.54 | |
| M371 H8 | H-P0972 | glycyl-tRNA synthetase, beta | 77.22 | |
| | | subunit (glyS) | | |
| M371 A9 | H-P0973 | hypothetical protein | 38.94 | |
| M380 C1 | H-P0974-2 | phosphoglycerate mutase (pgm) | 54.12 | |
| M380 D1 | H-P0975-2 | conserved hypothetical protein | 10.34 | |
| M380 E1 | H-P0976-2 | adenosylmethionine-8-amino-7- | 48.07 | |
| | | oxononanoate aminotransferase | | |
| | | (bioA) | | |
| M380 H1 | H-P0994-2 | hypothetical protein | 29.48 | |
| M380 G1 | H-P1000-2 | PARA protein | 24.09 | |
| M380 A2 | H-P1001-2 | hypothetical protein | 10.45 | |
| M380 B2 | H-P1002-2 | hypothetical protein | 43.45 | |
| M380 C2 | H-P1003-2 | hypothetical protein | 40.81 | |
| M380 D2 | H-P1004-2 | hypothetical protein | 30.14 | |
| M380 E2 | H-P1005-2 | hypothetical protein | 11.55 | |
| M380 F2 | H-P1006-2 | conjugal transfer protein (traG) | 19.58 | |
| M380 G2 | H-P1017-2 | amino acid permease (rocE) | 57.2 | |
| M380 H2 | H-P1042-2 | hypothetical protein | 38.39 | |
| M380 A3 | H-P1056-2 | hypothetical protein | 31.35 | |
| M380 B3 | H-P1075-2 | conserved hypothetical secreted | 48.29 | |
| | | protein | | |
| M373 A1 | H-P1076 | hypothetical protein | 18.92 | |
| M373 B1 | H-P1077 | nickel transport protein (nixA) | 36.52 | |
| M373 C1 | H-P1080 | conserved hypothetical integral | 20.9 | |
| | | membrane protein | | |
| M373 D1 | H-P1081 | hypothetical protein | 22.88 | |
| M373 E1 | H-P1082 | multidrug resistance protein | 60.72 | |
| | | (msbA) | | |
| M373 F1 | H-P1083 | hypothetical protein | 52.8 | |
| M373 G1 | H-P1084 | aspartate transcarbamoylase | 33.88 | |
| | | (pyrB) | | |
| M373 H1 | H-P1085 | hypothetical protein | 18.92 | |
| M373 A2 | H-P1086 | hemolysin (tly) | 25.96 | |
| M373 B2 | H-P1087 | riboflavin biosynthesis regulatory | 30.91 | |
| | | protein (ribC) | | |
| M373 C2 | H-P1088 | transketolase A (tktA) | 70.62 | |
| M373 D2 | H-P1091 | alpha-ketoglutarate permease | 46.97 | |
| | | (kgtP) | | |
| M373 E2 | H-P1092 | flagellar basal-body rod protein | 29.7 | |
| | | (flgG) | | |
| M373 F2 | H-P1096 | IS605 transposase (tnpA) | 15.73 | |
| M373 G2 | H-P1098 | conserved hypothetical secreted | 32.01 | |
| | | protein | | |
| M373 H2 | H-P1101 | glucose-6-phosphate | 46.86 | |
| | | dehydrogenase (g6pD) | | |
| M373 A3 | H-P1102 | glucose-6-phosphate 1- | 25.08 | |
| | | dehydrogenase (devB) | | |
| M373 B3 | H-P1103 | glucokinase (glk) | 37.07 | |
| M373 C3 | H-P1108 | pyruvate ferredoxin | 20.57 | |
| | | oxidoreductase, gamma subunit | | |
| M373 D3 | H-P1109 | pyruvate ferredoxin | 14.41 | |
| | | oxidoreductase, delta subunit | | |
| M373 E3 | H-P1110 | pyruvate ferredoxin | 44.88 | |
| | | oxidoreductase, alpha subunit | | |
| M373 F3 | H-P1111 | pyruvate ferredoxin | 34.65 | |
| | | oxidoreductase, beta subunit | | |
| M373 G3 | H-P1112 | adenylosuccinate lyase (purB) | 48.51 | |
| M380 C3 | H-P1113-2 | outer membrane protein (omp24) | 30.58 | |
| M373 H3 | H-P1117 | conserved hypothetical secreted | 28.27 | |
| | | protein | | |
| M373 A4 | H-P1120 | hypothetical protein | 15.95 | |
| M373 B4 | H-P1121 | cytosine specific DNA | 34.43 | |
| | | methyltransferase (BSP6IM) | | |
| M380 D3 | H-P1122-2 | hypothetical protein | 8.47 | |
| M373 C4 | H-P1123 | peptidyl-prolyl cis-trans | 20.46 | |
| | | isomerase, FKBP-type rotamase | | |
| | | (slyD) | | |
| M373 D4 | H-P1124 | hypothetical protein | 36.52 | |
| M373 E4 | H-P1125 | peptidoglycan associated | 19.8 | |
| | | lipoprotein precursor (omp18) | | |
| M373 F4 | H-P1126 | colicin tolerance-like protein | 45.98 | |
| | | (tolB) | | |
| M373 G4 | H-P1128 | hypothetical protein | 9.35 | |
| M373 H4 | H-P1129 | biopolymer transport protein | 14.74 | |
| | | (exbD) | | |
| M373 A5 | H-P1131 | ATP synthase F1, subunit epsilon | 13.75 | |
| | | (atpC) | | |
| M373 B5 | H-P1134 | ATP synthase F1, subunit alpha | 55.44 | |
| | | (atpA) | | |
| M373 C5 | H-P1135 | ATP synthase F1, subunit delta | 19.91 | |
| | | (atpH) | | |
| M373 D5 | H-P1137 | ATP synthase F0, subunit b | 15.95 | |
| | | (atpF) | | |
| M373 E5 | H-P1138 | plasmid replication-partition | 32.01 | |
| | | related protein | | |
| M373 F5 | H-P1139 | SpoOJ regulator (soj) | 29.15 | |
| M373 G5 | H-P1140 | biotin operon repressor/biotin | 23.43 | |
| | | acetyl coenzyme A carboxylase | | |
| | | synthetase (birA) | | |
| M373 H5 | H-P1141 | methionyl-tRNA | 33.44 | |
| | | formyltransferase (fmt) | | |
| M373 A6 | H-P1144 | hypothetical protein | 9.46 | |
| M373 B6 | H-P1145 | hypothetical protein | 11.44 | |
| M373 C6 | H-P1147 | ribosomal protein L19 (rpl19) | 13.09 | |
| M373 D6 | H-P1148 | tRNA (guanine-NI)- | 25.3 | |
| | | methyltransferase (trmD) | | |
| M373 E6 | H-P1149 | conserved hypothetical protein | 20.35 | |
| M380 F3 | H-P1150-2 | hypothetical protein | 12.76 | |
| M373 F6 | H-P1152 | signal recognition particle protein | 49.39 | |
| | | (ffh) | | |
| M380 G3 | H-P1153-2 | valyl-tRNA synthetase (valS) | 96.25 | |
| M380 E3 | H-P1157-2 | outer membrane protein (omp26) | 135.41 | |
| M373 G6 | H-P1158 | pyrroline-5-carboxylate reductase | 28.38 | |
| | | (proC) | | |
| M373 H6 | H-P1159 | cell filamentation protein (fic) | 19.58 | |
| M373 A7 | H-P1160 | conserved hypothetical protein | 15.51 | |
| M380 A4 | H-P1163-2 | hypothetical protein | 7.04 | |
| M373 B7 | H-P1165 | tetracycline resistance protein | 42.57 | |
| | | tetA(P), putative | | |
| M373 C7 | H-P1168 | carbon starvation protein (cstA) | 75.68 | |
| M373 D7 | H-P1169 | glutamine ABC transporter, | 23.98 | |
| | | permease protein (glnP) | | |
| M380 H3 | H-P1169-2 | glutamine ABC transporter, | 23.98 | |
| | | permease protein (glnP) | | |
| M374 A1 | H-P1170 | glutamine ABC transporter, | 24.64 | |
| | | permease protein (glnP) | | |
| M374 B1 | H-P1171 | glutamine ABC transporter, ATP- | 27.39 | |
| | | binding protein (glnQ) | | |
| M374 C1 | H-P1172 | glutamine ABC transporter, | 30.58 | |
| | | periplasmic glutamine-binding | | |
| | | protein (glnH) | | |
| M374 D1 | H-P1173 | hypothetical protein | 20.24 | |
| M374 E1 | H-P1174 | glucose/galactose transporter | 44.88 | |
| | | (gluP) | | |
| M374 F1 | H-P1175 | conserved hypothetical integral | 47.96 | |
| | | membrane protein | | |
| M374 G1 | H-P1177 | outer membrane protein (omp27) | 70.62 | |
| M374 H1 | H-P1178 | purine-nucleoside phosphorylase | 25.74 | |
| | | (deoD) | | |
| M374 A2 | H-P1179 | phosphopentomutase (deoB) | 45.54 | |
| M374 B2 | H-P1180 | pyrimidine nucleoside transport | 46.09 | |
| | | protein (nupC) | | |
| M374 C2 | H-P1183 | NA+/H+ antiporter (napA) | 42.24 | |
| M374 D2 | H-P1184 | conserved hypothetical integral | 50.6 | |
| | | membrane protein | | |
| M374 E2 | H-P1185 | conserved hypothetical integral | 43.12 | |
| | | membrane protein | | |
| M374 F2 | H-P1186 | carbonic anhydrase | 22.33 | |
| M374 G2 | H-P1187 | hypothetical protein | 42.46 | |
| M374 H2 | H-P1188 | hypothetical protein | 29.7 | |
| M374 A3 | H-P1189 | aspartate-semialdehyde | 38.17 | |
| | | dehydrogenase (asd) | | |
| M374 B3 | H-P1191 | ADP-heptose-lps | 38.5 | |
| | | heptosyltransferase II (rfaF) | | |
| M374 C3 | H-P1196 | ribosomal protein S7 (rps7) | 17.16 | |
| M374 D3 | H-P1200 | ribosomal protein L10 (rpl10) | 18.15 | |
| M374 E3 | H-P1201 | ribosomal protein L1 (rpl1) | 25.85 | |
| M374 F3 | H-P1202 | ribosomal protein L11 (rpl11) | 15.62 | |
| M374 G3 | H-P1203 | transcription termination factor | 19.47 | |
| | | NusG (nusG) | | |
| M380 B4 | H-P1205-2 | translation elongation factor EF- | 44 | |
| | | Tu (tufB) | | |
| M374 H3 | H-P1206 | multidrug resistance protein | 63.69 | |
| | | (hetA) | | |
| M374 A4 | H-P1207 | hypothetical protein | 24.53 | |
| M374 B4 | H-P1210 | serine acetyltransferase (cysE) | 18.92 | |
| M380 F4 | H-P1213-2 | polynucleotide phosphorylase | 75.79 | |
| | | (pnp) | | |
| M380 G4 | H-P1214-2 | conserved hypothetical protein | 26.51 | |
| M380 C4 | H-P1215-2 | hypothetical protein | 8.91 | |
| M380 D4 | H-P1216-2 | conserved hypothetical secreted | 72.71 | |
| | | protein | | |
| M380 E4 | H-P1217-2 | hypothetical protein | 17.6 | |
| M374 C4 | H-P1220 | ABC transporter, ATP-binding | 25.19 | |
| | | protein (yhcG) | | |
| M374 D4 | H-P1221 | conserved hypothetical protein | 25.85 | |
| M374 E4 | H-P1222 | D-lactate dehydrogenase (dld) | 104.39 | |
| M374 F4 | H-P1224 | uroporphyrinogen III cosynthase | 24.97 | |
| | | (hemD) | | |
| M374 G4 | H-P1225 | conserved hypothetical integral | 14.41 | |
| | | membrane protein | | |
| M374 H4 | H-P1226 | oxygen-independent | 38.83 | |
| | | coproporphyrinogen III oxidase | | |
| | | (hemN) | | |
| M380 H4 | H-P1227-2 | cytochrome c553 | 10.67 | |
| M380 A5 | H-P1228-2 | invasion protein (invA) | 17.16 | |
| M380 B5 | H-P1229-2 | aspartokinase (lysC) | 44.66 | |
| M374 A5 | H-P1230 | hypothetical protein | 19.91 | |
| M374 B5 | H-P1231 | DNA polymerase III delta prime | 24.09 | |
| | | subunit (holB) | | |
| M374 C5 | H-P1232 | dihydropteroate synthase (folP) | 41.91 | |
| M380 D5 | H-P1233-2 | hypothetical protein | 16.94 | |
| M374 D5 | H-P1234 | conserved hypothetical integral | 32.89 | |
| | | membrane protein | | |
| M374 E5 | H-P1235 | conserved hypothetical integral | 45.76 | |
| | | membrane protein | | |
| M374 F5 | H-P1236 | hypothetical protein | 20.24 | |
| M374 G5 | H-P1237 | carbamoyl-phosphate synthetase | 41.36 | |
| | | (pyrAa) | | |
| M374 H5 | H-P1240 | conserved hypothetical protein | 21.01 | |
| M380 C5 | H-P1241-2 | alanyl-tRNA synthetase (alaS) | 93.28 | |
| M374 A6 | H-P1242 | conserved hypothetical protein | 8.47 | |
| M380 H5 | H-P1243-2 | outer membrane protein (omp28) | 80.74 | |
| M374 B6 | H-P1244 | ribosomal protein S18 (rps18) | 9.46 | |
| M374 C6 | H-P1245 | single-strand DNA-binding | 19.8 | |
| | | protein (ssb) | | |
| M374 D6 | H-P1246 | ribosomal protein S6 (rps6) | 15.73 | |
| M380 A6 | H-P1247-2 | hypothetical protein | 37.51 | |
| M374 E6 | H-P1248 | virulence associated protein | 70.95 | |
| | | homolog (vacB) | | |
| M380 B6 | H-P1249-2 | shikimate 5-dehydrogenase (aroE) | 29.04 | |
| M380 E5 | H-P1251-2 | oligopeptide ABC transporter, | 38.39 | |
| | | permease protein (oppB) | | |
| M380 F5 | H-P1252-2 | oligopeptide ABC transporter, | 65.45 | |
| | | periplasmic oligopeptide-binding | | |
| | | protein (oppA) | | |
| M380 G5 | H-P1253-2 | tryptophanyl-tRNA synthetase | 37.4 | |
| | | (trpS) | | |
| M374 F6 | H-P1254 | biotin synthesis protein (bioC) | 26.51 | |
| M374 G6 | H-P1255 | protein translocation protein, low | 22.22 | |
| | | temperature (secG) | | |
| M374 H6 | H-P1256 | ribosome releasing factor (frr) | 20.46 | |
| M374 A7 | H-P1257 | orotate phosphoribosyltransferase | 22.22 | |
| | | (pyrE) | | |
| M374 B7 | H-P1258 | conserved hypothetical | 17.05 | |
| | | mitochondrial protein 4 | | |
| M374 C7 | H-P1260 | NADH-ubiquinone | 14.74 | |
| | | oxidoreductase, NQO7 subunit | | |
| | | (NQO7) | | |
| M374 D7 | H-P1262 | NADH-ubiquinone | 29.37 | |
| | | oxidoreductase, NQO5 subunit | | |
| | | (NQO5) | | |
| M374 E7 | H-P1263 | NADH-ubiquinone | 45.1 | |
| | | oxidoreductase, NQO4 subunit | | |
| | | (NQO4) | | |
| M380 C6 | H-P1264-2 | hypothetical protein | 8.47 | |
| M374 F7 | H-P1265 | hypothetical protein | 36.19 | |
| M375 A1 | H-P1268 | NADH-ubiquinone | 24.31 | |
| | | oxidoreductase, NQO9 subunit | | |
| | | (NQO9) | | |
| M375 B1 | H-P1275 | phosphomannomutase (algC) | 50.6 | |
| M375 C1 | H-P1277 | tryptophan synthase, alpha | 28.93 | |
| | | subunit (trpA) | | |
| M375 D1 | H-P1278 | tryptophan synthase, beta subunit | 43.34 | |
| | | (trpB) | | |
| M375 E1 | H-P1279 | anthranilate isomerase (trpC) | 49.83 | |
| M375 F1 | H-P1282 | anthranilate synthase component 1 | 55.11 | |
| | | (trpE) | | |
| M375 G1 | H-P1285 | conserved hypothetical secreted | 25.41 | |
| | | protein | | |
| M375 H1 | H-P1286 | conserved hypothetical secreted | 20.13 | |
| | | protein | | |
| M375 A2 | H-P1287 | transcriptional regulator (tenA) | 23.98 | |
| M375 B2 | H-P1288 | hypothetical protein | 14.63 | |
| M375 C2 | H-P1289 | hypothetical protein | 17.82 | |
| M375 D2 | H-P1290 | nicotinamide mononucleotide | 24.31 | |
| | | transporter (pnuC) | | |
| M375 E2 | H-P1291 | conserved hypothetical protein | 22.55 | |
| M375 F2 | H-P1292 | ribosomal protein L17 (rpl17) | 12.87 | |
| M375 G2 | H-P1293 | DNA-directed RNA polymerase, | 37.95 | |
| | | alpha subunit (rpoA) | | |
| M375 H2 | H-P1294 | ribosomal protein S4 (rps4) | 22.99 | |
| M375 A3 | H-P1295 | ribosomal protein S11 (rps11) | 14.52 | |
| M375 B3 | H-P1296 | ribosomal protein S13 (rps13) | 13.31 | |
| M380 D6 | H-P1298-2 | translation initiation factor EF-1 | 8.03 | |
| | | (infA) | | |
| M375 C3 | H-P1299 | methionine amino peptidase | 27.94 | |
| | | (map) | | |
| M375 D3 | H-P1302 | ribosomal protein S5 (rps5) | 16.94 | |
| M375 E3 | H-P1303 | ribosomal protein L18 (rpl18) | 13.2 | |
| M375 F3 | H-P1305 | ribosomal protein S8 (rps8) | 14.52 | |
| M375 G3 | H-P1307 | ribosomal protein L5 (rpl5) | 20.02 | |
| M375 H3 | H-P1308 | ribosomal protein L24 (rpl24) | 8.14 | |
| M375 A4 | H-P1309 | ribosomal protein L14 (rpl14) | 13.53 | |
| M375 B4 | H-P1310 | ribosomal protein S17 (rps17) | 9.57 | |
| M375 C4 | H-P1312 | ribosomal protein L16 (rpl16) | 15.62 | |
| M375 D4 | H-P1314 | ribosomal protein L22 (rpl22) | 13.53 | |
| M375 E4 | H-P1315 | ribosomal protein S19 (rps19) | 10.34 | |
| M375 F4 | H-P1318 | ribosomal protein L4 (rpl4) | 23.76 | |
| M375 G4 | H-P1319 | ribosomal protein L3 (rpl3) | 21.12 | |
| M375 H4 | H-P1320 | ribosomal protein S10 (rps10) | 11.55 | |
| M375 A5 | H-P1321 | conserved hypothetical ATP- | 41.58 | |
| | | binding protein | | |
| M375 B5 | H-P1322 | hypothetical protein | 22.22 | |
| M375 C5 | H-P1323 | ribonuclease HII (rnhB) | 23.1 | |
| M375 D5 | H-P1324 | hypothetical protein | 9.24 | |
| M375 E5 | H-P1326 | hypothetical protein | 13.86 | |
| M375 F5 | H-P1327 | hypothetical protein | 45.43 | |
| M375 G5 | H-P1328 | cation efflux system protein | 37.29 | |
| | | (czcA) | | |
| M375 H5 | H-P1330 | conserved hypothetical integral | 12.76 | |
| | | membrane protein | | |
| M375 A6 | H-P1331 | conserved hypothetical integral | 25.19 | |
| | | membrane protein | | |
| M375 B6 | H-P1332 | co-chaperone and heat shock | 40.7 | |
| | | protein (dnaJ) | | |
| M375 C6 | H-P1333 | hypothetical protein | 42.13 | |
| M375 D6 | H-P1335 | conserved hypothetical protein | 39.71 | |
| M375 E6 | H-P1336 | hypothetical protein | 27.94 | |
| M375 F6 | H-P1337 | conserved hypothetical protein | 19.25 | |
| M375 G6 | H-P1338 | conserved hypothetical protein | 16.39 | |
| M375 H6 | H-P1340 | biopolymer transport protein | 14.3 | |
| | | (exbD) | | |
| M375 A7 | H-P1341 | siderophore-mediated iron | 31.46 | |
| | | transport protein (tonB) | | |
| M375 B7 | H-P1342 | outer membrane protein (omp29) | 76.12 | |
| M375 C7 | H-P1343 | conserved hypothetical integral | 26.73 | |
| | | membrane protein | | |
| M375 D7 | H-P1344 | magnesium and cobalt transport | 35.09 | |
| | | protein (corA) | | |
| M375 E7 | H-P1345 | phosphoglycerate kinase | 44.33 | |
| M375 F7 | H-P1346 | glyceraldehyde-3-phosphate | 36.41 | |
| | | dehydrogenase (gap) | | |
| M375 G7 | H-P1347 | uracil-DNA glycosylase (ung) | 25.74 | |
| M375 H7 | H-P1349 | hypothetical protein | 42.68 | |
| M375 A8 | H-P1350 | protease | 50.6 | |
| M375 B8 | H-P1355 | nicotinate-nucleotide | 30.14 | |
| | | pyrophosphorylase (nadC) | | |
| M375 C8 | H-P1356 | quinolinate synthetase A (nadA) | 37.07 | |
| M375 D8 | H-P1357 | phosphatidylserine decarboxylase | 29.48 | |
| | | proenzyme (psd) | | |
| M375 E8 | H-P1358 | hypothetical protein | 18.59 | |
| M375 F8 | H-P1360 | 4-hydroxybenzoate | 32.45 | |
| | | octaprenyltransferase (ubiA) | | |
| M375 G8 | H-P1361 | competence locus E (comE3) | 45.98 | |
| M375 H8 | H-P1362 | replicative DNA helicase (dnaB) | 53.79 | |
| M375 A9 | H-P1363 | conserved hypothetical integral | 51.37 | |
| | | membrane protein | | |
| M376 A1 | H-P1364 | signal-transducing protein, | 43.78 | |
| | | histidine kinase | | |
| M376 B1 | H-P1365 | response regulator | 23.54 | |
| M376 C1 | H-P1371 | type III restriction enzyme R | 106.59 | |
| | | protein | | |
| M376 D1 | H-P1372 | rod shape-determining protein | 27.39 | |
| | | (mreC) | | |
| M376 E1 | H-P1373 | rod shape-determining protein | 38.28 | |
| | | (mreB) | | |
| M376 F1 | H-P1374 | ATP-dependent protease ATPase | 49.17 | |
| | | subunit (clpX) | | |
| M376 G1 | H-P1375 | UDP-N-acetylglucosamine | 29.81 | |
| | | acyltransferase (lpxA) | | |
| M376 H1 | H-P1376 | (3R)-hydroxymyristoyl-(acyl | 17.6 | |
| | | carrier protein) dehydratase | | |
| | | (fabZ) | | |
| M376 A2 | H-P1377 | hypothetical protein | 16.17 | |
| M376 B2 | H-P1378 | competence lipoprotein (comL) | 24.31 | |
| M376 C2 | H-P1379 | ATP-dependent protease (lon) | 91.96 | |
| M376 D2 | H-P1380 | prephenate dehydrogenase (tyrA) | 29.26 | |
| M381 C1 | H-P1381 | hypothetical protein | 8.58 | |
| M376 E2 | H-P1382 | hypothetical protein | 14.41 | |
| M376 F2 | H-P1383 | restriction modification system S | 17.71 | |
| | | subunit | | |
| M376 G2 | H-P1384 | hypothetical protein | 7.59 | |
| M376 H2 | H-P1385 | fructose-1,6-bisphosphatase | 32.01 | |
| M376 A3 | H-P1386 | D-ribulose-5-phosphate 3 | 23.98 | |
| | | epimerase (rpe) | | |
| M376 B3 | H-P1388 | hypothetical protein | 16.5 | |
| M376 C3 | H-P1389 | hypothetical protein | 6.71 | |
| M376 D3 | H-P1390 | hypothetical protein | 18.37 | |
| M376 E3 | H-P1391 | hypothetical protein | 10.89 | |
| M376 F3 | H-P1392 | fibronectin/fibrinogen-binding | 47.96 | |
| | | protein | | |
| M376 G3 | H-P1393 | DNA repair protein (recN) | 57.75 | |
| M376 H3 | H-P1394 | conserved hypothetical protein | 31.35 | |
| M376 A4 | H-P1395 | outer membrane protein (omp30) | 26.73 | |
| M376 B4 | H-P1396 | hypothetical protein | 31.79 | |
| M376 C4 | H-P1398 | alanine dehydrogenase (ald) | 41.91 | |
| M376 D4 | H-P1399 | arginase (rocF) | 35.53 | |
| M376 E4 | H-P1400 | iron(III) dicitrate transport protein | 92.73 | |
| | | (fecA) | | |
| M376 F4 | H-P1401 | conserved hypothetical protein | 25.96 | |
| M381 A2 | H-P1402 | type I restriction enzyme R | 109.34 | |
| | | protein (hsdR) | | |
| M381 B2 | H-P1403 | type I restriction enzyme M | 89.98 | |
| | | protein (hsdM) | | |
| M376 G4 | H-P1405 | hypothetical protein | 3.85 | |
| M376 H4 | H-P1406 | biotin synthetase (bioB) | 31.13 | |
| M376 A5 | H-P1407 | conserved hypothetical integral | 32.23 | |
| | | membrane protein | | |
| M381 C2 | H-P1408 | hypothetical protein | 12.32 | |
| M381 D2 | H-P1409 | hypothetical protein | 63.69 | |
| M376 B5 | H-P1410 | hypothetical protein | 43.45 | |
| M376 C5 | H-P1411 | hypothetical protein | 68.2 | |
| M376 D5 | H-P1412 | hypothetical protein | 33.99 | |
| M376 E5 | H-P1413 | conserved hypothetical protein | 16.39 | |
| M376 F5 | H-P1414 | conserved hypothetical protein | 12.54 | |
| M376 G5 | H-P1415 | tRNA delta(2)- | 29.37 | |
| | | isopentenylpyrophosphate | | |
| | | transferase (miaA) | | |
| M376 H5 | H-P1418 | UDP-N- | 28.6 | |
| | | acetylenolpyruvoylglucosamine | | |
| | | reductase (murB) | | |
| M376 A6 | H-P1419 | flagellar biosynthetic protein | 9.79 | |
| | | (fliQ) | | |
| M376 B6 | H-P1420 | flagellar export protein ATP | 47.85 | |
| | | synthase (fliI) | | |
| M376 C6 | H-P1421 | conjugative transfer regulon | 33.55 | |
| | | protein (trbB) | | |
| M376 D6 | H-P1423 | conserved hypothetical protein | 9.35 | |
| M376 E6 | H-P1424 | hypothetical protein | 22.77 | |
| M376 F6 | H-P1425 | hypothetical protein | 8.36 | |
| M376 G6 | H-P1427 | histidine-rich, metal binding | 6.71 | |
| | | polypeptide (hpn) | | |
| M376 H6 | H-P1428 | conserved hypothetical protein | 39.38 | |
| M376 A7 | H-P1429 | polysialic acid capsule expression | 36.3 | |
| | | protein (kpsF) | | |
| M376 B7 | H-P1430 | conserved hypothetical ATP- | 75.9 | |
| | | binding protein | | |
| M376 C7 | H-P1431 | 16S rRNA (adenosine-N6, N6-)- | 29.92 | |
| | | dimethyltransferase (ksgA) | | |
| M376 D7 | H-P1432 | histidine and glutamine-rich | 8.03 | |
| | | protein | | |
| M376 E7 | H-P1433 | hypothetical protein | 94.27 | |
| M376 F7 | H-P1434 | formyltetrahydrofolate hydrolase | 32.34 | |
| | | (purU) | | |
| M376 G7 | H-P1435 | protease IV (PspA) | 32.23 | |
| M376 H7 | H-P1436 | hypothetical protein | 9.13 | |
| M376 A8 | H-P1438 | conserved hypothetical | 37.29 | |
| | | lipoprotein | | |
| M376 B8 | H-P1439 | hypothetical protein | 9.02 | |
| M376 C8 | H-P1440 | hypothetical protein | 28.6 | |
| M376 D8 | H-P1441 | peptidyl-prolyl cis-trans | 18.04 | |
| | | isomerase B, cyclosporin-type | | |
| | | rotamase (ppi) | | |
| M376 E8 | H-P1442 | carbon storage regulator (csrA) | 8.47 | |
| M376 F8 | H-P1443 | conserved hypothetical protein | 29.59 | |
| M376 G8 | H-P1444 | small protein (smpB) | 16.83 | |
| M376 H8 | H-P1445 | biopolymer transport protein | 16.61 | |
| | | (exbB) | | |
| M376 A9 | H-P1446 | biopolymer transport protein | 14.74 | |
| | | (exbD) | | |
| M376 B9 | H-P1447 | ribosomal protein L34 (rpl34) | 4.95 | |
| M376 C9 | H-P1448 | ribonuclease P, protein | 17.82 | |
| | | component (rnpA) | | |
| M376 D9 | H-P1449 | conserved hypothetical protein | 12.98 | |
| M376 E9 | H-P1450 | 60 kDa inner-membrane protein | 60.28 | |
| M376 F9 | H-P1451 | hypothetical protein | 29.15 | |
| M376 G9 | H-P1452 | thiophene and furan oxidizer | 50.82 | |
| | | (tdhF) | | |
| M376 H9 | H-P1453 | conserved hypothetical protein | 82.17 | |
| M376 A10 | H-P1454 | hypothetical protein | 33.44 | |
| M376 B10 | H-P1455 | hypothetical protein | 14.41 | |
| M376 C10 | H-P1456 | membrane-associated lipoprotein | 19.36 | |
| | | (lpp20) | | |
| M376 D10 | H-P1457 | hypothetical protein | 23.21 | |
| M376 E10 | H-P1458 | thioredoxin | 11.55 | |
| M376 F10 | H-P1461 | cytochrome c551 peroxidase | 38.61 | |
| M377 A1 | H-P1462 | secreted protein involved in | 19.03 | |
| | | flagellar motility | | |
| M377 B1 | H-P1463 | hypothetical protein | 24.86 | |
| M377 C1 | H-P1464 | conserved hypothetical secreted | 29.92 | |
| | | protein | | |
| M377 D1 | H-P1465 | ABC transporter, ATP-binding | 28.82 | |
| | | protein (H11087) | | |
| M377 E1 | H-P1466 | conserved hypothetical integral | 41.58 | |
| | | membrane protein | | |
| M377 F1 | H-P1467 | hypothetical protein | 25.52 | |
| M377 G1 | H-P1468 | branched-chain-amino-acid | 37.51 | |
| | | aminotransferase (ilvE) | | |
| M377 H1 | H-P1469 | outer membrane protein (omp31) | 27.39 | |
| M377 A2 | H-P1473 | hypothetical protein | 21.12 | |
| M377 B2 | H-P1474 | thymidylate kinase (tmk) | 21.12 | |
| M377 C2 | H-P1475 | lipopolysaccharide core | 17.38 | |
| | | biosynthesis protein (kdtB) | | |
| M377 D2 | H-P1476 | phenylacrylic acid decarboxylase | 20.68 | |
| M377 E2 | H-P1479 | hypothetical protein | 92.95 | |
| M377 F2 | H-P1480 | seryl-tRNA synthetase (serS) | 45.76 | |
| M377 G2 | H-P1481 | hypothetical protein | 29.26 | |
| M377 H2 | H-P1482 | hypothetical protein | 9.57 | |
| M377 A3 | H-P1483 | gerC2 protein (gerC2) | 27.17 | |
| M377 B3 | H-P1484 | conserved hypothetical integral | 16.39 | |
| | | membrane protein | | |
| M377 C3 | H-P1485 | proline dipeptidase (pepQ) | 21.01 | |
| M377 D3 | H-P1486 | conserved hypothetical integral | 41.47 | |
| | | membrane protein | | |
| M377 E3 | H-P1487 | conserved hypothetical integral | 40.26 | |
| | | membrane protein | | |
| M377 F3 | H-P1488 | conserved hypothetical secreted | 36.3 | |
| | | protein | | |
| M377 G3 | H-P1489 | lipase-like protein | 56.21 | |
| M381 G1 | H-P1490 | hemolysin | 49.5 | |
| M377 H3 | H-P1491 | phosphate permease | 58.74 | |
| M377 A4 | H-P1492 | conserved hypothetical nifU-like | 9.9 | |
| | | protein | | |
| M377 B4 | H-P1493 | hypothetical protein | 22.44 | |
| M377 C4 | H-P1494 | UDP-MurNac-tripeptide | 49.28 | |
| | | synthetase (murE) | | |
| M377 D4 | H-P1495 | transaldolase (tal) | 34.87 | |
| M377 E4 | H-P1496 | general stress protein (ctc) | 19.69 | |
| M377 F4 | H-P1497 | peptidyl-tRNA hydrolase (pth) | 20.57 | |
| M377 G4 | H-P1499 | hypothetical protein | 30.03 | |
| M377 H4 | H-P1501 | outer membrane protein (omp32) | 42.79 | |
| M377 A5 | H-P1502 | hypothetical protein | 16.06 | |
| M377 B5 | H-P1503 | cation-transporting ATPase, P- | 86.79 | |
| | | type (copA) | | |
| M377 C5 | H-P1504 | conserved hypothetical protein | 26.29 | |
| M377 D5 | H-P1505 | riboflavin biosynthesis protein | 37.95 | |
| | | (ribG) | | |
| M377 E5 | H-P1506 | glutamate permease (gltS) | 44.99 | |
| M377 F5 | H-P1507 | conserved hypothetical ATP- | 42.46 | |
| | | binding protein | | |
| M381 F2 | H-P1508 | ferrodoxin-like protein | 50.49 | |
| M377 G5 | H-P1509 | conserved hypothetical integral | 28.93 | |
| | | membrane protein | | |
| M377 H5 | H-P1510 | conserved hypothetical protein | 12.98 | |
| M377 A6 | H-P1511 | hypothetical protein | 11.99 | |
| M377 B6 | H-P1512 | iron-regulated outer membrane | 96.58 | |
| | | protein (frpB) | | |
| M377 C6 | H-P1513 | selenocystein synthase (selA) | 42.57 | |
| M377 D6 | H-P1514 | transcription termination factor | 43.56 | |
| | | NusA (nusA) | | |
| M377 E6 | H-P1518 | hypothetical protein | 10.56 | |
| M381 B3 | H-P1521 | type III restriction enzyme R | 106.48 | |
| | | protein (res) | | |
| M381 C3 | H-P1523 | DNA recombinase (recG) | 68.64 | |
| M377 F6 | H-P1524 | hypothetical protein | 12.76 | |
| M377 G6 | H-P1525 | hypothetical protein | 23.32 | |
| M377 H6 | H-P1526 | exodeoxyribonuclease (lexA) | 27.61 | |
| M377 A7 | H-P1527 | hypothetical protein | 52.8 | |
| M377 B7 | H-P1530 | purine nucleoside phosphorylase | 19.91 | |
| | | (punB) | | |
| M377 C7 | H-P1531 | hypothetical protein | 8.8 | |
| M377 D7 | H-P1532 | glucosamine fructose-6-phosphate | 65.78 | |
| | | aminotransferase (isomerizing) | | |
| | | (glmS) | | |
| M377 E7 | H-P1533 | conserved hypothetical protein | 25.52 | |
| M377 F7 | H-P1534 | IS605 transposase (tnpB) | 47.08 | |
| M377 G7 | H-P1535 | IS605 transposase (tnpA) | 15.73 | |
| M377 H7 | H-P1541 | transcription-repair coupling | 110 | |
| | | factor (trcF) | | |
| M377 A8 | H-P1548 | conserved hypothetical integral | 12.43 | |
| | | membrane protein | | |
| M377 B8 | H-P1551 | conserved hypothetical secreted | 14.08 | |
| | | protein | | |
| M377 C8 | H-P1552 | Na+/H+ antiporter (nhaA) | 48.29 | |
| M381 B4 | H-P1554 | ribosomal protein S2 (rps2) | 29.15 | |
| M381 D4 | H-P1555 | translation elongation factor EF- | 39.16 | |
| | | Ts (tsf) | | |
| M377 D8 | H-P1556 | cell division protein (ftsl) | 67.76 | |
| M381 E4 | H-P1557 | flagellar basal-body protein (fliE) | 12.1 | |
| M381 F4 | H-P1558 | flagellar basal-body rod protein | 17.82 | |
| | | (flgC) (proximal rod protein) | | |
| M381 G4 | H-P1559 | flagellar basal-body rod protein | 15.51 | |
| | | (flgB) (proximal rod protein) | | |
| M378 A1 | H-P1560 | cell division protein (ftsW) | 42.79 | |
| M378 B1 | H-P1561 | iron(III) ABC transporter, | 36.96 | |
| | | periplasmic iron-binding protein | | |
| | | (ceuE) | | |
| M378 C1 | H-P1562 | iron(III) ABC transporter, | 36.74 | |
| | | periplasmic iron-binding protein | | |
| | | (ceuE) | | |
| M378 D1 | H-P1563 | alkyl hydroperoxide reductase | 21.89 | |
| | | (tsaA) | | |
| M378 E1 | H-P1564 | outer membrane protein | 29.92 | |
| M378 F1 | H-P1565 | penicillin-binding protein 2 | 64.79 | |
| | | (pbp2) | | |
| M378 G1 | H-P1566 | hypothetical protein | 16.28 | |
| M378 H1 | H-P1567 | conserved hypothetical ATP- | 22.99 | |
| | | binding protein | | |
| M378 A2 | H-P1568 | hypothetical protein | 20.24 | |
| M378 B2 | H-P1569 | hypothetical protein | 21.78 | |
| M378 C2 | H-P1570 | conserved hypothetical protein | 18.15 | |
| M378 D2 | H-P1571 | rare lipoprotein A (rlpA) | 34.76 | |
| M378 E2 | H-P1572 | regulatory protein DniR | 41.03 | |
| M378 F2 | H-P1573 | conserved hypothetical protein | 28.05 | |
| M378 G2 | H-P1576 | ABC transporter, ATP-binding | 36.08 | |
| | | protein (abc) | | |
| M378 H2 | H-P1577 | ABC transporter, permease | 23.76 | |
| | | protein (yaeE) | | |
| M378 A3 | H-P1580 | hypothetical protein | 24.31 | |
| M378 B3 | H-P1581 | methicillin resistance protein | 37.07 | |
| | | (llm) | | |
| M378 C3 | H-P1582 | pyridoxal phosphate biosynthetic | 28.93 | |
| | | protein J (pdxJ) | | |
| M378 D3 | H-P1583 | pyridoxal phosphate biosynthetic | 33.88 | |
| | | protein A (pdxA) | | |
| M378 E3 | H-P1584 | sialoglycoprotease (gcp) | 37.51 | |
| M378 F3 | H-P1585 | flagellar basal-body rod protein | 28.93 | |
| | | (flgG) | | |
| M378 G3 | H-P1587 | conserved hypothetical protein | 17.16 | |
| M378 H3 | H-P1588 | conserved hypothetical protein | 27.94 | |
| M381 H1 | H-P1590 | hypothetical protein | 4.4 | |
| M318 G2 | H-S38729 | autoimmune antigen Ku, p70 | 67.1 | 67 |
| | | subunit | | |
| H1 | H-S39329 | Kallikrein 1 | 24.64 | 30 |
| | | (renal/pancreas/salivary) | | |
| | | {alternative products} | | |
| M270 G4 | H-S43855 | Recoverin, photoreceptor protein | 22.11 | 32.0 kDa |
| M300 C2 | H-S56151 | milk fat globule protein HMFG | 24.09 | 30 |
| M318 C1 | H-S57153 | retinoblastoma-binding protein 1, | 101.31 | 101 |
| | | isoform 1 [RBBP1] | | |
| M271 B2 | H-S57162 | retinoblastoma-binding protein 1, | 93.72 | 110 |
| | | isoform III [RBBP1], | | |
| | | INTERACTS WITH THE VIRAL | | |
| | | PROTEIN-BINDING DOMAIN | | |
| | | OF THE RETINOBLASTOMA | | |
| | | PROTEIN. | | |
| M317 H3 | H-S62027 | transducin, gamma subunit | 8.25 | 11 |
| M270 G6 | H-S66793 | arrestin, X-anestin = S-antigen | 42.79 | 50.0 kDa |
| | | homolog [human, retina, mRNA, | | |
| | | 1314 nt], MAY PLAY A ROLE | | |
| | | IN AN AS YET UNDEFINED | | |
| | | RETINA-SPECIFIC SIGNAL | | |
| | | TRANSDUCTION. | | |
| M419 C2 | H-S67859 | “transcription initiation factor Ile. | 48.360 | 64.0 kDa |
| | | alpha subunit” | | |
| M302 D7 | H-S69022 | myosin, light polypeptide 2, | 18.26 | 31 |
| | | ventricular | | |
| H5 | H-S69272 | cytoplasmic antiproteinase = 38 | 41.47 | 50 |
| | | kda intracellular serine proteinase | | |
| | | inhibitor [human, placenta, | | |
| | | mRNA, 1465 nt] | | |
| D1 | H-S72043 | GIF = growth inhibitory factor | 7.59 | 19 |
| | | [human, brain, Genomic, 2015 nt] | | |
| M266 B3 | H-S74221 | cytokine IK factor | 17.93 | 36.0 kDa |
| D1 | H-S74445 | cellular retinoic acid-binding | 15.18 | 23 |
| | | protein [human, skin, mRNA, 735 | | |
| | | nt] | | |
| E3 | H-S74728 | antiquitin = 26 g turgor protein | 56.32 | 53 |
| | | homolog [human, kidney, mRNA, | | |
| | | 1809 nt] | | |
| D4 | H-S75174 | E2F transcription factor 4, | 45.87 | 58 |
| | | p107/p130-binding | | |
| 166-61 | H-S76474 | “trkB {alternately spliced} | 55 | 52.54 |
| | | [human, brain, mRNA]” | | |
| 169-40 | H-S76617 | “Blk = protein tyrosine kinase | 60 | 55.62 |
| | | [human, B lymphocytes, mRNA, | | |
| | | 2608 nt]” | | |
| M250 D3 | H-S79522 | ubiquitin carboxyl-terminal | 17.27 | 17.0 kDa |
| | | extension protein, Ubiquitin A-52 | | |
| | | residue ribosomal protein fusion | | |
| | | product 1 | | |
| M236 B4 | H-S80562 | calponin, acidic | 36.3 | 49 |
| G1 | H-S82470 | BB1 = malignant cell expression- | 37.73 | 34 |
| | | enhanced gene/tumor | | |
| | | progression-enhanced gene | | |
| | | [human, UM-UC-9 bladder | | |
| | | carcinoma cell line, mRNA, 1897 | | |
| | | nt] | | |
| M313 E1 | H-S85655 | prohibitin [PHB] | 30.03 | 40.0 kDa |
| M465 A6 | H-S87759 | protein phosphatase 2C alpha | 42.13 | 52.0 kDa |
| | | [human, teratocarcinoma, mRNA, | | |
| | | 2346 nt] | | |
| M472 B1 | H-U00803 | tyrosine-protein kinase FRK | 55.620 | 64.0 kDa |
| B2 | H-U02390 | Human adenylyl cyclase- | 52.58 | 55 |
| | | associated protein homolog CAP2 | | |
| | | (CAP2) mRNA, complete cds | | |
| 167-2 | H-U02680 | human protein tyrosine kinase | 36 | 38.57 |
| | | mRNA | | |
| G2 | H-U03056 | Human tumor suppressor (LUCA- | 47.96 | 47 |
| | | 1) mRNA, complete cds | | |
| M512 E3 | H-U03100 | Human alpha2(E)-catenin mRNA, | 102.52 | 102.0 kDa |
| | | complete cds | | |
| M306 G3 | H-U03187 | | 72.93 | 95.0 kDa |
| H3 | H-U03398 | Human receptor 4-1BB ligand | 28.05 | 51 |
| | | mRNA, complete cds | | |
| D3 | H-U03486 | Human connexin40 gene, | 39.49 | 40 |
| | | complete cds | | |
| M300 C3 | H-U03643 | leukophysin | 25.96 | 34 |
| F5 | H-U03749 | Human chromogranin A (CHGA) | 50.38 | 50 |
| | | gene, promoter and | | |
| M314 C3 | H-U03886 | GS2 (GB: U03886) | 27.94 | 32.0 kDa |
| M306 E3 | H-U04343 | CD86 antigen (CD28 antigen | 35.64 | 47 |
| | | ligand 2, B7-2 antigen) [CD86] | | |
| 167-61 | H-U05012 | TrkC | 92 | 90.82 |
| M302 G5 | H-U05340 | cell division cycle protein p55 | 55 | 55 |
| A4 | H-U05659 | Hydroxysteroid (17-beta) | 34.21 | 36 |
| | | dehydrogenase 3 | | |
| F1 | H-U05861 | Human hepatic dihydrodiol | 35.64 | 40 |
| | | dehydrogenase gene | | |
| M302 B2 | H-U06452 | antigen MART-1, melanoma | 13.09 | 20.0 kDa |
| 169-52 | H-U06454 | human AMP-activated protein | 70 | 60.79 |
| | | kinase (hAMPK) mRNA | | |
| M315 A3 | H-U06643 | lectin, epidermal | 15.07 | 18 |
| H1 | H-U06715 | Cytochrome B561 | 27.06 | 25 |
| M476 E5 | H-U07132 | Human steroid hormone receptor | 50.82 | 55.0 kDa |
| | | Ner-I mRNA, complete cds | | |
| M236 D3 | H-U07151 | guanine nucleotide-binding | 20.13 | 34 |
| | | protein ADP-ribosylation factor | | |
| | | like gene 3 | | |
| M317 G3 | H-U07559 | homeotic protein Islet-1 | 38.17 | 38 |
| M266 H1 | H-U07681 | Human NAD(H)-specific | 40.37 | 40 |
| | | isocitrate dehydrogenase alpha | | |
| | | subunit precursor mRNA, | | |
| | | complete cds | | |
| E3 | H-U07919 | Aldehyde dehydrogenase 6 | 56.43 | 53 |
| M298 A3 | H-U08021 | nicotinamide N-methyltransferase | 29.15 | 36.0 kDa |
| M297 B1 | H-U08024 | alcohol/hydroxysteroid | 31.46 | 50.0 kDa |
| | | sulfotransferase | | |
| A2 | H-U08336 | Human basic helix-loop-helix | 21.89 | 42 |
| | | transcription factor mRNA, | | |
| | | complete cds | | |
| E2 | H-U09303 | Human T cell leukemia LERK-2 | 38.17 | 40 |
| | | (EPLG2) mRNA, complete cds | | |
| M250 H5 | H-U09559 | RCHI, RAG (recombination | 58.3 | 58.0 kDa |
| | | activating gene) cohort 1 | | |
| 167-50 | H-U09564 | human serine kinase mRNA | 72 | 72.12 |
| 166-74 | H-U09578 | human MAPKAP kinase (3pK) | 50 | 42.09 |
| | | mRNA | | |
| M302 C4 | H-U09813 | ATP synthase, subunit 9, | 15.73 | 30 |
| | | mitochondrial | | |
| A1 | H-U09850 | Zinc finger protein 143 (clone | 68.97 | 68 |
| | | pHZ-1) | | |
| M423 E1 | H-U09937 | Human urokinase-type | 36.96 | 49.0 kDa |
| | | plasminogen receptor | | |
| M450 H4 | H-U10117 | Human endothelial-monocyte | 34.43 | 38.0 kDa |
| | | activating polypeptide II mRNA, | | |
| | | complete cds | | |
| M314 G1 | H-U10248 | ribosomal protein L29 | 17.6 | 27 |
| M298 H1 | H-U10323 | nuclear factor 45 | 44.77 | 45 |
| E1 | H-U10492 | Human Mox 1 protein (MOX1) | 28.05 | 37 |
| | | mRNA, complete cds | | |
| F3 | H-U10686 | Human MAGE-11 antigen | 35.2 | 35 |
| | | (MAGE11) gene, complete cds | | |
| 167-38 | H-U11050 | human NIMA-like protein kinase | 55 | 49.02 |
| | | 1 (NLK1) mRNA | | |
| M266 B2 | H-U11292 | Human Ki nuclear autoantigen | 29.48 | 32 |
| | | mRNA, complete cds, may play a | | |
| | | rol in cell adhesion | | |
| 167-62 | H-U11791 | human cyclin H m RNA | 40 | 35.60 |
| M423 D5 | H-U12255 | immunoglobulin gamma heavy | 40.26 | 48.0 kDa |
| | | chain Fc receptor R1, high affinity | | |
| M302 F7 | H-U12404 | Csa-19 | 23.98 | 32 |
| M236 A2 | H-U12465 | ribosomal protein L35 | 13.64 | 24 |
| 169-4 | H-U12535 | human epidermal growth factor | 100 | 90.49 |
| | | receptor kinase substrate (Eps8) | | |
| | | mRNA | | |
| F3 | H-U12597 | Human tumor necrosis factor type | 55.22 | 64 |
| | | 2 receptor associated protein | | |
| | | (TRAP3) mRNA, complete cds | | |
| M314 D1 | H-U12979 | transcriptional coactivator PC4 | 14.08 | 23 |
| M476 G4 | H-U13044 | GA-binding protein transcription | 50.05 | 53.0 kDa |
| | | factor, alpha subunit (60 kD) | | |
| M302 F3 | H-U13665 | cathepsin O (GB: U13665) | 36.3 | 50.0 kDa |
| M311 G4 | H-U13831 | cellular retinol binding protein II | 14.85 | 20.0 kDa |
| A2 | H-U13991 | Human TATA-binding protein | 24.09 | 34 |
| | | associated factor 30 kDa subunit | | |
| | | (tafII30) mRNA, complete cds | | |
| M416 A4 | H-U14187 | Human receptor tyrosine kinase | 26.29 | 29.0 kDa |
| | | ligand LERK-3 (EPLG3) mRNA, | | |
| | | complete cds | | |
| M250 A2 | H-U14188 | eph-related receptor tyrosine | 22.22 | 27 |
| | | kinase ligand 4 [EPLG4] | | |
| M302 D2 | H-U14193 | human TFIIA gamma subunit | 12.060 | 28.0 kDa |
| | | mRNA | | |
| M416 G1 | H-U14603 | Human protein-tyrosine | 18.48 | 30.0 kDa |
| | | phosphatase (HU-PP-1) mRNA, | | |
| | | partial sequence | | |
| E2 | H-U14747 | Visinin-like 1 | 21.12 | 25 |
| M266 D4 | H-U14966 | ribosomal protein L5 | 32.78 | 38 |
| M314 E2 | H-U14967 | ribosomal protein L21 | 17.71 | 29 |
| M266 F5 | H-U14968 | ribosomal protein L27a | 16.39 | 19.0 kDa |
| M248 E3 | H-U14969 | ribosomal protein L28 | 15.18 | 27 |
| M266 E1 | H-U14971 | ribosomal protein S9 | 21.45 | 30 |
| M250 C2 | H-U15009 | small nuclear ribonucleoprotein, | 13.97 | 17.0 kDa |
| | | Sm D3 | | |
| M311 D4 | H-U16660 | enoyl-Coenzyme A hydratase-like | 36.19 | 38 |
| | | protein, peroxisomal | | |
| M302 H4 | H-U17074 | cyclin-dependent kinase 6 | 18.59 | 29 |
| | | inhibitor p18 | | |
| M306 A2 | H-U17195 | A-kinase anchor protein 100 | 72.05 | 100 |
| | | [AKAP100*] | | |
| D1 | H-U17280 | Steroidogenic acute regulatory | 31.46 | 35 |
| | | protein | | |
| M316 F1 | H-U18291 | cell division cycle protein 16 | 68.2 | 71.0 kDa |
| C5 | H-U18420 | Human ras-related small GTP | 23.87 | 33 |
| | | binding protein Rab5 (rab5) | | |
| | | mRNA, complete cds | | |
| M311 A2 | H-U18423 | spinal muscular atrophy gene | 32.45 | 41 |
| M248 D4 | H-U18914 | hypothetical protein, (Human | 20.35 | 32 |
| | | 19.8 kDa protein mRNA, | | |
| | | complete cds) | | |
| M302 B5 | H-U19718 | microfibril-associated | 20.24 | 34.0 kDa |
| | | glycoprotein 2 | | |
| M305 E3 | H-U20240 | CCAAT/enhancer-binding protein | 16.61 | 29 |
| | | gamma | | |
| M302 A8 | H-U20352 | malate dehydrogenase | 36.85 | 40 |
| M416 F4 | H-U20391 | Human folate receptor (FOLR1) | 28.38 | 34.0 kDa |
| | | gene, complete cds | | |
| M311 D1 | H-U20536 | apoptotic cysteine protease Mch2 | 32.34 | 38.0 kDa |
| M431 G2 | H-U20659 | RNA polymerase II, subunit B7 | 19.03 | 31.0 kDa |
| M499 C1 | H-U20938 | Human lymphocyte | 112.86 | 100.0 kDa |
| | | dihydropyrimidine dehydrogenase | | |
| | | mRNA, complete cds | | |
| M305 F2 | H-U20972 | 14-3-3 protein, epsilon | 28.16 | 36 |
| M271 D3 | H-U21049 | hypothetical protein | 12.65 | 16 |
| | | (GB: U21049), ESTs, Highly | | |
| | | similar to DD96 [H. sapiens]. | | |
| M421 G5 | H-U21858 | Human transcriptional activation | 29.15 | 38.0 kDa |
| | | factor TAFII32 mRNA, complete | | |
| | | cds | | |
| M424 H3 | H-U22662 | Human nuclear orphan receptor | 49.28 | 49.0 kDa |
| | | LXR-alpha mRNA, complete cds | | |
| M271 D2 | H-U24074 | killer cell inhibitory receptor | 37.62 | 43 |
| | | [KIR], Homo sapiens natural | | |
| | | killer-associated transcript 3 | | |
| | | (NKAT3), complete cds. | | |
| | | RECEPTOR ON NATURAL | | |
| | | KILLER (NK) CELLS FOR | | |
| | | HLA-C ALLELES. | | |
| 169-29 | H-U24153 | human p21-activated protein | 60 | 57.82 |
| | | kinase (Pak2) gene | | |
| M385 H2 | H-U24166 | EB1 | 29.59 | 36.0 kDa |
| G1 | H-U24169 | Human JTV-1 (JTV-1) mRNA, | 34.43 | 40 |
| | | complete cds | | |
| E1 | H-U24576 | Human breast tumor autoantigen | 18.26 | 27 |
| | | mRNA, complete sequence | | |
| G4 | H-U24577 | Human LDL-phospholipase A2 | 48.62 | 52 |
| | | mRNA, complete cds | | |
| H1 | H-U25789 | Human ribosomal protein L21 | 17.71 | 32 |
| | | mRNA, complete cds | | |
| M416 D1 | H-U25849 | Human red cell-type low | 17.49 | 28.0 kDa |
| | | molecular weight acid | | |
| | | phosphatase (ACP1) gene, 5′ | | |
| | | flanking region and | | |
| M300 A3 | H-U26312 | heterochromatin protein H-P1Hs- | 19.14 | 30 |
| | | gamma | | |
| M416 D3 | H-U26403 | Human receptor tyrosine kinase | 25.19 | 30.0 kDa |
| | | ligand LERK-7 precursor | | |
| | | (EPLG7) mRNA, complete cds | | |
| M317 E2 | H-U27143 | human protein kinase C inhibitor- | 13.900 | 17.0 kDa |
| | | I cDNA | | |
| E5 | H-U28249 | Human 11 kd protein mRNA, | 12.32 | 12 |
| | | complete cds | | |
| F4 | H-U28386 | Human nuclear localization | 58.3 | 54 |
| | | sequence receptor hSRP1alpha | | |
| | | mRNA, complete cds | | |
| M423 E3 | H-U28694 | Chemokine (C-C) receptor 3 | 39.16 | 39.0 kDa |
| M266 G6 | H-U28963 | Gps2 | 36.08 | 36 |
| M306 D3 | H-U30610 | CD94 antigen (NK/T-cell C-type | 19.8 | 27 |
| | | lectin receptor) [CD94] | | |
| B1 | H-U31116 | Human beta-sarcoglycan A3b | 35.09 | 33 |
| | | mRNA, complete cds | | |
| M297 C2 | H-U31278 | mitotic feedback control protein | 22.66 | 31.0 kDa |
| | | Madp2 homolog | | |
| M302 G2 | H-U31384 | guanine nucleotide-binding | 8.14 | 10 |
| | | protein, gamma 11 subunit | | |
| F4 | H-U31986 | Human cartilage-specific | 35.97 | 47 |
| | | homeodomain protein Cart-1 | | |
| | | mRNA, complete cds | | |
| M390 F3 | H-U32114 | caveolin 2 | 17.93 | 18.0 kDa |
| E4 | H-U32324 | Human interleukin-11 receptor | 46.53 | 54 |
| | | alpha chain mRNA, complete cds | | |
| F1 | H-U32576 | Apolipoprotein C-IV | 14.08 | 16 |
| M298 C4 | H-U32907 | p37NB protein | 34.54 | 39 |
| M300 D3 | H-U32944 | dynein, light chain 1, cytoplasmic | 9.9 | 15 |
| M297 D1 | H-U32989 | tryptophan 2,3-dioxygenase | 44.77 | 50.0 kDa |
| 166-51 | H-U33052 | “protein kinase PRK2 [human, | 110 | 108.3 |
| | | DX3 B-cell myeloma cell line, | | |
| | | mRNA]” | | |
| 166-64 | H-U33054 | “human G protein-coupled | 52 | 63.65 |
| | | receptor kinase GRK4 mRNA, | | |
| | | alpha splice variant” | | |
| 166-88 | H-U33055 | “human G protein-coupled | 60 | 60.1 |
| | | receptor kinase GRK4 mRNA, | | |
| | | beta splice variant” | | |
| 166-76 | H-U33056 | “human G protein-coupled | 58 | 58.59 |
| | | receptor kinase GRK4 mRNA, | | |
| | | gamma splice variant” | | |
| A2 | H-U34584 | | 17.71 | 31 |
| 169-87 | H-U34820 | human MAP kinase mRNA | 55 | 46.49 |
| 215-2 | H-U34822 | human JNK1 alpha2 protein | 55 | 47.04 |
| | | kinase (JNK1A2) mRNA | | |
| 169-37 | H-U35002 | human JNK2 betal protein kinase | 50 | 42.09 |
| | | (JNK2B1) mRNA | | |
| 169-25 | H-U35003 | human JNK2 beta2 protein kinase | 55 | 46.71 |
| | | (JNK2B2) mRNA | | |
| 167-16 | H-U35004 | human JNK1 betal protein kinase | 52 | 42.31 |
| | | (JNK1B1) mRNA | | |
| M300 B2 | H-U35048 | TSC-22 protein | 15.95 | 27 |
| M423 E5 | H-U35398 | Human G protein-coupled | 40.26 | 48.0 kDa |
| | | receptor mRNA, complete cds | | |
| A3 | H-U35735 | Human RACH1 (RACH1) | 42.9 | 78 |
| | | mRNA, complete cds | | |
| M250 E5 | H-U36764 | Eukaryotic translation initiation | 35.86 | 36.0 kDa |
| | | factor 3 (elF-3) p36 subunit, | | |
| | | transforming growth factor-beta | | |
| | | receptor II interacting protein 1 | | |
| M270 E4 | H-U37283 | microfibril-associated | 19.14 | 32 |
| | | glycoprotein-2 (GB: U37283) | | |
| M426 F3 | H-U37352 | Protein phosphatase 2A, | 56.65 | 55.0 kDa |
| | | regulatory subunit B′ alpha-1 | | |
| E1 | H-U37529 | Human substance P beta-PPT-A | 14.3 | 22 |
| | | mRNA, complete cds | | |
| M305 H5 | H-U37547 | apoptosis inhibitor | 68.09 | 64 |
| M424 D5 | H-U38480 | Human retinoid X receptor- | 51.04 | 61.0 kDa |
| | | gamma mRNA, complete cds | | |
| M270 F4 | H-U38810 | Human mab-21 cell fate- | | |
| | | determining protein homolog | | |
| | | (CAGR1) mRNA, | | |
| M467 F6 | H-U38904 | Human zinc finger protein C2H2- | 40.48 | 47.0 kDa |
| | | 25 mRNA, complete cds | | |
| E2 | H-U39318 | Human E2 ubiquitin conjugating | 16.28 | 22 |
| | | enzyme UbcH5C (UBCH5C) | | |
| | | mRNA, complete cds | | |
| 166-75 | H-U39657 | human MAP kinase kinase 6 | 40 | 36.81 |
| | | (MKK6) mRNA | | |
| M298 E4 | H-U39945 | human adenylate kinase 2 (adk2) | 26.3633 | 38.0 kDa |
| | | mRNA | | |
| 166-38 | H-U40282 | human integrin-linked kinase | 55 | 49.68 |
| | | (ILK) mRNA | | |
| 169-65 | H-U40343 | human CDK inhibitor p19INK4d | 18 | 18.33 |
| | | mRNA | | |
| E2 | H-U40705 | Homo sapiens telomeric repeat | 48.4 | 52 |
| | | binding factor (TRF1) mRNA, | | |
| | | complete cds | | |
| 166-50 | H-U40989 | human tat interactive protein | 60 | 53.09 |
| | | mRNA | | |
| M266 H6 | H-U41767 | metargidin precursor | 89.65 | 90 |
| M270 F3 | H-U41804 | Human putative T1/ST2 receptor | 25.08 | 35.0 kDa |
| | | binding protein precursor mRNA, | | |
| | | complete cds | | |
| D5 | H-U42360 | Human N33 gene | 38.28 | 38 |
| A1 | H-U43368 | Vascular endothelial growth | 22.88 | 33 |
| | | factor B | | |
| M421 G7 | H-U43901 | Human 37 kD laminin receptor | 32.56 | 58.0 kDa |
| | | precursor/p40 ribosome | | |
| | | associated protein gene, complete | | |
| | | cds | | |
| M392 C2 | H-U43923 | transcription factor SUPTH4 | 12.98 | 16.0 kDa |
| E2 | H-U46024 | Myotubular myopathy 1 | 66.44 | 58 |
| M330 A1 | H-U46838 | p105MCM | 90.42 | 97 |
| M476 E2 | H-U47677 | Human transcription factor E2F1 | 48.18 | 53.0 kDa |
| | | (E2F1) gene, promoter and | | |
| M421 H1 | H-U48707 | Human protein phosphatase-1 | 18.92 | 36.0 kDa |
| | | inhibitor mRNA, complete cds | | |
| M302 B7 | H-U49070 | peptidyl-prolyl isomerase PIN1 | 18.04 | 28.0 kDa |
| C1 | H-U49188 | Human placenta (Diff33) mRNA, | 54.45 | 70 |
| | | complete cds | | |
| M485 H2 | H-U49837 | Human LIM protein MLP mRNA, | 21.45 | 34.0 kDa |
| | | complete cds | | |
| D2 | H-U49897 | Homo sapiens phenylalanine | 49.83 | 64 |
| | | hydroxylase (PAH) mRNA, | | |
| | | complete cds | | |
| B2 | H-U49957 | Human LIM protein (LPP) | 67.43 | 67 |
| | | mRNA, partial cds | | |
| 166-16 | H-U50196 | human adenosine kinase mRNA | 50 | 38.02 |
| A4 | H-U50939 | Human amyloid precursor | 58.85 | 60 |
| | | protein-binding protein 1 mRNA, | | |
| | | complete cds | | |
| G3 | H-U51224 | Human U2AFBPL gene, complete | 52.8 | 55 |
| | | cds | | |
| M486 E3 | H-U51333 | Hexokinase 3 (white cell) | 101.64 | 100.0 kDa |
| M305 D1 | H-U51478 | ATPase, Na+/K+ transporting, | 30.8 | 36 |
| | | beta 3 subunit | | |
| M416 H3 | H-U52112 | Homo sapiens Xq28 genomic | 25.96 | 36.0 kDa |
| | | DNA in the region of the L1CAM | | |
| | | locus containing the genes for | | |
| | | neural cell adhesion molecule L1 | | |
| | | (L1CAM), arginine-vasopressin | | |
| | | receptor (AVPR2), C1 p115 (C1), | | |
| | | ARD1 N-acetyltransferase related | | |
| | | protein (TE2), renin-binding | | |
| | | protein (RbP), host cell factor 1 | | |
| | | (HCF1), and interleukin-1 | | |
| | | receptor-associated kinase | | |
| | | (IRAK) genes, complete cds, and | | |
| | | Xq28lu2 gene | | |
| M463 E1 | H-U53442 | human p38Beta MAP kinase | 40.99 | 49.0 kDa |
| | | mRNA | | |
| G3 | H-U53446 | Human mitogen-responsive | 84.81 | 98 |
| | | phosphoprotein DOC-2 mRNA, | | |
| | | complete cds | | |
| M463 C1 | H-U54617 | human pyruvate dehydrogenase | 45.28 | 52.0 kDa |
| | | kinase isoform 4 mRNA | | |
| 169-38 | H-U54645 | methylmalonyl-coA mutase | 38 | 25.59 |
| | | precursor | | |
| M300 H3 | H-U56255 | t-complex sterility protein | 12.54 | 16 |
| | | homolog CW-1 | | |
| C4 | H-U56417 | Human lysophosphatidic acid | 31.24 | 46 |
| | | acyltransferase-alpha mRNA, | | |
| | | complete cds | | |
| M305 A2 | H-U56637 | actin-capping protein alpha | 31.57 | 31 |
| | | subunit isoform 1 | | |
| M235 E6 | H-U56814 | Human DNase 1-Like III protein | 33.66 | 40.0 kDa |
| | | (DNAS1L3) mRNA, complete | | |
| | | cds, involved in apoptosis Binds | | |
| | | specifically to G-ACTIN AND | | |
| | | BLOCKS ACTIN | | |
| | | POLYMERIZATION. | | |
| D5 | H-U57059 | | 31.02 | 36 |
| B3 | H-U57093 | Human small GTP-binding | 24.09 | 34 |
| | | protein rab27b mRNA, complete | | |
| | | cds | | |
| D3 | H-U57099 | Human APEG-1 mRNA, | 12.54 | 20 |
| | | complete cds | | |
| F1 | H-U58331 | Sarcoglycan, delta (35 kD | 28.27 | 24 |
| | | dystrophin-associated | | |
| | | glycoprotein) | | |
| M512 F4 | H-U58334 | Human Bc12, p53 binding protein | 110.66 | 108.0 kDa |
| | | Bbp/53BP2 (BBP/53BP2) mRNA, | | |
| | | complete cds | | |
| B3 | H-U58516 | Human breast epithelial antigen | 42.68 | 50 |
| | | BA46 mRNA, complete cds | | |
| M250 E4 | H-U58522 | Human huntingtin interacting | 22.11 | 30 |
| | | protein (HIP2) mRNA, complete | | |
| | | cds | | |
| M419 G2 | H-U60207 | human stress responsive | 53.640 | 63.0 kDa |
| | | serine/threonine protein kinase | | |
| | | Krs-2 mRNA | | |
| M298 B2 | H-U60276 | arsA homolog (hASNA-I) | 36.63 | 47.0 kDa |
| B2 | H-U60521 | Human protease proMch6 (Mch6) | 45.87 | 52 |
| | | mRNA, complete cds | | |
| F3 | H-U61166 | Human SH3 domain-containing | 57.31 | 57 |
| | | protein SH3P17 mRNA, complete | | |
| | | cds | | |
| M250 B5 | H-U61232 | cofactor E (tubulin-folding | | |
| | | protein), REQUIRED FOR | | |
| | | VIABILITY IN THE ABSENCE | | |
| | | OF THE KINESIN-RELATED | | |
| | | CIN8 | | |
| A5 | H-U62392 | Homo sapiens zinc finger protein | 43.45 | 52 |
| | | mRNA, complete cds | | |
| G1 | H-U62801 | Human protease M mRNA, | 26.95 | 33 |
| | | complete cds | | |
| M266 B1 | H-U62962 | Int-6, Human Int-6 mRNA, | 49.06 | 52.0 kDa |
| | | complete cds | | |
| M300 G1 | H-U63295 | seven in absentia homolog | 31.13 | 36 |
| M306 H3 | H-U64198 | | 94.93 | 98 |
| H3 | H-U64863 | Human hPD-1 (hPD-1) mRNA, | 31.79 | 37 |
| | | complete cds | | |
| B3 | H-U65581 | Human ribosomal protein L3-like | 44.88 | 52 |
| | | mRNA, complete cds | | |
| M341 D1 | H-U65918 | DAZ homologue [DAZLA] | 32.56 | 36.0 kDa |
| M302 E1 | H-U65928 | Jun activation domain binding | 36.85 | 48.0 kDa |
| | | protein | | |
| M512 D3 | H-U66347 | Homo sapiens cAMP | 46.97 | 60.0 kDa |
| | | phosphodiesterase (PDE4C) | | |
| | | mRNA, 4C-426 isoform, | | |
| | | complete cds | | |
| M306 F3 | H-U66867 | ubiquitin-conjugating enzyme E21 | 17.49 | 28 |
| | | [UBE2I] | | |
| M416 E2 | H-U68111 | Human protein phosphatase | 22.66 | 37.0 kDa |
| | | inhibitor 2 (PPP1R2) gene | | |
| F2 | H-U68382 | Mannosidase, alpha B, lysosomal | 35.64 | 36 |
| G2 | H-U69141 | Glutaryl-Coenzyme A | 48.29 | 56 |
| | | dehydrogenase | | |
| B2 | H-U70660 | Human copper transport protein | 7.59 | 16 |
| | | HAH1 (HAH1) mRNA, complete | | |
| | | cds | | |
| M297 B2 | H-U71374 | peroxisomal membrane protein | 40.15 | 40.0 kDa |
| | | (Pex13p) | | |
| M306 A3 | H-U75272 | progastricsin [PGC] | 42.79 | 49.0 kDa |
| A2 | H-U75285 | Homo sapiens apoptosis inhibitor | 15.73 | 25 |
| | | survivin gene, complete cds | | |
| B2 | H-U77456 | Human nucleosome assembly | 41.36 | 50 |
| | | protein 2 mRNA, complete cds | | |
| C2 | H-U78294 | Homo sapiens 15S-lipoxygenase | 74.47 | 74 |
| | | mRNA, complete cds | | |
| F6 | H-U78302 | Human 2,4-dienoyl-CoA | 36.96 | 40 |
| | | reductase gene | | |
| M478 G3 | H-U78798 | Human TNF receptor associated | 57.53 | 65.0 kDa |
| | | factor 6 (TRAF6) mRNA, | | |
| | | complete cds | | |
| G3 | H-U80982 | Human myeloid-specific C/EBP- | 27.5 | 51 |
| | | epsilon transcription factor | | |
| | | (CEBPE) gene, complete cds | | |
| M468 B7 | H-U82256 | Homo sapiens arginase type II | 39.05 | 45.0 kDa |
| | | mRNA, complete cds | | |
| M465 B2 | H-U82812 | Human scavenger receptor | 38.28 | 48.0 kDa |
| | | cysteine rich Sp alpha mRNA, | | |
| | | complete cds | | |
| M484 D7 | H-U83410 | Human CUL-2 (cul-2) mRNA, | 82.06 | 85.0 kDa |
| | | complete cds | | |
| M467 E6 | H-U83460 | Human high-affinity copper | 21.01 | 32.0 kDa |
| | | uptake protein (hCTR1) mRNA, | | |
| | | complete cds | | |
| D2 | H-U84763 | Homo sapiens UCP3 mRNA, | 34.43 | 42 |
| | | complete cds | | |
| B2 | H-U86070 | Homo sapiens | 28.93 | 36 |
| | | phosphomannomutase mRNA, | | |
| | | complete cds | | |
| C2 | H-U90441 | Human prolyl 4-hydroxylase | 58.96 | 64 |
| | | alpha (II) subunit mRNA, | | |
| | | complete cds | | |
| B2 | H-U90543 | Human butyrophilin (BTF1) | 58.08 | 54 |
| | | mRNA, complete cds | | |
| H2 | H-U90545 | Human sodium phosphate | 44.22 | 36 |
| | | transporter (NPT4) mRNA, | | |
| | | complete cds | | |
| G2 | H-U90552 | Human butyrophilin (BTF5) | 56.54 | 48 |
| | | mRNA, complete cds | | |
| C3 | H-U91521 | Peroxisomal biogenesis factor 12 | 39.6 | 48 |
| H1 | H-U91641 | Human alpha2,8-sialyltransferase | 41.47 | 45 |
| | | mRNA, complete cds | | |
| C1 | H-U93869 | Human RNA polymerase III | 34.98 | 36 |
| | | subunit (RPC39) mRNA, | | |
| | | complete cds | | |
| F2 | H-U94346 | Human calpain-like protease | 70.4 | 65 |
| | | (htra-3) mRNA, complete cds | | |
| C2 | H-U94855 | Human translation initiation | 39.38 | 36 |
| | | factor 3.47 kDa subunit mRNA, | | |
| | | complete cds | | |
| M271 F7 | H-U95089 | Epidermal growth factor receptor. | 44.66 | 47 |
| M424 A5 | H-U95847 | Human GDNF receptor alpha | 50.71 | 52.0 kDa |
| | | mRNA, complete cds | | |
| D2 | H-U96094 | Human sarcolipin (SLN) mRNA, | 3.52 | 10 |
| | | complete cds | | |
| B3 | H-U96769 | Homo sapiens chondroadherin | 39.6 | 43 |
| | | gene, 5′flanking region and | | |
| M298 G2 | H-V00566 | prolactin | 25.08 | 35 |
| M298 H2 | H-V00571 | corticotropin-releasing factor | 21.67 | 49 |
| 217-61 | H-V00572 | phosphoglycerate kinase 1 | 50 | 45.94 |
| M314 B3 | H-V00597 | parathyroid hormone | 12.76 | 14 |
| M305 B8 | H-X00129 | retinol-binding protein 4, | 22 | 51 |
| | | interstitial [RBP4] | | |
| F2 | H-X00351 | Human mRNA for beta-actin | 41.36 | 41 |
| A4 | H-X00570 | apolipoprotein C-1 | 9.24 | 35 |
| M362 E1 | H-X01057 | interleukin 2 receptor, alpha | 30.03 | 40.0 kDa |
| | | [IL2RA] | | |
| A4 | H-X01677P | Human liver mRNA for | 10.45 | 10 |
| | | glyceraldehyde-3-phosphate | | |
| | | dehydrogenase (G3PD, EC | | |
| | | 1.2.1.12) | | |
| M271 D6 | H-X02152 | lactate dehydrogenase A [LDHA], | 36.63 | 45.0 kDa |
| | | L-LACTATE | | |
| | | DEHYDROGENASE M CHAIN | | |
| A1 | H-X02158 | Human gene for erythropoietin | 21.34 | 32 |
| H4 | H-X02415 | Human gene for fibrinogen | 48.18 | 50 |
| | | gamma chain | | |
| A5 | H-X02750 | Protein C (inactivator of | 50.82 | 53 |
| | | coagulation factors Va and VIIIa) | | |
| M302 B3 | H-X02751 | proto-oncogene N-ras | 20.9 | 25.0 kDa |
| D3 | H-X02812 | Human mRNA for transforming | 43.12 | 50 |
| | | growth factor-beta (TGF-beta) | | |
| M302 C1 | H-X03124 | tissue inhibitor of | 22.88 | 36.0 kDa |
| | | metalloproteinase 1 | | |
| M362 B1 | H-X03342 | ribosomal protein L32 | 14.96 | 24.0 kDa |
| M235 A2 | H-X03484 | human mRNA for raf oncogene | 71.350 | 73.0 kDa |
| M318 A3 | H-X03557 | interferon-induced protein 56 | 52.69 | 50.0 kDa |
| A3 | H-X03747 | ATPase, Na+/K+ transporting, | 33.44 | 45 |
| | | beta 1 polypeptide | | |
| M305 D2 | H-X04297 | ATPase, Na+/K+ transporting, | 112.64 | 99 |
| | | alpha subunit | | |
| M305 A5 | H-X04327 | 2,3-bisphosphoglycerate mutase | 28.6 | 36 |
| M271 G5 | H-X04588 | tropomyosin TM30nm, | 26.29 | 40.0 kDa |
| | | cytoskeletal | | |
| M305 C8 | H-X04741 | ubiquitin related protein | 23.43 | 28.0 kDa |
| M236 A5 | H-X05231 | matrix metalloproteinase 1 | 51.7 | 53.0 kDa |
| | | (interstitial collagenase) [MMPI], | | |
| | | CLEAVES COLLAGENS | | |
| 166-53 | H-X05246 | “phosphoglycerate kinase, testis | 50 | 45.94 |
| | | specific” | | |
| M236 A1 | H-X05908 | annexin 1, REGULATES | 38.17 | 40 |
| | | PHOSPHOLIPASE A2 | | |
| | | ACTIVITY, Binds CALCIUM | | |
| | | IONS | | |
| M250 A4 | H-X06234 | S100 calcium-binding protein A8 | 10.34 | 10.0 kDa |
| | | (calgranulin A) | | |
| M266 B6 | H-X06323 | ribosomal protein L3, isoform 1 | 38.39 | 39 |
| M313 A7 | H-X06617 | ribosomal protein S11 | 17.49 | 27 |
| M416 E4 | H-X06948 | High affinity IgE receptor alpha- | 28.38 | 36.0 kDa |
| | | subunit (FcERI) | | |
| M421 H7 | H-X07203 | Human mRNA for CD20 receptor | 32.78 | 40.0 kDa |
| | | (S7) | | |
| 217-2 | H-X07743 | pleckstrin | 38 | 38.57 |
| 217-73 | H-X07767 | “cAMP-dependent protein kinase, | 45 | 38.68 |
| | | alpha-catalytic subunit” | | |
| M305 B3 | H-X07898 | troponin C, skeletal, fast | 17.71 | 25 |
| M306 E1 | H-X07979 | integrin, beta 1 | 87.89 | 110 |
| A11 | H-X08004 | ras-related protein rap 1B | 20.24 | 38 |
| M235 A7 | H-X12387 | Cytochrome P450 IIIA3 | 55.44 | 60.0 kDa |
| | | (nifedipine oxidase chain 3) | | |
| M315 F1 | H-X12496 | glycophorin C | 14.19 | 24 |
| M316 D3 | H-X12517 | small nuclear ribonucleoprotein | 17.6 | 30.0 kDa |
| | | U1, C | | |
| M236 E5 | H-X12534 | guanine nucleotide-binding | 20.24 | 34.0 kDa |
| | | protein rap2, ras-oncogene related | | |
| M266 E3 | H-X12597 | High-mobility group (nonhistone | 23.76 | 37 |
| | | chromosomal) protein 1, placenta | | |
| 217-14 | H-X12656 | human mRNA for protein | 40 | 34.06 |
| | | phosphatase 2A (beta type) | | |
| H4 | H-X12662 | H. sapiens arginase gene exon 1 | 35.53 | 50 |
| | | and flanking regions (EC 3.5.3.1) | | |
| | | (and joined CDS) | | |
| C1 | H-X12953 | RAB2, member RAS oncogene | 23.43 | 29 |
| | | family | | |
| F5 | H-X13956 | Human 12S RNA induced by | 9.13 | 19 |
| | | poly(rI), poly(rC) and Newcastle | | |
| | | disease virus | | |
| M297 A1 | H-X15005 | laminin receptor 1 | 33.11 | 48.0 kDa |
| M315 E3 | H-X15088 | guanine nucleotide binding | 38.61 | 45 |
| | | protein (G protein), alpha | | |
| | | transducing (transducin) activity | | |
| | | polypeptide 1 [GNAT1] | | |
| G2 | H-X15183 | Human mRNA for 90-kDa heat- | 80.63 | 80 |
| | | shock protein | | |
| M385 C1 | H-X15422 | mannose-binding lectin, soluble | 27.39 | 27.0 kDa |
| | | (opsonic defect) [MBL] | | |
| M271 D7 | H-X15606 | INTERCELLULAR ADHESION | 30.36 | 37.0 kDa |
| | | MOLECULE-2 PRECURSOR | | |
| | | [Homo sapiens]. | | |
| M298 C5 | H-X15653 | uracil-DNA glycosylase | 33.55 | 37 |
| M302 B4 | H-X15822 | cytochrome-c oxidase, VIIa | 9.24 | 20 |
| | | subunit, liver | | |
| M305 A6 | H-X15940 | ribosomal protein L31 | 13.86 | 18 |
| M236 G5 | H-X15949 | interferon regulatory factor 2, | 38.5 | 54.0 kDa |
| | | BINDS AND REPRESSES | | |
| | | REGULATORY REGION OF | | |
| | | TYPE I IFN AND IFN- | | |
| | | INDUCIBLE MHC CLASS I | | |
| | | GENES. | | |
| M236 C2 | H-X16064 | translationally-controlled tumor | 19.03 | 35 |
| | | protein | | |
| M512 B5 | H-X16323 | Hepatocyte growth factor | 80.19 | 100.0 kDa |
| | | (hepapoietin A) | | |
| M315 C3 | H-X16461 | cell division cycle 2, G1 to S and | 32.78 | 40 |
| | | G2 to M [CDC2] | | |
| M297 G2 | H-X16832 | cathepsin H | 36.96 | 45.0 kDa |
| M271 B1 | H-X16983 | integrin, alpha 4 (CD49D, alpha 4 | 114.29 | 114 |
| | | subunit of VLA-4 receptor) | | |
| | | [ITGA4], IMPORTANT FOR | | |
| | | CELL-CELL ADHESION | | |
| | | FUNCTION. | | |
| M270 A7 | H-X17025 | plasminogen activator-inducible | 25.19 | 34 |
| | | c54, Human homolog of yeast IPP | | |
| | | isomerase | | |
| M302 C3 | H-X17042 | proteoglycan 1, secretory granule | 17.49 | 26 |
| B1 | H-X17206 | ribosomal protein S2 | 24.42 | 45 |
| B4 | H-X17254 | Transcription factor Eryfl | 45.54 | 53 |
| M311 H2 | H-X17610 | beta-1-glycoprotein, pregnancy- | 46.97 | 48.0 kDa |
| | | specific (GB: X17610) | | |
| M315 D1 | H-X17644 | G1 to S phase transition protein | 55 | 55 |
| | | (GST1) | | |
| M340 G1 | H-X51415 | lipase, hormone-sensitive [LIPE] | 84.59 | 98.0 kDa |
| M464 A7 | H-X51688 | Cyclin A | 47.63 | 47.0 kDa |
| M313 G1 | H-X51745 | major histocompatibility complex, | 40.26 | 50 |
| | | class I, A | | |
| M297 A2 | H-X51804 | putative receptor protein PMI | 21.23 | 30 |
| D4 | H-X51952 | Human UCP gene for uncoupling | 33.88 | 37 |
| | | protein exons 1 and 2 | | |
| M300 B1 | H-X52011 | muscle determining factor | 26.73 | 39 |
| M419 G1 | H-X52479 | “protein kinase c, alpha type” | 82.28 | 85.0 kDa |
| A2 | H-X52486 | Uracil-DNA glycosylase | 35.97 | 36 |
| E3 | H-X52520 | Tyrosine aminotransferase | 50.05 | 58 |
| B1 | H-X52638 | 6-phosphofructo-2- | 51.92 | 47 |
| | | kinase/fructose-2,6- | | |
| | | bisphosphatase | | |
| M509 C4 | H-X52730 | Human gene for | 31.13 | 35.0 kDa |
| | | phenylethanolamine N-methylase | | |
| | | (PNMT) (EC 2.1.1.28) | | |
| M235 C5 | H-X52839 | ribosomal protein L17 | 15.51 | 18 |
| M426 C2 | H-X52943 | Human mRNA for ATF-a | 53.24 | 64.0 kDa |
| | | transcription factor | | |
| M266 G5 | H-X53777 | ribosomal protein L23 | 20.35 | 31 |
| B4 | H-X53961 | Lactotransferrin | 78.32 | 78 |
| M462 C6 | H-X54150 | Fc fragment of IgA, receptor for | 31.68 | 37.0 kDa |
| M302 A6 | H-X54304 | myosin, light polypeptide 2, | 18.92 | 32.0 kDa |
| | | regulatory | | |
| M311 G2 | H-X54802 | cytochrome-c oxidase, IV subunit | 18.7 | 23.0 kDa |
| M270 H3 | H-X54871 | guanine nucleotide-binding | 23.76 | 33.0 kDa |
| | | protein Rab5B, ras-oncogene | | |
| | | related [RAB5B], PROTEIN | | |
| | | TRANSPORT. PROBABLY | | |
| | | INVOLVED IN VESICULAR | | |
| | | TRAFFIC (BYSIMILARITY). | | |
| M313 B6 | H-X54936 | placenta growth factor [PLGF*] | 16.5 | 22.0 kDa |
| M496 B2 | H-X55079 | Human lysosomal alpha- | 104.83 | 98.0 kDa |
| | | glucosidase gene exon 1 | | |
| D1 | H-X55330 | Aspartylglucosaminidase | 38.17 | 36 |
| E1 | H-X55448 | H. sapiens G6PD gene for | 25.41 | 30 |
| | | glucose-6-phosphate | | |
| | | dehydrogenase | | |
| M421 G6 | H-X56253 | Human MPR46 gene for 46 kd | 30.58 | 52.0 kDa |
| | | mannose 6-phosphate receptor | | |
| 169-89 | H-X56468 | 14-3-3 protein tau | 34 | 27.02 |
| M300 B4 | H-X56549 | fatty-acid-binding protein, muscle | 14.74 | 17 |
| M298 D2 | H-X56740 | guanine nucleotide-binding | 23.87 | 31.0 kDa |
| | | protein rab11 [RAB11*] | | |
| M266 E5 | H-X56932 | highly basic protein, 23 kDa | 22.44 | 30.0 kDa |
| M318 G1 | H-X57025 | insulin-like growth factor I | 16.94 | 18 |
| M305 F5 | H-X57348 | protein kinase C inhibitor | 27.39 | 35.0 kDa |
| M236 D6 | H-X57351 | interferon-induced protein 1-8D | 14.63 | 24 |
| H3 | H-X57352 | interferon-induced protein 1-8U | 14.74 | 38 |
| M305 B6 | H-X58079 | S-100 protein, alpha chain | 10.45 | 11 |
| E6 | H-X59131 | H. sapiens D13S106 mRNA for a | 34.76 | 50 |
| | | highly charged amino acid | | |
| | | sequene | | |
| M248 H5 | H-X59268 | transcription factor IIB [TCF2B*] | 34.87 | 49 |
| E2 | H-X59357 | Epstein-Barr virus small RNA- | 14.19 | 36 |
| | | associated protein | | |
| M236 D4 | H-X59417 | macropain, iota subunit, THE | 27.17 | 36 |
| | | INTERACTION OF CALPONIN | | |
| | | WITH ACTIN INHIBITS | | |
| | | ACTOMYOSIN MG-ATPASE | | |
| | | ACTIVITY | | |
| M271 H4 | H-X59618 | ribonucleotide reductase, small | 42.9 | 46 |
| | | subunit | | |
| M250 G3 | H-X59710 | CAAT-box DNA-binding protein, | 22.66 | 34 |
| | | subunit B, CCAAT-BINDING | | |
| | | TRANSCRIPTION FACTOR | | |
| | | SUBUNIT A [Homo sapiens] | | |
| M423 E2 | H-X59711 | Nuclear transcription factor Y, | 38.28 | 48.0 kDa |
| | | alpha | | |
| M271 C7 | H-X59798 | Cyclin D1 (PRAD1; parathyroid | 32.56 | 40.0 kDa |
| | | adenomatosis 1). ESSENTIAL | | |
| | | FOR THE CONTROL OF THE | | |
| | | CELL CYCLE AT THE G1/S | | |
| | | (START) TRANSITION. | | |
| M270 H5 | H-X59834 | calmodulin | 41.14 | 53.0 kDa |
| M416 D5 | H-X59871 | Transcription factor 7 (T-cell | 29.59 | 36.0 kDa |
| | | specific) | | |
| M485 D6 | H-X60036 | Phosphate carrier, mitochondrial | 39.82 | 37.0 kDa |
| M250 D4 | H-X60489 | translation elongation factor 1, | 24.86 | 33.0 kDa |
| | | beta | | |
| F5 | H-X60592 | Human CDw40 mRNA for nerve | 30.58 | 46 |
| | | growth factor receptor-related B- | | |
| | | lymphocyte activation molecule | | |
| M312 F3 | H-X61587 | ras-related rhoG | 21.12 | 21.0 kDa |
| F9 | H-X61622 | cyclin-dependent kinase 2 | 32.89 | 56 |
| | | [CDK2] | | |
| M313 E3 | H-X61970 | macropain, zeta subunit | 26.62 | 35.0 kDa |
| M428 D1 | H-X62055 | tyrosine phosphatase, non- | 65.78 | 66.0 kDa |
| | | receptor type 6 | | |
| M248 C4 | H-X62534 | high mobility group protein 2, | 23.1 | 37 |
| | | BINDS PREFERENTIALLY | | |
| | | SINGLE-STRANDED DNA | | |
| | | AND UNWINDS DOUBLE | | |
| | | STRANDED DNA. | | |
| M305 F3 | H-X62753 | folate-binding protein | 28.38 | 36 |
| M476 G2 | H-X63468 | H. sapiens mRNA for transcription | 48.4 | 53.0 kDa |
| | | factor TFIIE alpha | | |
| G6 | H-X63469 | General transcription factor TFIIE | 32.12 | 56 |
| | | beta subunit, 34 kD | | |
| G4 | H-X63522 | H. sapiens mRNA DAUD16 for | 58.74 | 54 |
| | | retinoic acid X receptor b | | |
| M316 G2 | H-X63526 | translation elongation factor 1, | 48.18 | 52.0 kDa |
| | | gamma | | |
| M305 C5 | H-X63527 | ribosomal protein L19 | 21.67 | 33 |
| E2 | H-X63629 | Cadherin 3 (P-cadherin) | 91.3 | 110 |
| D4 | H-X64037-2 | General transcription factor IIF, | 56.98 | 64 |
| | | polypeptide 1 (74 kD subunit) | | |
| M302 C6 | H-X64559 | tetranectin | 22.33 | 32.0 kDa |
| M271 H1 | H-X64728 | choroideremia-like [CHML], | 72.27 | 98 |
| | | H. sapiens CHML mRNA | | |
| M270 E1 | H-X64810 | proprotein convertase | 82.94 | 90 |
| | | subtilisin/kexin type I [PCSK1], | | |
| | | INVOLVED IN PROCESSING | | |
| | | OF HORMONE AND OTHER | | |
| | | PROTEIN PRECURSORS | | |
| M311 F4 | H-X64877 | complement factor H-related | 29.81 | 36.0 kDa |
| | | protein | | |
| M388 D1 | H-X65293 | protein kinase C, epsilon | 81.18 | 96.0 kDa |
| | | [PRKCE] | | |
| B5 | H-X65873 | kinesin, heavy polypeptide | 106.04 | 34 |
| F4 | H-X66079 | Spi-B transcription factor (Spi- | 28.93 | 54 |
| | | 1/PU.1 related) | | |
| F3 | H-X66114 | 2-oxoglutarate carrier protein | 0 | 37 |
| | | [OGMT*] | | |
| M305 C6 | H-X66141 | myosin, light polypeptide 2, | 18.37 | 31 |
| | | regulatory, ventricular | | |
| M419 H1 | H-X66357 | cell division protein kinase 3 | 33.620 | 44.0 kDa |
| 166-13 | H-X66358 | serine/threonine-protein kinase | 45 | 39.45 |
| | | KKIALRE | | |
| 166-25 | H-X66360 | serine/threonine-protein kinase | 60 | 57.60 |
| | | PCTAIRE-2 | | |
| M419 A2 | H-X66363 | serine/threonine-protein kinase | 54.600 | 64.0 kDa |
| | | PCTAIRE-1 | | |
| 166-37 | H-X66364 | H. sapiens mRNA PSSALRE for | 38 | 32.19 |
| | | serine/threonine protein kinase | | |
| M419 B2 | H-X66365 | cell division protein kinase 6 | 35.900 | 46.0 kDa |
| H3 | H-X66839 | H. sapiens MaTu MN mRNA for | 50.6 | 54 |
| | | p54/58N protein | | |
| M266 G3 | H-X67325 | interferon, alpha-inducible gene | 13.53 | 13 |
| | | p27 | | |
| M462 H7 | H-X67594 | Melanocortin 1 receptor (alpha | 34.98 | 44.0 kDa |
| | | melanocyte stimulating hormone | | |
| | | receptor) | | |
| M236 C5 | H-X67951 | Proliferation-associated gene A | 22 | 34 |
| | | (natural killer-enhancing factor | | |
| | | A), PAGA | | |
| H3 | H-X68486 | Adenosine receptor A2 | 45.43 | 45 |
| M429 E3 | H-X68561 | Sp4 transcription factor | 86.35 | 86.0 kDa |
| M430 F2 | H-X69151 | ATP synthase, H+ transporting, | 42.13 | 58.0 kDa |
| | | subunit C, vacuolar | | |
| M236 C3 | H-X69392 | ribosomal protein L26 | 16.06 | 29 |
| B3 | H-X69532 | H. sapiens gene for inter-alpha- | 100.32 | 98 |
| | | trypsin inhibitor heavy chain H1, | | |
| | | exons 1-3 | | |
| M236 F5 | H-X69654 | ribosomal protein S26 | 12.76 | 18 |
| M421 C8 | H-X70218 | Protein phosphatase 4 (formerly | 33.88 | |
| | | X), catalytic subunit | | |
| M266 H5 | H-X70848 | protein phosphatase 1, alpha | 36.41 | 37 |
| | | catalytic subunit | | |
| E1 | H-X70940 | Eukaryotic translation elongation | 51.04 | 60 |
| | | factor 1 alpha 2 | | |
| M270 F1 | H-X72215 | [PITI], POU domain, class 1, | 32.12 | 40.0 kDa |
| | | transcription factor 1 (Pit1, | | |
| | | growth hormone factor 1) | | |
| M271 A7 | H-X72760 | Laminin, beta 2 (laminin S), S- | 67.87 | 75.0 kDa |
| | | LAMININ IS A LAMININ-LIKE | | |
| | | ADHESIVE PROTEIN | | |
| | | CONCENTRATED IN THE | | |
| | | SYNAPTIC CLEFT OF THE | | |
| | | NEUROMUSCULAR | | |
| | | JUNCTION. | | |
| M235 B1 | H-X72841 | Human retinoblastoma-binding | 46.86 | 52.0 kDa |
| | | protein (RbAp46) mRNA, | | |
| | | complete cds, IEF 7442 | | |
| | | (GB: X72841) | | |
| 217-25 | H-X73428 | DNA-binding protein inhibitor | 20 | 17.08 |
| | | ID-3 | | |
| M305 B5 | H-X73459 | signal recognition particle, | 15.07 | 20 |
| | | subunit 14 | | |
| M250 D6 | H-X73460 | ribosomal protein L3, isoform 2, | 44.44 | 50.0 kDa |
| | | COMPONENT OF THE LARGE | | |
| | | SUBUNIT OF CYTOPLASMIC | | |
| | | RIBOSOMES | | |
| M462 D8 | H-X74008 | Protein phosphatase 1, catalytic | 35.64 | 46.0 kDa |
| | | subunit, gamma isoform | | |
| M266 G2 | H-X74104 | Signal sequence receptor, beta; | 20.24 | 27 |
| | | translocon-associated protein, | | |
| | | beta subunit | | |
| M266 E7 | H-X74262 | retinoblastoma binding protein | 46.86 | 50.0 kDa |
| | | RbAp48 | | |
| H1 | H-X74330 | DNA primase polypeptide 1 | 46.31 | 51 |
| | | (49 kD) | | |
| M313 F3 | H-X74570 | gal beta (1-3/1-4) GIcNAc alpha- | 36.3 | 46.0 kDa |
| | | 2,3 sialyltransferase (GB: X74570) | | |
| M429 H3 | H-X74764 | H. sapiens mRNA for receptor | 94.120 | 98.0 kDa |
| | | protein tyrosine kinase | | |
| M271 E6 | H-X75042 | V-rel avian reticuloendotheliosis | 68.2 | 88 |
| | | viral oncogene homolog | | |
| M305 G2 | H-X75252 | phosphatidylethanolaminc- | 20.68 | 30 |
| | | binding protein | | |
| M302 G1 | H-X75593 | guanine nucleotide-binding | 22.44 | 32.0 kDa |
| | | protein rab13 | | |
| 166-49 | H-X75958 | H. sapiens trkB mRNA for | 55 | 52.54 |
| | | protein-tyrosine kinase | | |
| C4 | H-X76013 | H. sapiens QRSHs mRNA for | 85.36 | 85 |
| | | glutaminyl-tRNA synthetase | | |
| A2 | H-X76029 | H. sapiens mRNA for | 19.25 | 20 |
| | | neuromedin U | | |
| M305 D5 | H-X76228 | ATP synthase, H+ transporting, | 24.97 | 36 |
| | | subunit E, vacuolar | | |
| M298 F6 | H-X76648 | glutaredoxin | 11.77 | 11.0 kDa |
| M311 A4 | H-X76717 | metallothionein II | 6.82 | 14 |
| C4 | H-X77533 | H. sapiens mRNA for activin type | 56.43 | 61 |
| | | II receptor | | |
| H2 | H-X77548 | H. sapiens cDNA for RFG | 67.65 | 67 |
| 169-41 | H-X77743 | H. sapiens CDK activating kinase | 45 | 38.13 |
| | | mRNA | | |
| A4 | H-X77909 | H. sapiens IKBL mRNA | 42.02 | 52 |
| M305 CI | H-X78136 | heterogeneous nuclear | 40.26 | 40.0 kDa |
| | | ribonucleoprotein E2 | | |
| M306 G2 | H-X78416 | casein, alpha [CSN1] | 20.46 | 33 |
| M271 C2 | H-X78678 | ketohexokinase (fructokinase) | 32.89 | 39 |
| | | [KHK], H. sapiens KHK mRNA | | |
| | | for ketohexokinase, clone | | |
| | | pHKHK3a | | |
| M305 D4 | H-X79193 | cyclin-dependent kinase 7 | 38.17 | 35 |
| | | (homolog of Xenopus MO15 cdk- | | |
| | | activating kinase) [CDK7] | | |
| M431 F2 | H-X79389 | glutathione S-transferase T1 | 26.51 | 34.0 kDa |
| M298 C6 | H-X79537 | glycogenin | 30.8 | 34.0 kDa |
| M440 C1 | H-X79865 | H. sapiens Mrp17 mRNA | 21.89 | 31.0 kDa |
| M298 F5 | H-X80229 | protein kinase PKN | 52.8 | 64.0 kDa |
| 167-39 | H-X80230 | H. sapiens mRNA (clone C-2k) | 42 | 40.99 |
| | | mRNA for serine/threonine | | |
| | | protein kinase | | |
| 217-49 | H-X80343 | H. sapiens p35 mRNA for | 40 | 33.84 |
| | | regulatory subunit of cdk5 kinase | | |
| M270 D7 | H-X80695 | cytochrome oxidase-assembly | 47.96 | 50 |
| | | protein, OXA1, H. sapiens | | |
| | | OXA1Hs mRNA | | |
| M266 B5 | H-X80909 | nascent polypeptide-associate | 23.76 | 37.0 kDa |
| | | complex, alpha | | |
| M416 D9 | H-X80910 | Protein phosphatase 1, catalytic | 36.08 | 45.0 kDa |
| | | subunit, beta isoform | | |
| E2 | H-X81198 | Archain | 52.03 | 63 |
| 169-6 | H-X81817 | H. sapiens BAP31 mRNA | 32 | 27.13 |
| E4 | H-X82018 | H. sapiens mRNA for ZID protein | 46.75 | 57 |
| M313 D7 | H-X82456 | MLN50 | 28.82 | 33 |
| A2 | H-X82629 | H. sapiens mRNA for Mox-2 | 33.44 | 42 |
| M236 D1 | H-X83006 | lipocalin, neutrophil gelatinase | 21.89 | 34.0 kDa |
| | | associated | | |
| 166-40 | H-X83107 | H. sapiens Bmx mRNA for | 75 | 74.32 |
| | | cytoplasmic tyrosine kinase | | |
| E3 | H-X83425 | H. sapiens LU gene for Lutheran | 69.19 | 59 |
| | | blood group glycoprotein | | |
| C6 | H-X83703 | H. sapiens mRNA for cytokine | 35.2 | 54 |
| | | inducible nuclear protein | | |
| M416 H2 | H-X83928 | H. sapiens mRNA for transcription | 23.32 | 33.0 kDa |
| | | factor TFIID subunit TAFII28 | | |
| 166-17 | H-X85106 | H. sapiens mRNA for ribosomal | 90 | 80.70 |
| | | S6 kinase | | |
| 166-39 | H-X85337 | H. sapiens mRNA for myosin light | 110 | 109.0 |
| | | chain kinase | | |
| D2 | H-X85750 | H. sapiens mRNA for transcript | 26.29 | 30 |
| | | associated with monocyte to | | |
| | | macrophage differentiation | | |
| M266 E6 | H-X87176 | 17-beta-hydroxysteroid | 81.07 | 65 |
| | | dehydrogenase, type 4 | | |
| M297 F2 | H-X87689 | CLCP | 23.21 | 33.0 kDa |
| M300 A2 | H-X87843 | cyclin H assembly factor | 34.1 | 47 |
| M271 E3 | H-X89750 | homeotic protein, TGIF, | 30.03 | 32.0 kDa |
| | | H. sapiens mRNA for TGIF | | |
| | | protein | | |
| M235 G1 | H-X90529 | guanine nucleotide-binding | 34.54 | 40 |
| | | protein ragA [RAGA] | | |
| M302 E6 | H-X90583 | translocon-associated protein, | 19.14 | 28.0 kDa |
| | | delta | | |
| M306 G1 | H-X90872 | gp2512 | 23.65 | 33 |
| M416 D2 | H-X91504 | Transcription factor COUP 2 | 22.22 | 32.0 kDa |
| | | (a.k.a. ARP1) | | |
| M250 B3 | H-X92098 | transmembrane protein rnp24 | 22.22 | 30 |
| M271 G7 | H-X92106 | bleomycin hydrolase | 50.16 | 55.0 kDa |
| | | PROTECTING NORMAL AND | | |
| | | MALIGNANT CELLS FROM | | |
| | | BLM TOXICITY. | | |
| F3 | H-X92715 | Zinc finger protein 74 (Cos52) | 63.03 | 47 |
| M270 H6 | H-X92720 | H. sapiens mRNA for | 70.51 | 71 |
| | | phosphoenolpyruvate | | |
| | | carboxykinase | | |
| H5 | H-X92762 | H. sapiens mRNA for tafazzins | 32.23 | 37 |
| | | protein | | |
| M298 D3 | H-X93036 | MAT-8 | 9.68 | 16.0 kDa |
| M476 A5 | H-X93595 | H. sapiens mRNA for NK receptor | 50.16 | 56.0 kDa |
| | | (clone 17.1C) | | |
| M417 D2 | H-X93920 | protein tyrosine phosphatase | 41.980 | 48.0 kDa |
| | | foreskin | | |
| A5 | H-X95592 | H. sapiens mRNA for C1D protein | 15.62 | 28 |
| M298 B4 | H-X95648 | translation initiation factor 2B, | 33.66 | 34.0 kDa |
| | | alpha subunit | | |
| F3 | H-X95735 | H. sapiens mRNA for zyxin 2 | 63.03 | 72 |
| M386 B1 | H-X96752 | L-3-hydroxyacyl-CoA | 34.65 | 45.0 kDa |
| | | dehydrogenase, SCHAD gene | | |
| M422 B6 | H-X97229 | H. sapiens mRNA for NK | 41.58 | 48.0 kDa |
| | | receptor, clone library 15.212 | | |
| B3 | H-X98173 | H. sapiens mRNA for MACH- | 51.15 | 51 |
| | | alpha-2 protein | | |
| 166-14 | H-X99325 | H. sapiens mRNA for Ste20-like | 55 | 46.93 |
| | | kinase | | |
| C4 | H-X99459 | H. sapiens mRNA for sigma 3B | 21.34 | 30 |
| | | protein | | |
| M424 C4 | H-Y00291 | Human hap mRNA encoding a | 49.39 | 59.0 kDa |
| | | DNA-binding hormone receptor | | |
| M386 H1 | H-Y00345 | polyadenylate-binding protein | 69.74 | 70.0 kDa |
| M469 A2 | H-Y00630 | Plasminogen activator inhibitor, | 45.76 | 46.0 kDa |
| | | type II (arginine-serpin) | | |
| M305 E1 | H-Y00711 | lactate dehydrogenase B | 36.85 | 38.0 kDa |
| H2 | H-Y00764 | ubiquinol/cytochrome c reductase | 10.12 | 33 |
| | | hinge protein | | |
| F5 | H-Y07848 | H. sapiens EWS, gar22, rrp22 and | 36.3 | 50 |
| | | bam22 genes | | |
| M305 G6 | H-Z11559 | iron-responsive element binding | 97.9 | 98 |
| | | protein 1 [IREB1] | | |
| M250 F3 | H-Z11566 | Pr22 protein, STATHMIN | 16.5 | 22.0 kDa |
| | | [Homo sapiens], SERVES AS | | |
| | | RELAY (VIA | | |
| | | PHOSPHORYLATION) FOR | | |
| | | DIVERSE SECOND | | |
| | | MESSENGER PATHWAYS | | |
| 169-73 | H-Z11695 | H. sapiens 40 kDa protein kinase | 50 | 38.35 |
| | | related to rat ERK2 | | |
| M475 C8 | H-Z11737 | Flavin-containing | 61.49 | 70.0 kDa |
| | | monooxygenase 4 | | |
| C1 | H-Z11898 | Octamer binding protein 3 | 39.71 | 50 |
| M266 H4 | H-Z12830 | SSR, alpha subunit | 31.57 | 42.0 kDa |
| A3 | H-Z14000 | Ring finger protein 1 | 41.58 | 50 |
| M300 E1 | H-Z14978 | actin-related protein | 41.47 | 49 |
| G1 | H-Z19002 | H. sapiens of PLZF gene encoding | 74.14 | 84 |
| | | kruppel-like zinc finger protein | | |
| H1 | H-Z21966 | POU homeobox protein | 33.22 | 43 |
| M248 G3 | H-Z23139 | CLASS II | 29.04 | 34 |
| | | HISTOCOMPATIBILITY | | |
| | | ANTIGEN, M BETA CHAIN | | |
| | | PRECURSOR [Homo sapiens] | | |
| D3 | H-Z26876 | ribosomal protein L38 | 7.81 | 35 |
| F2 | H-Z28339 | H. sapiens mRNA for delta 4-3- | 35.97 | 43 |
| | | oxosteroid 5 beta-reductase | | |
| M298 B3 | H-Z28407 | ribosomal protein L8 | 28.38 | 39.0 kDa |
| M313 C3 | H-Z29330 | ubiquitin-conjugating enzyme | 20.24 | 34 |
| | | UbcH2, 23 kDa | | |
| M271 F3 | H-Z29677 | guanine nucleotide-binding | 20.35 | 28.0 kDa |
| | | protein, ras-related | | |
| M465 C2 | H-Z30425 | H. sapiens mRNA for orphan | 38.39 | 34.0 kDa |
| | | nuclear hormone receptor | | |
| M302 F5 | H-Z31357 | cysteine dioxygenase | 22.11 | 31.0 kDa |
| M340 C1 | H-Z31695 | inositol polyphosphate 5- | 40.04 | 49.0 kDa |
| | | phosphatase, 43 kDa | | |
| E3 | H-Z32564-2 | H. sapiens FRGAMMA mRNA | 26.84 | 36 |
| | | (819bp) for folate receptor | | |
| M236 H1 | H-Z35227 | small G protein, TTF, RAS- | 21.12 | 30.0 kDa |
| | | RELATED PROTEIN RAC1 | | |
| A10 | H-Z35491 | H. sapiens mRNA for novel | 30.25 | 60 |
| | | glucocorticoid receptor-associated | | |
| | | protein | | |
| M440 G5 | H-Z37986 | H. sapiens mRNA for | 25.41 | 28.0 kDa |
| | | phenylalkylamine binding protein | | |
| M297 E2 | H-Z47087 | cyclin A/cyclin-dependent kinase | 18.04 | 30.0 kDa |
| | | 2-associated p19 | | |
| F1 | H-Z48051 | H. sapiens gene for myelin | 27.28 | 31 |
| | | oligodendrocyte glycoprotein | | |
| | | (MOG) | | |
| A2 | H-Z48475 | Glucokinase regulator | 68.86 | 70 |
| M302 E4 | H-Z48570 | sperm zona pellucida-binding | 16.72 | 24 |
| | | protein | | |
| M266 A2 | H-Z68907 | Human clone ID 193225 NAD | 43.34 | 45.0 kDa |
| | | (H)-specific isocitrate | | |
| | | dehydrogenase gamma subunit | | |
| | | mRNA, alternatively spliced, | | |
| | | partial cds | | |
| G1 | H-Z83850 | Human DNA sequence from PAC | 45.76 | 60 |
| | | 82J11 and cosmid U134E6 on | | |
| | | chromosome Xq22. Contains NIK | | |
| | | like and Thyroxin-binding | | |
| | | globulin precursor (T4-binding | | |
| | | globulin, TBG) genes, ESTs and | | |
| | | STSs | | |
| H4 | H-Z97171 | Homo sapiens GLC1A (trabecular | 55.55 | 55 |
| | | meshwork induced glucocortcoid | | |
| | | response) gene, exon 1, joined | | |
| | | CDS | | |
| M421 D5 | H-Z97632 | Human DNA sequence from PAC | 28.49 | 38.0 kDa |
| | | 196E23 on chromosome | | |
| | | Xq26.1-27.2. | | |
| | | Contains the TAT-SF1 | | |
| | | (HIV-1 transcriptional elongation | | |
| | | factor TAT cofactor TAT-SF1) | | |
| | | gene, the BRS3 (Bombesin | | |
| | | Receptor subtype-3 (Uterine | | |
| | | Bombesin Receptor, BRS-3) | | |
| | | gene, an unknown gene coding | | |
| | | for two isoforms, a predicted CpG | | |
| | | island, ESTs and STSs | | |

### EXAMPLE 3

### Construction of Expression Plasmids

The following example illustrates the construction of the expression vectors used in the Examples above. Similar modifications can be made in other vectors for use in creating libraries of expressible gene sequences.

The vector pcDNA3.1/V5-His was obtained from Invitrogen (cat #V810-20) and modified slightly so that it carried an gene sequence for Zeocin™ resistance and lacked the multiple cloning site. A 100μg aliquot was suspended in 200 μl medical irrigation (MI) water. A 5 μl aliquot was saved for gel analysis. The remainder was transferred to a 1.7 ml Eppendorf tube. The vector was digested with HindIII (400 U) using Promega Buffer E (final volume=400 μl). The reaction ran 3 hours at 37° C. An aliquot was checked for completeness of digestion by running on an 0.8% agarose gel in 1X TAE, and visualizing with ethidium bromide.

The digested vector was treated with 200 μl phenol/chloroform (pH7.5) according to standard procedures, and the DNA precipitated from the aqueous phase using 1/10 volume 3M NaOAc and 2 volumes 100% EtOH at room temperature, followed by washing with 80% EtOH. The pellet was resuspended in 100 μl MI water.

Two oligonucleotides were added to the resuspended DNA (Topo -H (40 μg) 5′-(P)AGCTCGCCCTTATTCCGATAGTG (SEQ ID NO:3), Topo-4 (12 μg) 5′-(P)AGGGCG (SEQ ID NO:4)), plus 17 μl 10X Promega T4 Ligase buffer. The tube was placed on ice and the volume increased to 170 μl with MI water. The oligos were ligated to the vector using 20U Promega T4 DNA ligase, incubated at 12° C. overnight.

The vector was treated with 100 μl phenol/chloroform and the aqueous phase precipitated as described above. The pelleted DNA was resuspended in 150 μl of sterile water the redigested with HindIII (17 μl Promega Buffer E, 200 U HindIII- 37° C., 1 hour). The redigested DNA was re-extracted with phenol/chloroform and precipitated with 1/10 volume 3M NaOAc and 7/10 volume isopropanol, then washed with 80% EtOH.

The pelleted DNA was resuspended in 82 μl TE buffer (10 mM Tris, pH8.0, 1 mM EDTA, pH 8.0). A 2 μl aliquot was used to check the foregoing procedure using agarose gel electrophoresis as described above. The remaining 80 μl was transferred to a Falcon tube and mixed with 16 μg Topo-5 oligonucleotide (5′-(P)CAACACTATCGGAATA (SEQ ID NO:5). To this mixture was added 190 μl NEB Restriction Buffer #1 (room temperature). The total reaction mixture was adjusted to 1.9 mls with MI water. Vaccinia Topoisomerase I enzyme was added (80 μg) and the reaction tube placed in a 37° C. water bath for 15 minutes.

After 15 minutes, 200 μl of room temperature Topo-10X stop buffer was added (100 mM Tris 7.4, 110 mM EDTA, bromophenol blue). The entire volume was loaded onto an agarose gel (1.2 gr agarose/130 mls 1X TAE) and run at 70 volts until the bromophenol blue dye had run down about ½ in (volume in the loading well was kept constant by the addition of 1X TE). The voltage was reversed for 90 seconds. The contents of the loading well were transferred to a 15 ml Falcon tube and placed on ice. 2 mls of cold Topo-2X Wash Buffer (60 mM Tris 7.4, 1 mM EDTA, 4 mM dithiothreitol (DTT), 200 μg/ml bovine serum albumin (BSA)) was added and the volume then adjusted to 4 mls with cold Topo-1X Enzyme Dilution Buffer (50% glycerol, 50 mM Tris 7.4, 1 mM EDTA, 2 mM DTT, 0.1% Triton X-100, 100 μg/ml BSA) plus 4 mls Topo-Glycerol mix (90% glycerol, 10% 50 mM TE pH 7.4, 0.1% Triton X-100) and stored until needed.

A similar procedure was used to make Topo-adapted pYES2 (Invitrogen cat #V825-20).

While the foregoing has been presented with reference to particular embodiments of the invention, it will be appreciated by those skilled in the art that changes in these embodiments may be made without departing from the principles and spirit of the invention, the scope of which is defined by the appended claims.

That which is claimed is:

1. A method for producing a library of expressible coding regions comprising the steps of:
(a) amplifying a plurality of coding regions using at least one coding region specific primer,
(b) inserting each coding region into an expression vector, and
(c) verifying the size and orientation of the inserted coding region.

2. The method according to claim 1 further comprising transforming cells with the vector containing the verified coding region.

3. The method according to claim 1 further comprising purifying the amplified coding region prior to insertion into an expression vector.

4. The method according to claim 1 wherein the coding regions encode full-length proteins.

5. The method according to claim 4 wherein the 5′ primer used for amplification starts with the nucleotides CACCATFG and the 3′ primer causes the amplification product to end at the third position of the codon immediately preceding the stop codon of the coding region being amplified plus a single adenine residue.

6. The method according to claim 3 wherein the purification is performed using agarose gel electrophoresis.

7. The method according to claim 6 wherein the agarose is low melt agarose.

8. The method according to claim I wherein insertion of the amplified coding region into an expression vector is performed using an enzyme that both cleaves and ligates DNA.

9. The method according to claim 3 wherein the purification is performed using low melt agarose gel electrophoresis and insertion of the amplified coding region into an expression vector is performed using an enzyme that both cleaves and ligates DNA.

10. The method according to claim 8 wherein said enzyme is a type I topoisomerase or a site-specific recombinase.

11. The method according to claim 10 wherein said enzyme is vaccinia DNA topoisomerase, lambda integrase, FLP recombinase or P1-Cre protein.

12. A method according to claim 11 wherein said enzyme is vaccinia DNA topoisomerase.

13. The method of claim 1 wherein the expression vector is a eukaryotic expression vector.

14. The method of claim 13 wherein said eukaryotic expression vector is pYES2/GS or pcDNA3.1/GS.

15. The method of claim 1 wherein the expression vector is a prokaryotic expression vector.

16. The method of claim 15 wherein said prokaryotic expression vector is pBAD.

17. The method according to claim 1 wherein the expression vector comprises one or more elements selected from: a promoter-enhancer sequence, a selection marker sequence, an origin of replication, an affinity purification tag sequence, an inducible element sequence and an epitope-tag sequence.

18. The method of claim 1 wherein size and orientation of the insert is verified using a polymerase chain reaction protocol.

19. The method of claim 18 wherein said verification is performed using whole cell lysates.

20. The method of claim 1 wherein the coding regions to be amplified are open reading frame sequences in prokaryotic DNA or eukaryotic DNA.

21. The method according to claim 20 wherein the eukaryotic DNA is obtained from yeast or mammalian cells.

22. The method according to claim 1 wherein the coding regions being amplified encode members of a family of proteins.

23. The method according to claim 22 wherein the proteins are human proteins.

24. The method according to claim 23 wherein the family of proteins are kinases, phosphatases, transcription factors, oncogenes, or tumor suppressors.

25. The method according to claim 1 wherein steps (a) and (b) are performed in a multiwell microtiter plate.

26. The method according to claim 1 wherein coding regions of the correct size and in the correct orientation are roboticly selected for transformation into cells for expression.

27. The method according to claim 2 comprising the additional step of verifying that the transformed cells express the coding region.

28. The method according to claim 2 wherein the transformed cells are eukaryotic cells or prokaryotic cells.

29. A method according to claim 28 wherein the eukaryotic cells are CHO cells or S. cerevisiea cells.

30. An expression library of coding regions produced according to the method of claim 1.

31. The library according to claim 30 wherein the coding regions encode yeast proteins.

32. The library according to claim 31 wherein the coding regions encode mammalian proteins.

33. The library according to claim 32 wherein the mammalian proteins are human proteins.

34. The library according to claim 33 wherein the human proteins are kinases, phosphatases, transcription factors, oncogenes, or tumor suppressors.

35. An expression library obtainable from the method of claim 1.

36. An expression vector pYES2/GS.

37. An expression vector pCDNA3.1/GS.

38. A method for producing a library of expressible coding regions comprising the steps of:
(a) amplifying a plurality of coding regions using PCR, wherein the 5′ primer comprises the sequence CACCATG and the 3′ primer causes the amplification product to end just prior to any stop codon,
(b) purifying the amplified coding regions using low melt agarose electrophoresis,
(c) inserting each of the purified coding regions into an expression vector using vaccinia DNA topoisomerase, wherein said expression vector comprises a promoter-enhancer sequence, a selection marker sequence, an origin of replication, an affinity purification sequence, and an epitope-tag sequence,
(d) transforming bacterial cells with the insert containing expression vector,
(e) growing the transformed cells and verifying the size and orientation of the inserted coding region,
(f) selecting expression vectors containing inserted coding regions in the correct orientation for expression of the gene product, and
(g) transforming cells for expression with said expression vectors.
